(19) United States
(12) Patent Application Publication
(10) Pub. No.: US 2026/0284179 A1
(43) Pub. Date: Sep. 24, 2026

(54) CORONAVIRUS VACCINES INDUCING BROAD IMMUNITY AGAINST VARIANTS

(71) Applicants: DIOSynVax Ltd, Hertford Hertfordshire (GB); Cambridge Enterprise Limited, Cambridge Cambridgeshire (GB)

(72) Inventors: Jonathan Luke HEENEY, Cambridge Cambridgeshire (GB); Sneha VISHWANATH, Cambridge Cambridgeshire (GB); Matteo FERRARI, Hertford Hertfordshire (GB); George CARNELL, Cambridge Cambridgeshire (GB)

(73) Assignees: DIOSynVax Ltd, Hertford Hertfordshire (GB); Cambridge Enterprise Limited, Cambridge Cambridgeshire (GB)

(21) Appl. No.: 19/471,911

(22) PCT Filed: Apr. 5, 2024
(86) PCT No.: PCT/GB2024/050925

§ 371 Date: Oct. 2, 2025

(30) Foreign Application Priority Data
Apr. 5, 2023 (GB) 2305091.7
Oct. 16, 2023 (GB) 2315811.6

(51) Int. Cl. A61K 39/215; A61K 39/00; A61P 37/04; C07K 14/005; C12N 15/86; G01N 33/569

(52) U.S. Cl. CPC A61K 39/215; A61P 37/04; C07K 14/005; C12N 15/86; G01N 33/56983; A61K 2039/53; C12N 2710/24143; C12N 2770/20022; C12N 2770/20034; G01N 2333/165; G01N 2469/10

(57) ABSTRACT

Designed coronavirus polypeptide sequences are described, and their use as vaccines against viruses of the coronavirus family. The designed sequences include designed coronavirus spike(S) proteins and fragments thereof, including CoV_S_T2_35 (SEQ ID 5 NO: 1), CoV_S_T2_36 (SEQ ID NO:2), and Omicron_vaccine (SEQ ID NO:3). Variants of CoV_S_T2_35 (deom) are also described, including CoV_S_T3_1 (SEQ ID NO:31) and CoV_S_T3_2 (SEQ ID NO:32), and fragments thereof. Nucleic acid molecules encoding the polypeptides, vectors, fusion proteins, pharmaceutical compositions, cells, and their use as vaccines against viruses of the coronavirus family are also described.

This invention relates to polynucleotides, polypeptides, vectors, cells, fusion proteins, pharmaceutical compositions, combined preparations, and their use as vaccines against viruses of the coronavirus family.

Coronaviruses (CoVs) cause a wide variety of animal and human disease. Notable human diseases caused by CoVs are zoonotic infections, such as severe acute respiratory syndrome (SARS) and Middle-East respiratory syndrome (MERS). Viruses within this family generally cause mild, self-limiting respiratory infections in immunocompetent humans, but can also cause severe, lethal disease characterised by onset of fever, extreme fatigue, breathing difficulties, anoxia, and pneumonia. CoVs transmit through close contact via respiratory droplets of infected subjects, with varying degrees of infectivity within each strain.

CoVs belong to the Coronaviridae family of viruses, all of which are enveloped. CoVs contain a single-stranded positive-sense RNA genome, with a length of between 25 and 31 kilobases (Siddell S. G. 1995, The Coronaviridae), the largest genome so far found in RNA viruses. The Coronaviridae family are subtyped into four genera: α, β, γ, and δ coronaviruses, based on phylogenetic clustering, with each genus subdivided again into clusters depending on the strain of the virus. For example, within the genus β-CoV (Group 2 CoV), four lineages (a, b, c, and d) are commonly recognized:
Lineage A (subgenus Embecovirus) includes HCoV-OC43 and HCoV-HKU1 (various species)
Lineage B (subgenus Sarbecovirus) includes SARSr-COV (which includes all its strains such as SARS-CoV, SARS-CoV-2, and Bat SL-CoV-WIV1)
Lineage C (subgenus Merbecovirus) includes Tylonycteris bat coronavirus HKU4 (BtCoV-HKU4), Pipistrellus bat coronavirus HKU5 (BtCoV-HKU5), and MERS-COV (various species)
Lineage D (subgenus Nobecovirus) includes Rousettus bat coronavirus HKU9 (BtCoV-HKU9)

A fifth subgenus is also officially recognised: Hibecovirus with one species of bat CoV.

CoV virions are spherical with characteristic club-shape spike projections emanating from the surface of the virion. The virions contain four main structural proteins: spike(S); membrane (M); envelope (E); and nucleocapsid (N) proteins, all of which are encoded by the viral genome. Some subsets of β-CoVs also comprise a fifth structural protein, hemagglutinin-esterase (HE), which enhances S protein-mediated cell entry and viral spread through the mucosa via its acetyl-esterase activity. Homo-trimers of the S glycoprotein make up the distinctive spike structure on the surface of the virus. These trimers are a class I fusion protein, mediating virus attachment to the host receptor by interaction of the S protein and its receptor. In most CoVs, S is cleaved by host cell protease into two separate polypeptides-S1 and S2. S1 contains the receptor-binding domain (RBD) of the S protein (the exact positioning of the RBD varies depending on the viral strain), while S2 forms the stem of the spike molecule.

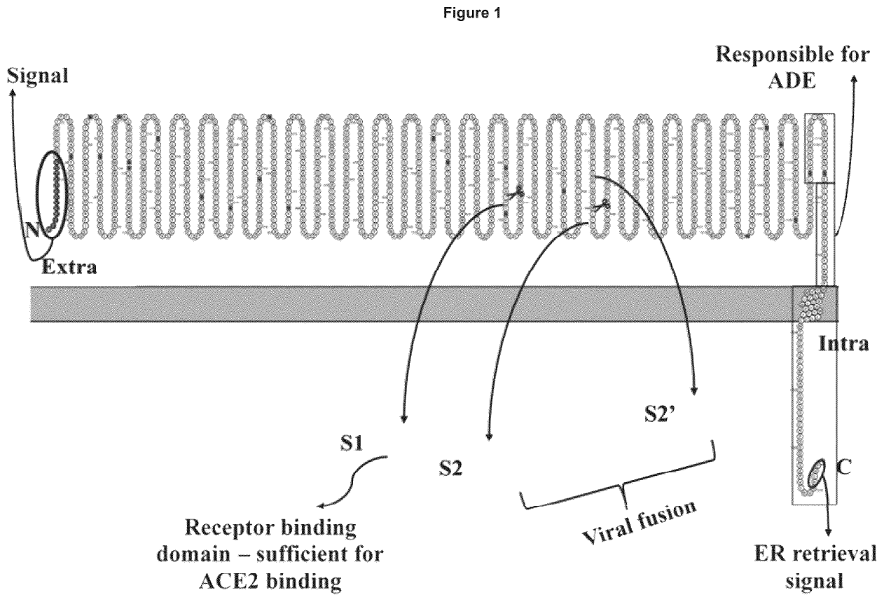

FIG. 1 shows SARS S-protein architecture. The N-terminal sequence is responsible for relaying extracellular signals intracellularly. Studies show that the N-terminal region of the S protein is much more diverse than the C-terminal region, which is highly conserved (Dong et al, Genomic and protein structure modelling analysis depicts the origin and infectivity of 2019-nCoV, a new coronavirus which caused a pneumonia outbreak in Wuhan, China. 2020). The figure shows the S domain, which comprises S1 and S2 domains, responsible for receptor binding and cell membrane fusion respectively.

The total length of SARS-CoV-2 S is 1273 amino acids and consists of a signal peptide (amino acids 1-13) located at the N-terminus, the S1 subunit (14-685 residues), and the S2 subunit (686-1273 residues); the last two regions are responsible for receptor binding and membrane fusion, respectively. SARS2 binds to human angiotensin converting enzyme 2 (ACE2) receptor for viral attachment and entry. In the S1 subunit, there is an N-terminal domain (14-305 residues) and a receptor-binding domain (RBD, 319-541 residues); the fusion peptide (FP) (788-806 residues), heptapeptide repeat sequence 1 (HR1) (912-984 residues), HR2 (1163-1213 residues), TM domain (1213-1237 residues), and cytoplasm domain (1237-1273 residues) comprise the S2 subunit. S protein trimers visually form a characteristic bulbous, crown-like halo surrounding the viral particle. Based on the structure of coronavirus S protein monomers, the S1 and S2 subunits form the bulbous head and stalk region. Structure of the SARS-CoV-2 trimeric S protein has been determined by cryo-electron microscopy/x-ray crystallography at the atomic level, revealing different conformations of the S RBD domain in opened and closed states and its corresponding functions.

RNA viruses generally have very high mutation rates compared to DNA viruses, because viral RNA polymerases lack the proofreading ability of DNA polymerases (although CoVs are an exception here as they possess a proofreading system: ExoN, nsp14). This is one reason why the virus can transmit from its natural host reservoir to other species, and from human to human, and is why it is difficult to make effective vaccines to prevent diseases caused by RNA viruses. In most cases, current vaccine candidates against RNA viruses are limited by the viral strain used as the vaccine insert, which is often chosen based on availability of a wild-type strain rather than by informed design. Technical challenges for developing vaccines for enveloped RNA viruses include: i) viral variation of wild-type field isolate glycoproteins (GPs) provide limited breadth of protection as vaccine antigens; ii) selection of vaccine antigens expressed by the vaccine inserts is highly empirical; immunogen selection is a slow, trial and error process; iii) in an evolving or unanticipated viral epidemic, developing new vaccine candidates is time-consuming and can delay vaccine deployment.

Before 2002, CoVs were only thought to cause mild respiratory problems, and were endemic in the human population, causing 15-30% of respiratory tract infections each year. Since their first discovery in the 1960's, the CoV family has expanded massively and has caused many outbreaks in both humans and animals. Towards the end of 2019, a novel CoV emerged; SARS-CoV-2, a group 2b β-CoV. The outbreak began in Wuhan, China in late 2019. By 30 January 2020, the WHO declared a global health emergency as the virus had spread to over 25 countries within a month of its emergence. The number of SARS2 infections increased exponentially across many countries around the world. Efforts to stop the spread of the virus were made, which curtailed the number of cases of infection and the number of deaths caused by the virus. However, multiple waves of the disease have occurred in many countries, resulting (by 14 Jul. 2022, according to the WHO) in global figures of more than 555 million confirmed cases of infection, and over 6 million confirmed deaths.

Since the first described human infection with SARS-CoV-2 in December of 2019, over 37 vaccines have been approved for use in humans, with many more in development (Craven, 2022, Regulatory Focus, News Articles, 2020, 3, COVID-19 Vaccine Tracker: https://www.raps.org/news-and-articles/news-articles/2020/3/covid-19-vaccine-tracker). The AstraZeneca/Oxford COVID-19 vaccine (AZD1222) uses an adenoviral vector. Two of the vaccines currently in use worldwide, BNT162b2 (BioNTech's vaccine manufactured by Pfizer) and mRNA-1273 (manufactured by Moderna), are based on lipid nanoparticle delivery of mRNA encoding a pre-fusion stabilized form of S protein derived from SARS-CoV-2 isolated early in the epidemic from Wuhan, China. Both of these vaccines demonstrated >94% efficacy at preventing coronavirus disease 2019 (COVID-19) in phase III clinical studies performed in late 2020 in multiple countries (Polack et al., C4591001 Clinical Trial Group (2020). Safety and Efficacy of the BNT162b2 mRNA Covid-19 Vaccine. N. Engl. J. Med. 383, 2603-2615; Baden et al., COVE Study Group (2021). Efficacy and Safety of the mRNA-1273 SARS-CoV-2 Vaccine. N. Engl. J. Med. 384, 403-416). However, the emergence of novel circulating variants has raised significant concerns about the effectiveness of the current vaccines, especially in countries where the epidemic is dominated by variant strains (Garcia-Beltran et al., 2021, Cell 184, 2372-2383: Multiple SARS-CoV-2 variants escape neutralization by vaccine-induced humoral immunity).

One of the earliest variants that emerged and rapidly became globally dominant was D614G. In the United Kingdom, a novel lineage termed B.1.1.7 (also known as VOC-202012/01 or 501Y.V1, or alpha) rapidly emerged. B.1.1.7 includes three amino acid deletions and seven missense mutations in spike, including D614G as well as N501Y in the ACE2 receptor-binding domain (RBD), and has been reported to be more infectious than variants with D614G mutation alone. There were also been reports of SARS-CoV-2 transmission between humans and minks in Denmark with a variant called mink cluster 5 or B.1.1.298, which includes a two-amino acid deletion and four missense mutations including Y453F in RBD. Another variant, which emerged in California, termed B.1.429, contains four missense mutations in the S protein, one of which is a single L452R RBD mutation. Novel variants arising from the B. 1.1.28 lineage first described in Brazil and Japan, termed P.2 (with 3 spike missense mutations) and P.1 (also termed Gamma variant, with 12 spike missense mutations), respectively, contain a E484K mutation. P.1 also contains K417T and N501Y mutations in the RBD. These strains spread rapidly, but are no longer detected, or detected at extremely low levels in the EU/EEA.

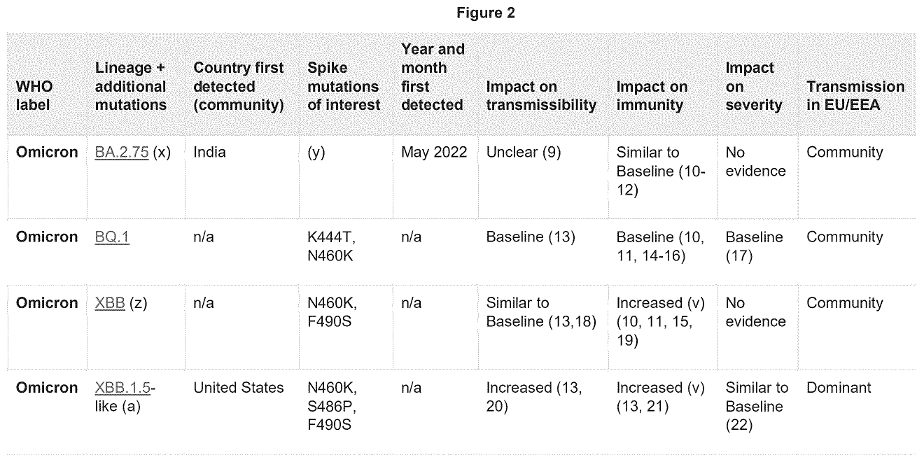

Of past concern was the emergence of multiple strains of the B.1.351 lineage (also known as Beta variant, or 501Y.V2), which were first reported in South Africa and spread globally. This lineage contains three RBD mutations, K417N, E484K, and N501Y, in addition to several mutations outside of RBD. B.1.617.2 (Delta variant) then emerged, comprising increased transmissibility. First detected in India in December 2020, the variant contains four mutations in the RBD: L452R, T478K, K417N, and E484K. More recently, the B.1.1.529 (BA. 1/Omicron) variant emerged, comprising 30 mutations in the S protein, 15 of which are in the RBD, which have shown to cause significant humoral immune evasion and high transmissibility. Since then, a number of sub-variants of Omicron have emerged, including BA.2. BA.3, BA.4, and BA.5. Some of these sub-variants also comprise sub-variants, including BA.2.12.1. BA.5 mutated into BQ. 1 and BQ.1.1, and BA.2 mutated into XBB1.5, which is able to bind more tightly to ACE-2 than its predecessors. FIG. 2 provides details of the variants of interest (VOI) in circulation globally as of 3 Apr. 2023 (https://www.ecdc.europa.eu/en/covid-19/variants-concern). At this point in time, there are no variants of concern in circulation, as many of the omicron sub-variants have been de-escalated by the European Centre for Disease Prevention and Control (ECDC).

BA.1, BA.2 and BA.3 share 12 RBD mutations, i.e., G339D, S373P, S375F, K417N, N440K, S477N, T478K, E484A, Q493R, Q498R, N501Y, and Y505H. BA.2 sub-lineage contains four additional mutations to BA.1, including S371F, T376A, D405N, and R408S, and lacks the S371L, G446S and G496S harboured by BA. 1. BA.3 has distinct mutations S371F, D405N, and G446S. Importantly, new Omicron variants are still continuously emerging. Mutants of BA.2 contain identical RBD sequences to BA.2 but with the addition of L452 and F486 substitutions, namely BA.2.12.1 (L452Q), BA.2.13 (L452M), BA.4 and BA.5 (L452R+F486V), and all display higher transmission advantage over BA.2. BA.4 and BA.5. Studies suggest that antibodies elicited by 3-dose vaccination (CoronaVac) are less effective at neutralising BA.4 and BA.5 than earlier Omicron strains. Furthermore, subjects with hybrid immunity, stemming from vaccination and previous infection with BA.1, produce antibodies that struggle to neutralise BA.4 and BA.5 (Cao, Y et al. Nature (2022). DOI: https://www.nature.com/articles/s41586-022-04980-y). Indeed, infection with BA. 1 induces a relatively narrow neutralising antibody response, which appears to leave subjects vulnerable to BA.4 and BA.5 infection. This is thought to be due to BA.4 and BA.5 variants' L452R and F486V S protein mutations. It is clear, therefore, that the population are at risk of infection by newly emerging SARS2 variants and sub-variants, wherein the SARS2 is unable to be effectively neutralised.

The emergence of novel variants that appear to escape immune responses has spurred vaccine manufacturers to develop boosters for these spike variants. However, the continued emergence of these VOCs during the on-going COVID-19 pandemic, and the constant threat of new zoonotic spill overs of coronaviruses from animals to humans, highlights the need for next generation vaccines with broader and more potent protection from ACE-2 binding Sarbecoviruses. In particular, there is a need to protect against more coronavirus variants and sub-variants than current vaccines, and to provide more potent protection against those variants and sub-variants. In particular, there is a need to provide improved vaccines that elicit more broadly neutralising immune responses to coronavirus omicron viruses, and emerging coronavirus omicron viruses. There is also a need to provide vaccines with stronger neutralising capacity against such viruses. Furthermore, there is a need to provide vaccines that successfully combat vaccine escape of new SARS-CoV-2 variants.

Thus there is a need to provide improved coronavirus vaccines that elicit broadly neutralising antibodies against SARS-CoV-2 variants, in particular against current and recent variants of concern. In particular there is a need to provide effective vaccines that induce a broadly neutralising immune response to protect against the Delta strain and several Omicron strains.

Furthermore, there is a need to provide vaccines that successfully combat vaccine escape of new SARS-CoV-2 variants.

### Designed SARS-CoV-2 Spike(s) Protein Sequences

The S protein of SARS-CoV-2 plays a key role in ACE-2 receptor recognition and fusion of the virus envelope with the host cell membrane. A comparison of 303,250 human SARS-CoV-2 spike protein sequences with the reference protein sequence Wuhan-Hu-1, showed ~96.5% of the spike protein sequence had undergone mutations (Guruprasad. Current Research in Structural Biology. Vol 4, 2022; 41-50) by February 2022, since the outbreak of the COVID-19 pandemic disease that was first reported in December 2019. A total of 1,269,629 mutations were detected corresponding to 1,229 distinct mutation sites in the spike proteins comprising 1,273 amino acid residues. Thereby, ~3.5% of the human SARS-CoV-2 spike protein sequence has remained invariant in the past two years. This figure will have decreased even more so since the document disclosing the comparison was published in early 2022. The S protein is the main antigen component of the structural proteins of SARS-CoV-2. Responsible for inducing the host immune response, nAbs targeting the S protein can induce protective immunity against viral infection.

As above, vaccines against SARS2 S protein exist, however these vaccines are unable to induce broadly neutralising immune responses against previous SARS2 variants and recently emerged SARS2 variants, including Omicron BA.4 and BA.5.

There is a need, therefore, to provide improved vaccines that elicit more broadly neutralising immune responses to SARS2 viruses, particularly against recently emerged and emerging SARS2 variants.

The Applicant has designed amino acid sequences that induce a broadly neutralising immune response against important SARS2 strains, including recently emerged sub-strains of Delta and Omicron. These designs are referred to herein as CoV_S_T2_35, and CoV_S_T2_36. Such polypeptides, and their encoding nucleic acid sequences, are particularly advantageous as they elicit broadly neutralising antibody responses to a panel of coronavirus viruses, including Wuhan, Alpha, Beta, Gamma, Delta, and Omicron BA. 1, BA.2, BA.2.12, and BA.4/5 pseudoviruses. The polypeptides and their encoding nucleic acid sequences also elicit broadly neutralising antibody responses against Omicron BA.2.75, BA.2.75.2, BA.2.3.20, BQ. 1.1, XBB, and XBB.1.5 pseudoviruses. In particular, CoV_S_T2_25 elicits a broadly neutralising antibody response against Omicron BA.2.75, BA.2.75.2, BA.2.3.20, BQ.1.1, XBB, and XBB.1.5 pseudoviruses. CoV_S_T2_26 elicits a broadly neutralising antibody response against Omicron BA.2.75 and BA.2.3.20 pseudoviruses.

The Applicant has also identified modifications to the amino acid sequences for better expression of the polypeptides of the invention. The Applicant has further identified amino acid residues responsible for increasing the stability of the polypeptides of the invention.

The Applicant has designed a further polypeptide sequence, Omicron_vaccine, based on the Omicron BA.1 consensus sequence, but including the amino acid modifications that the Applicant has identified as being important for vaccine polypeptide stability and expression.

S protein amino acid sequences according to the invention are described below.

### CoV_S_T2_35(deom) (SEQ ID NO: 1)

According to the invention there is provided an isolated polypeptide, which comprises the amino acid sequence of SEQ ID NO:1 (CoV_S_T2_35), or an amino acid which has at least 97%, 98%, or 99% amino acid identity over its entire length with the amino acid sequence of SEQ ID NO:1.

>COV_S_T2_35 (deom)
(SEQ ID NO: 1)
Amino acid sequence:
MFVFLVLLPLVSSQCVNLRTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLDVYYHK
NNKSWMESGVYSSANNCTFEYVSQPFLMDLEGKQGNFKNLREFVFKNID
GYFKIYSKHTPINLVRDLPQGFSALEPLVDLPIGINITRFQTLLALHRS
YLTPGDSSSGWTAGAAAYYVGYLQPRTFLLKYNENGTITDAVDCALDPL
SETKCTLKSFTVEKGIYQTSNFRVQPTESIVRFPNITNLCPFGEVENAT
RFASVYAWNRKRISNCVADYSVLYNSASFSTFKCYGVSPTKLNDLCFTN
VYADSFVIRGDEVRQIAPGQTGNIADYNYKLPDDFTGCVIAWNSNKLDS
KVSGNYNYLYRLFRKSNLKPFERDISTEIYQAGNKPCNGVAGFNCYFPL
RSYSFRPTYGVGHQPYRVVVLSFELLHAPATVCGPKKSTNLVKNKCVNF
NFNGLKGTGVLTESNKKFLPFQQFGRDIADTTDAVRDPQTLEILDITPC
SFGGVSVITPGTNTSNQVAVLYQGVNCTEVPVAIHADQLTPTWRVYSTG
SNVFQTRAGCLIGAEHVNNSYECDIPIGAGICASYQTHTNSRGSASSVA
SQSIIAYTMSLGAENSVAYSNNSIAIPTNFTISVTTEILPVSMTKTSVD
CTMYICGDSTECSNLLLQYGSFCTQLNRALTGIAVEQDKNTQEVFAQVK
QIYKTPPIKDFGGFNFSQILPDPSKPSKRSFIEDLLFNKVTLADAGFVK
QYGDCLGDIAARDLICAQKFNGLTVLPPLLTDEMIAQYTSALLAGTITS
GWTFGAGAALQIPFAMQMAYRFNGIGVTQNVLYENQKLIANQFNSAIGK
IQDSLSSTASALGKLQNVVNQNAQALNTLVKQLSSNFGAISSVLNDILS
RLDPPEAEVQIDRLITGRLQSLQTYVTQQLIRAAEIRASANLAATKMSE
CVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVV
IGIVNNTVYDPLQPELDSFKEELDKYFKNHTSPDVDLGDISGINASVVN
IQKEIDRLNEVAKNLNESLIDLQELGKYEQYIKWPWYIWLGFIAGLIAI
VMVTIMLCCMTSCCSCLKGCCSCGSCC

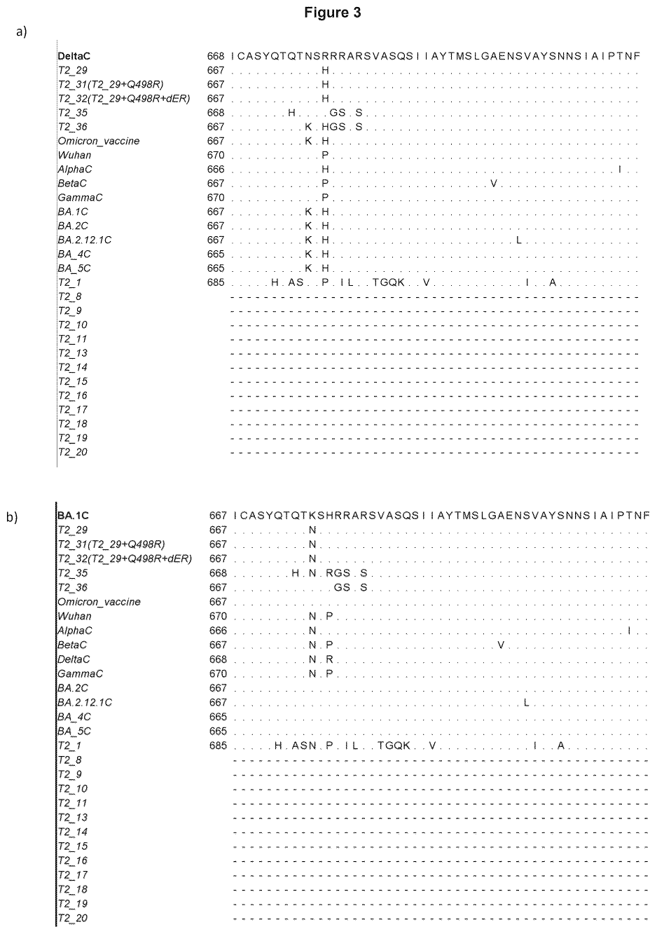

Alignment of the amino acid sequence of CoV_S_T2_35(deom) (SEQ ID NO:1) with coronavirus S protein reference sequences SEQ ID NOs: 4-13, and previously designed vaccine sequences SEQ ID NOs: 14-29, is shown in FIG. 3a. CoV_S_T2_35 is the most phylogenetically similar to the Delta variant of coronavirus, and so DeltaC is shown as the comparative reference sequence. The figure shows alignment of the aforementioned sequences at positions where CoV_S_T2_35 comprises novel amino acid residues, as discussed below. SEQ ID NOs: 5-13 are consensus sequences of wild-type (WT) strains of coronavirus. The amino acid differences of CoV_S_T2_35(deom) (SEQ ID NO:1) from the DeltaC S protein reference sequence (SEQ ID NO:8) are shown in Table 1 below:

TABLE 1

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | — | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 680 | R | G |
| 681 | R | S |
| 683 | R | S |
| 832 | | V |
| 984 | K | P |
| 985 | V | P |
| 1253-1271 | KFDEDDSEP | — (deletion) |
| | VLKGVKLHYT | |
| Total No of | — | 39/1271 |
| differences from | | |
| reference | | |
| Percentage | — | 96.93% |
| identity with the | | |
| reference | | |

The amino acid residues involved in stabilising the polypeptide design are in bold format. Of these residues, the novel amino acid residues are shown in bold and underline format. The deletion of amino acids corresponding to residues 1253-1271 of SEQ ID NO:8 are shown as “deletion”. The deletion of these residues promotes expression of the design. The residue positions shown in the table correspond to the reside positions of both DeltaC and CoV_S_T2_35 sequences.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least one of the amino acid residues of SEQ ID NO:1, at positions corresponding to the amino acid residue positions of SEQ ID NO:8, as shown in Table 1.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least five of the amino acid residues of SEQ ID NO: 1, at positions corresponding to the amino acid residue positions of SEQ ID NO:8, as shown in Table 1.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least ten of the amino acid residues of SEQ ID NO:1, at positions corresponding to the amino acid residue positions of SEQ ID NO:8, as shown in Table 1.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least fifteen of the amino acid residues of SEQ ID NO:1, at positions corresponding to the amino acid residue positions of SEQ ID NO:4, as shown in Table 1.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least twenty of the amino acid residues of SEQ ID NO:1, at positions corresponding to the amino acid residue positions of SEQ ID NO:8, as shown in Table 1.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises the amino acid residues of SEQ ID NO:1, at positions corresponding to the amino acid residue positions of SEQ ID NO:8, as shown in Table 1.

The amino acid differences of CoV_S_T2_35(deom) (SEQ ID NO: 1) from the DeltaC S protein reference sequence (SEQ ID NO:8) which are novel, are shown in Table 3 below. The residue positions shown in the table below correspond to the residue positions for both DeltaC and CoV_S_T2_35.

TABLE 3

| | DeltaC (SEQ | |
|---|---|---|
| DeltaC (SEQ ID | ID NO: 8) | CoV_S_T2_35 |
| NO: 8) residue | reference | (deom) SEQ ID |
| position | residue | NO: 1) |
| 680 | R | G |
| 681 | R | S |
| 683 | R | S |

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises at least one of the amino acid residues of SEQ ID NO:1, at positions corresponding to the amino acid residue positions of SEQ ID NO:8, as shown in Table 3.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises the amino acid residues of SEQ ID NO:1, at positions corresponding to the amino acid residue positions of SEQ ID NO:8, as shown in Table 3.

The Applicant has appreciated that polypeptides of the invention which comprise the above residue changes have better stability than polypeptides comprising the wild-type residues at these positions.

The amino acid differences of CoV_S_T2_35(deom) (SEQ ID NO: 1) from the DeltaC S protein reference sequence (SEQ ID NO:8), which are present in at least one reference sequence or previously designed (DIOS) sequence, are shown in table 2 below. Generally, these amino acid differences are found in wild-type coronavirus sequences, and their incorporation into the vaccine designs ensures that variants comprising the amino acid changes are captured by the vaccine design.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises the amino acid residues of SEQ ID NO:1, at positions corresponding to the amino acid residue positions of SEQ ID NO:8, as shown in Table 2.

TABLE 2

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 832 | I | V |
| 984 | K | P |
| 985 | V | P |

The Applicant has found that such polypeptides elicit broadly neutralising immune responses against a panel of SARS-CoV-2 pseudoviruses, including Wuhan, Alpha, Beta, Gamma, Delta, and one or more of Omicron sub-strains BA. 1, BA.2, BA.2.12.1, and BA.4/5. The polypeptides also elicit broadly neutralising antibody responses against Omicron BA.2.75, BA.2.75.2, BA.2.3.20, BA.2.12, BQ.1.1, XBB, and XBB.1.5 pseudoviruses. In particular, CoV_S_T2_25 elicits a broadly neutralising antibody response against Omicron BA.2.75, BA.2.75.2, BA.2.3.20, BQ.1.1, XBB, and XBB.1.5 pseudoviruses. CoV_S_T2_26 elicits a broadly neutralising antibody response against Omicron BA.2.75 and BA.2.3.20 pseudoviruses. BA.4 and BA.5 were recently (as of April 2023) the dominant SARS-CoV-2 strains circulating in many countries around the world.

Optionally, a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises the following amino acid residues at positions corresponding to residues 984, and 985, of SEQ ID NO: 8:
984; P; and
985; P.

Advantageously, a proline residue at positions 984 and 985 increases the stability of the vaccine design.

Optionally, a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, comprises the following amino acid residues at positions corresponding to residues 680, 681, 683, 984, and 985, of SEQ ID NO:8:
680; G;
681; S;
683; S;
984; P; and
985; P.

The Applicant has appreciated that polypeptides of the invention which comprise the above residue changes have better stability than polypeptides comprising the wild-type residues at these positions.

Optionally a polypeptide of the invention, which comprises the amino acid sequence of SEQ ID NO: 1 (CoV_S_T2_35), or an amino acid which has at least 97%, 98%, or 99% amino acid identity over its entire length with the amino acid sequence of SEQ ID NO:1, does not include residues KFDEDDSEPVLKGVKLHYT at positions corresponding to amino acid residue positions 1253-1271 of SEQ ID NO:8.

The Applicant has appreciated that deletion of 19 amino acid residues from the C-terminus of the SARS-CoV-2 S protein at positions corresponding to residue positions 1253-1271 of SEQ ID NO: 8 (endoplasmic reticulum (ER) signal sequence) improves the expression of the designed amino acid sequence.

According to the invention there is provided an isolated polypeptide which comprises CoV_S_T2_35(deom) amino acid sequence (SEQ ID NO:1).

According to the invention there is also provided an isolated polypeptide which comprises a coronavirus S protein with at least one, or all of the amino acid residues, at positions corresponding to the amino acid residue positions of SEQ ID NO:8, as shown in Table 3 below:

TABLE 3

| DeltaC (SEQ | DeltaC (SEQ | |
|---|---|---|
| ID NO: 8) | ID NO: 8) | CoV_S_T2_35 |
| residue | reference | (deom) SEQ ID |
| position | residue | NO: 1) |
| 680 | R | G |
| 681 | R | S |
| 683 | R | S |

Optionally there is provided an isolated polypeptide which comprises a coronavirus S protein which comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:8, and which comprises at least one, or all of the amino acid residues, at positions corresponding to the amino acid residue positions of SEQ ID NO: 8, as shown in Table 3.

Optionally an isolated polypeptide which comprises a coronavirus S protein, according to the invention, comprises the following amino acid residues at positions corresponding to residues 984, and 985, of SEQ ID NO:8:
984; P; and
985; P.

Optionally an isolated polypeptide which comprises a coronavirus S protein, according to the invention, comprises the following amino acid residues at positions corresponding to residues 680, 681, 683, 984, and 985, of SEQ ID NO:8:
680; G;
681; S;
683; S;
984; P; and
985; P.

Optionally there is provided an isolated polypeptide which comprises a coronavirus S protein, comprising the amino acid residues of SEQ ID NO: 1, at positions corresponding to the amino acid residue positions of SEQ ID NO:8, as shown in Table 2 below:

TABLE 2

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 832 | I | V |
| 984 | K | P |
| 985 | V | P |

Optionally there is provided an isolated polypeptide which comprises a coronavirus S protein, comprising the amino acid residues of SEQ ID NO: 1, at positions corresponding to the amino acid residue positions of SEQ ID NO:8, as shown in Table 1 below:

TABLE 1

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 680 | R | G |
| 681 | R | S |
| 683 | R | S |
| 832 | I | V |
| 984 | K | P |
| 985 | V | P |

Optionally a coronavirus S protein according to the invention does not include residues KFDEDDSEPVLKGVKLHYT at positions corresponding to amino acid residue positions 1253-1271 of SEQ ID NO:8.

### CoV_S_T2_35 Scaffold Sequence

We have also designed a scaffold S protein polypeptide sequence (SEQ ID NO:30), based on CoV_S_T2_35(deom) (SEQ ID NO:1), which includes constant regions of sequence, and variable amino acid residues. The variable amino acid residues can be changed to provide different antigens which induce a neutralising immune response against new SARS-CoV-2 variants as they arise (and/or against future SARS-CoV-2 variants).

SEQ ID NO:30 below shows a scaffold S protein sequence in which the amino acid sequence of the constant regions of the scaffold is provided, with each variable amino acid residue represented with an X (shown underlined in the sequence below):

| | |
|---|---|
| MFVFLVLLPL VSSQCVNLRT RTQLPPAYTN SFTRGVYYPD KVFRSSVLHS TQDLFLPFFS | 60 |
| NVTWFHAIHV SGTNGTKRED NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNFRVQP TESIVRFPNI TNLCPFXEVF NATXFASVYA WNRKRISNCV | 360 |
| ADYSVXYNSA XFXXFKCYGV SPTKLNDLCF TNVYADSFVI RGXEVXQIAP GQTGNIADYN | 420 |
| YKLPDDFTGC VIAWNSNKLD SKXXGNYNYX YRLERKSXLK PFERDISTEI YQAGNKPCNG | 480 |
| VAGXNCYXPL XSYXFRPTYG VGHQPYRVVV LSFELLHAPA TVCGPKKSTN LVKNKCVNEN | 540 |
| FNGLKGTGVL TESNKKELPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHINSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVF | 780 |
| AQVKQIYKTP PIKDFGGFNF SQILPDPSKP SKRSFIEDLL ENKVTLADAG FVKQYGDCLG | 840 |
| DIAARDLICA QKFNGLTVLP PLLTDEMIAQ YTSALLAGTI TSGWTFGAGA ALQIPFAMQM | 900 |
| AYRENGIGVT QNVLYENQKL IANQFNSAIG KIQDSLSSTA SALGKLQNVV NQNAQALNTL | 960 |
| VKQLSSNEGA ISSVLNDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |

In this sequence X may be any amino acid residue.

CoV_S_T2_35(deom) (SEQ ID NO:1) is an example of a polypeptide sequence covered by the scaffold sequence. Other examples of polypeptide sequences covered by the scaffold sequence are shown below:

| COV_S_T3_1(Deom_v2) (SEQ ID NO: 31): | |
|---|---|
| MFVFLVLLPL VSSQCVNLRT RTQLPPAYTN SFTRGVYYPD KVFRSSVLHS TQDLFLPFFS | 60 |
| NVTWFHAIHV SGTNGTKRED NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNFRVQP TESIVRFPNI TNLCPFDEVE NATRFASVYA WNRKRISNCV | 360 |
| ADYSVLYNFA PFFAFKCYGV SPTKLNDLCF TNVYADSFVI RGNEVSQIAP GQTGNIADYN | 420 |
| YKLPDDFTGC VIAWNSNKLD SKVGGNYNYR YRLERKSKLK PFERDISTEI YQAGNKPCNG | 480 |
| VAGPNCYFPL QSYGFRPTYG VGHQPYRVVV LSFELLHAPA TVCGPKKSTN LVKNKCVNEN | 540 |
| FNGLKGTGVL TESNKKELPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHTNSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVF | 780 |
| AQVKQIYKTP PIKDFGGENF SQILPDPSKP SKRSFIEDLL FNKVTLADAG FVKQYGDCLG | 840 |
| DIAARDLICA QKFNGLTVLP PLLTDEMIAQ YTSALLAGTI TSGWTFGAGA ALQIPFAMQM | 900 |
| AYRFNGIGVT QNVLYENQKL IANQFNSAIG KIQDSLSSTA SALGKLQNVV NQNAQALNTL | 960 |
| VKQLSSNEGA ISSVLNDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |
| COV_S_T3_2(Deom_v3) (SEQ ID NO: 32): | |
| MFVFLVLLPL VSSQCVNLRT RTQLPPAYTN SFTRGVYYPD KVFRSSVLHS TQDLFLPFFS | 60 |
| NVTWFHAIHV SGTNGTKRED NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNERVQP TESIVRFPNI TNLCPFHEVE NATTFASVYA WNRKRISNCV | 360 |
| ADYSVIYNFA PFFAFKCYGV SPTKLNDLCF TNVYADSFVI RGNEVSQIAP GQTGNIADYN | 420 |
| YKLPDDFTGC VIAWNSNKLD SKPSGNYNYL YRLERKSKLK PFERDISTEI YQAGNKPCNG | 480 |
| VAGPNCYSPL QSYGFRPTYG VGHQPYRVVV LSFELLHAPA TVCGPKKSTN LVKNKCVNEN | 540 |
| FNGLKGTGVL TESNKKFLPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHTNSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVF | 780 |
| AQVKQIYKTP PIKDFGGENF SQILPDPSKP SKRSFIEDLL FNKVTLADAG FVKQYGDCLG | 840 |
| DIAARDLICA QKFNGLTVLP PLLTDEMIAQ YTSALLAGTI TSGWTFGAGA ALQIPFAMQM | 900 |
| AYRENGIGVT QNVLYENQKL IANQFNSAIG KIQDSLSSTA SALGKLQNVV NQNAQALNTL | 960 |
| VKQLSSNFGA ISSVINDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |

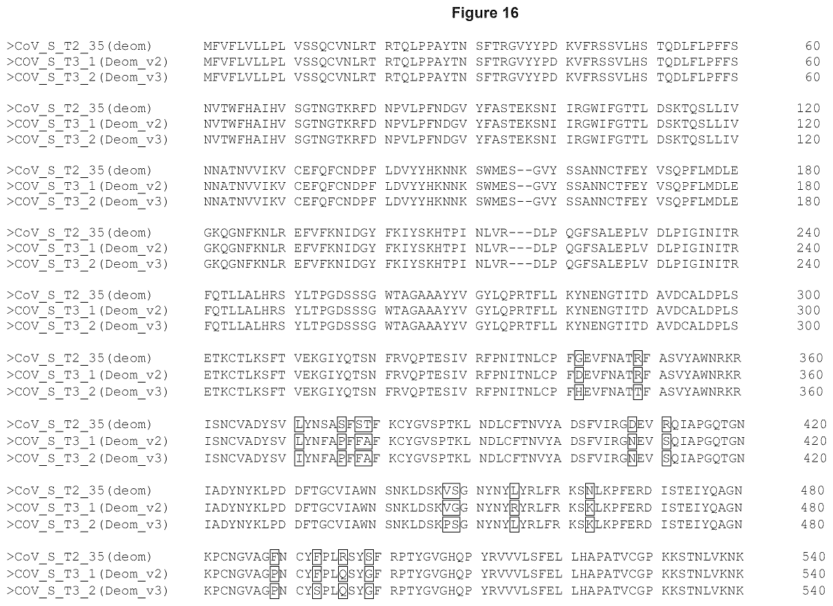

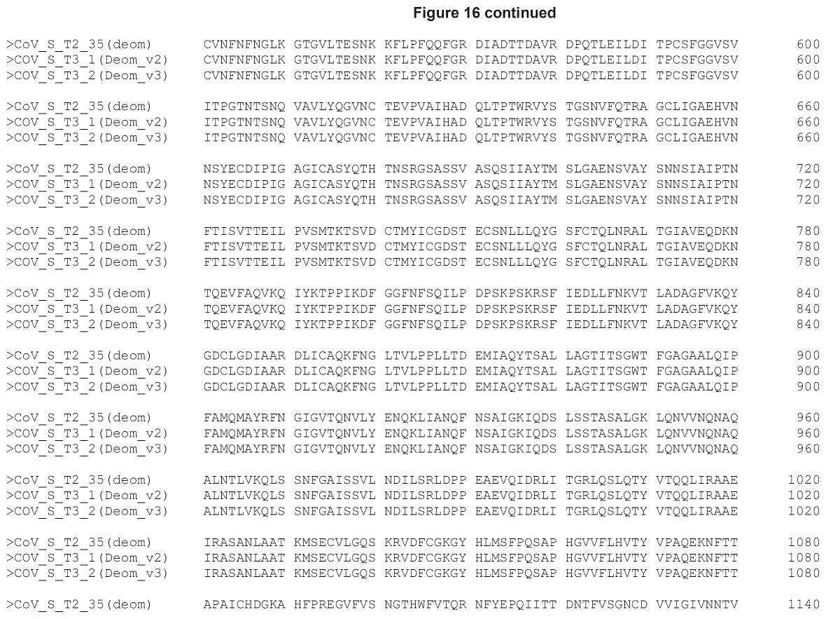

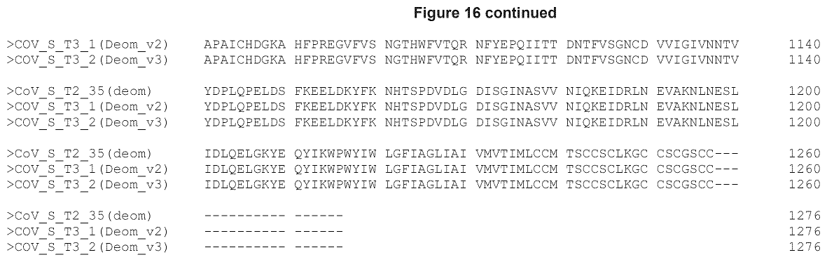

FIG. 16 shows an amino acid sequence alignment of CoV_S_T2_35(deom) (SEQ ID NO:1) with CoV_S_T3_1(Deom_v2) (SEQ ID NO:31), and CoV_S_T3_2(Deom_v3) (SEQ ID NO: 32). Differences between the sequences are shown as the boxed residues.

The residues at the variable positions in the CoV_S_T2_35(deom) (SEQ ID NO:1), CoV_S_T3_1(Deom_v2) (SEQ ID NO:31), and CoV_S_T3_2(Deom_v3) (SEQ ID NO:32) amino acid sequences are listed in the table below:

| | Residue at | | |
|---|---|---|---|
| | corresponding | | |
| Variable amino acid | position of | | |
| residue position of | CoV_S_T2_35(deom) | COV_S_T3_1(Deom_v2) | COV_S_T3_2(Deom_v3) |
| SEQ ID NO: 30 | (SEQ ID NO: 1) | (SEQ ID NO: 31) | (SEQ ID NO: 32) |
| 337 | G | D | H |
| 344 | R | R | T |
| 366 | L | L | I |
| 371 | S | P | P |
| 373 | S | F | F |
| 374 | T | A | A |
| 403 | D | N | N |
| 406 | R | S | S |
| 443 | V | V | P |
| 444 | S | G | S |
| 450 | L | R | L |
| 458 | N | K | K |
| 484 | F | P | P |
| 488 | F | F | S |
| 491 | R | Q | Q |
| 494 | S | G | G |

According to the invention there is provided an isolated polypeptide which comprises an amino acid sequence of SEQ ID NO:30 (CoV_S_T2_35 Scaffold Sequence), wherein X at amino acid residue positions 337, 344, 366, 371, 373, 374, 403, 406, 443, 444, 450, 458, 484, 488, 491, and 494, is any amino acid residue.

The RBD portion of SEQ ID NO:30 is from residues 317-530 (shown below as SEQ ID NO:33):

| | |
|---|---|
| RVQPTESIVR FPNITNLCPF XEVENATXFA SVYAWNRKRI SNCVADYSVX YNSAXFXXFK | 60 |
| CYGVSPTKLN DLCFTNVYAD SFVIRGXEVX QIAPGQTGNI ADYNYKLPDD FTGCVIAWNS | 120 |
| NKLDSKXXGN YNYXYRLFRK SXLKPFERDI STEIYQAGNK PCNGVAGXNC YXPLXSYXFR | 180 |
| PTYGVGHQPY RVVVLSFELL HAPATVCGPK KSTN | 214 |

According to the invention there is also provided an isolated polypeptide which comprises an amino acid sequence of SEQ ID NO:33 (RBD portion of CoV_S_T2_35 Scaffold Sequence (SEQ ID NO:30)), wherein X at amino acid residue positions corresponding to amino acid residue positions 337, 344, 366, 371, 373, 374, 403, 406, 443, 444, 450, 458, 484, 488, 491, and 494, of SEQ ID NO:30, is any amino acid residue.

Optionally an isolated polypeptide of the invention which comprises an amino acid sequence of SEQ ID NO:30 or 33 comprises amino acid residue G, D, or H at the amino acid residue position corresponding to position 337 of SEQ ID NO:30.

Optionally an isolated polypeptide of the invention which comprises an amino acid sequence of SEQ ID NO:30 or 33 comprises amino acid residue R or T at the amino acid residue position corresponding to position 344 of SEQ ID NO:30.

Optionally an isolated polypeptide of the invention which comprises an amino acid sequence of SEQ ID NO:30 or 33 comprises amino acid residue L or I at the amino acid residue position corresponding to position 366 of SEQ ID NO:30.

Optionally an isolated polypeptide of the invention which comprises an amino acid sequence of SEQ ID NO:30 or 33 comprises amino acid residue comprises amino acid residue S or P at the amino acid residue position corresponding to position 371 of SEQ ID NO:30.

Optionally an isolated polypeptide of the invention which comprises an amino acid sequence of SEQ ID NO:30 or 33 comprises amino acid residue S or F at the amino acid residue position corresponding to position 373 of SEQ ID NO:30.

Optionally an isolated polypeptide of the invention which comprises an amino acid sequence of SEQ ID NO:30 or 33 comprises amino acid residue T or A at the amino acid residue position corresponding to position 374 of SEQ ID NO:30.

Optionally an isolated polypeptide of the invention which comprises an amino acid sequence of SEQ ID NO:30 or 33 comprises amino acid residue D or N at the amino acid residue position corresponding to position 403 of SEQ ID NO:30.

Optionally an isolated polypeptide of the invention which comprises an amino acid sequence of SEQ ID NO:30 or 33 comprises amino acid residue R or S at the amino acid residue position corresponding to position 406 of SEQ ID NO:30.

Optionally an isolated polypeptide of the invention which comprises an amino acid sequence of SEQ ID NO:30 or 33 comprises amino acid residue V or P at the amino acid residue position corresponding to position 443 of SEQ ID NO:30.

Optionally an isolated polypeptide of the invention which comprises an amino acid sequence of SEQ ID NO:30 or 33 comprises amino acid residue S or G at the amino acid residue position corresponding to position 444 of SEQ ID NO:30.

Optionally an isolated polypeptide of the invention which comprises an amino acid sequence of SEQ ID NO:30 or 33 comprises amino acid residue L or R at the amino acid residue position corresponding to position 450 of SEQ ID NO:30.

Optionally an isolated polypeptide of the invention which comprises an amino acid sequence of SEQ ID NO:30 or 33 comprises amino acid residue N or K at the amino acid residue position corresponding to position 458 of SEQ ID NO:30.

Optionally an isolated polypeptide of the invention which comprises an amino acid sequence of SEQ ID NO:30 or 33 comprises amino acid residue F or P at the amino acid residue position corresponding to position 484 of SEQ ID NO:30.

Optionally an isolated polypeptide of the invention which comprises an amino acid sequence of SEQ ID NO:30 or 33 comprises amino acid residue F or S at the amino acid residue position corresponding to position 488 of SEQ ID NO:30.

Optionally an isolated polypeptide of the invention which comprises an amino acid sequence of SEQ ID NO:30 or 33 comprises amino acid residue R or Q at the amino acid residue position corresponding to position 491 of SEQ ID NO:30.

Optionally an isolated polypeptide of the invention which comprises an amino acid sequence of SEQ ID NO:30 or 33 comprises amino acid residue S or G at the amino acid residue position corresponding to position 494 of SEQ ID NO:30.

Optionally an isolated polypeptide of the invention which comprises an amino acid sequence of SEQ ID NO:30 comprises an amino acid sequence of SEQ ID NO:31.

Optionally an isolated polypeptide of the invention which comprises an amino acid sequence of SEQ ID NO:30 comprises an amino acid sequence of SEQ ID NO:32.

The RBD portion of SEQ ID NO:1 (CoV_S_T2_35(deom)) is shown below as SEQ ID NO:34:

| | |
|---|---|
| RVQPTESIVR FPNITNLCPF GEVENATRFA SVYAWNRKR ISNCVADYSVL YNSASFSTEK | 60 |
| CYGVSPTKLN DLCFTNVYAD SFVIRGDEVR QIAPGQTGN IADYNYKLPDD FTGCVIAWNS | 120 |
| NKLDSKVSGN YNYLYRLFRK SNLKPFERDISTEIYQAGNK PCNGVAGENC YFPLRSYSFR | 180 |
| PTYGVGHQPY RVVVLSFELL HAPATVCGPK KSTN | 214 |

The RBD portion of SEQ ID NO:31 (CoV_S_T3_1(Deom_v2) (SEQ ID NO:31)) is shown below as SEQ ID NO:35:

| | |
|---|---|
| RVQPTESIVR FPNITNLCPF DEVENATRFA SVYAWNRKRI SNCVADYSVL YNFAPFFAFK | 60 |
| CYGVSPTKLN DLCFTNVYAD SFVIRGNEVS QIAPGQTGNI ADYNYKLPDD FTGCVIAWNS | 120 |
| NKLDSKVGGN YNYRYRLFRK SKLKPFERDI STEIYQAGNK PCNGVAGPNC YFPLQSYGFR | 180 |
| PTYGVGHQPY RVVVLSFELL HAPATVCGPK KSTN | 214 |

The RBD portion of SEQ ID NO:32 (CoV_S_T3_2(Deom_v3) (SEQ ID NO:32)) is shown below as SEQ ID NO:36:

| | |
|---|---|
| RVQPTESIVR FPNITNLCPF HEVENATTFA SVYAWNRKR ISNCVADYSVI YNFAPFFAFK | 60 |
| CYGVSPTKLN DLCFTNVYAD SFVIRGNEVS QIAPGQTGN IADYNYKLPDD FTGCVIAWNS | 120 |
| NKLDSKPSGN YNYLYRLFRK SKLKPFERDISTEIYQAGNK PCNGVAGPNC YSPLQSYGER | 180 |
| PTYGVGHQPY RVVVLSFELL HAPATVCGPK KSTN | 214 |

Optionally an isolated polypeptide of the invention which comprises an amino acid sequence of SEQ ID NO:33 comprises an amino acid sequence of SEQ ID NO:34.

Optionally an isolated polypeptide of the invention which comprises an amino acid sequence of SEQ ID NO:33 comprises an amino acid sequence of SEQ ID NO:35.

Optionally an isolated polypeptide of the invention which comprises an amino acid sequence of SEQ ID NO:33 comprises an amino acid sequence of SEQ ID NO:36.

The RBD portion of SEQ ID NO: 1, 30, 31, or 32 may be provided without additional SARS-CoV-2 S protein sequence, or as part of a longer polypeptide with SARS-CoV-2 S protein sequence, for example as part of a full-length SARS-CoV-2 S protein. The S protein sequence may be sequence from an S protein disclosed herein, or another S protein sequence. For example, an S protein comprising an RBD of SEQ ID NO:33, 34, 35, or 36 may have an amino acid sequence that has at least 70%, 75%, 80%, 85%, 90%, 91%, 92%, 93%, 94%, 95%, 96%, 97%, 98%, or 99% amino acid identity over its entire length with the amino acid sequence of DeltaC S protein (SEQ ID NO:8). Alternatively, an S protein comprising an RBD of SEQ ID NO: 33, 34, 35, or 36 may have an amino acid sequence that has at least 70%, 75%, 80%, 85%, 90%, 91%, 92%, 93%, 94%, 95%, 96%, 97%, 98%, or 99% amino acid identity over its entire length with the amino acid sequence of BA. 1C S protein (SEQ ID NO:9).

### Cov_S_T2_36 (Omide) (Seq Id No:2)

According to the invention there is provided isolated polypeptide, which comprises the amino acid sequence of SEQ ID NO:2 (CoV_S_T2_36), or an amino acid which has at least 97%, 98%, or 99% amino acid identity over its entire length with the amino acid sequence of SEQ ID NO: 2.

>CoV_S_T2_36 (omide)
(SEQ ID NO: 2)
Amino acid sequence:
MFVFLVLLPLVSSQCVNLTTRTQLPPAYTNSFTRGVYYPDKVFRSSVLH
STQDLFLPFFSNVTWFHVISGTNGTKRFDNPVLPFNDGVYFASIEKSNI
IRGWIFGTTLDSKTQSLLIVNNATNVVIKVCEFQFCNDPFLDHKNNKSW
MESEFRVYSSANNCTFEYVSQPFLMDLEGKQGNFKNLREFVFKNIDGYF
KIYSKHTPIIVREPEDLPQGFSALEPLVDLPIGINITRFQTLLALHRSY
LTPGDSSSGWTAGAAAYYVGYLQPRTFLLKYNENGTITDAVDCALDPLS
ETKCTLKSFTVEKGIYQTSNFRVQPTESIVRFPNITNLCPFGEVFNATR
FASVYAWNRKRISNCVADYSVLYNSASFSTFKCYGVSPTKLNDLCFTNV
YADSFVIRGDEVRQIAPGQTGNIADYNYKLPDDFTGCVIAWNSNNLDSK
VGGNYNYRYRLFRKSNLKPFERDISTEIYQAGSKPCNGVEGFNCYFPLQ
SYGFQPTYGVGYQPYRVVVLSFELLHAPATVCGPKKSTNLVKNKCVNFN
FNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGS
NVFQTRAGCLIGAEYVNNSYECDIPIGAGICASYQTQTKSHGSASSVAS
QSIIAYTMSLGAENSVAYSNNSIAIPTNFTISVTTEILPVSMTKTSVDC
TMYICGDSTECSNLLLQYGSFCTQLKRALTGIAVEQDKNTQEVFAQVKQ
IYKTPPIKYFGGFNFSQILPDPSKPSKRSFIEDLLFNKVTLADAGFIKQ
YGDCLGDIAARDLICAQKFKGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNHNAQALNTLVKQLSSKFGAISSVLNDIFSR
LDPPEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCC

Alignment of the amino acid sequence of CoV_S_T2_36 (omide) (SEQ ID NO:9) with coronavirus S protein reference sequences SEQ ID NOs: 4-13, and previously designed vaccine sequences SEQ ID NOs: 14-29, is shown in FIG. 3b. CoV_S_T2_36 is the most phylogenetically similar to the Omicron BA. 1 variant of coronavirus, and so BA. 1 is shown as the comparative reference sequence. The figure shows alignment of the aforementioned sequences at positions where CoV_S_T2_36 comprises novel amino acid residues, as discusses below. SEQ ID NOs: 5-13 are consensus sequences of wild-type (WT) strains of coronavirus. The amino acid differences of CoV_S_T2_36 (omide) (SEQ ID NO:2) from the Omicron BA. 1C S protein reference sequence (SEQ ID NO:9) are shown in Table 4 below:

TABLE 4

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ |
| position | residue | ID NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 679 | R | G |
| 680 | R | S |
| 682 | R | S |
| 983 | K | P |
| 984 | V | P |
| 1252-1270 | KFDEDD | — (deletion) |
| | SEPVLK | |
| | GVKLHYT | |
| Total No of | — | 38/1271 |
| differences | | |
| from | | |
| reference | | |
| Percentage | — | 97.01% |
| identity with | | |
| the reference | | |

The amino acid residues involved in stabilising the polypeptide design are in bold format. Of these residues, the novel amino acid residues of CoV_S_T2_36 are shown in bold and underline format. The deletion of amino acids corresponding to residues 1252-1270 of SEQ ID NO: 8 are shown as “deletion”. The deletion of these residues promotes expression of the designed polypeptide sequence. The residue positions shown in the table correspond to the reside positions of both BA. 1C and CoV_S_T2_36 sequences.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least one, or all of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least five of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least ten of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least fifteen of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least twenty of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least twenty-five of the amino acid residues of SEQ ID NO:2, at positions corresponding to the amino acid residue positions of SEQ ID NO:9, as shown in Table 4.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises the amino acid residues of SEQ ID NO:2, at positions corresponding to the amino acid residue positions of SEQ ID NO:9, as shown in Table 4.

The amino acid differences of CoV_S_T2_36 (omide) (SEQ ID NO:2) from the Omicron BA. 1C S protein reference sequence (SEQ ID NO:9), which are novel in CoV-S-T2_36 (omide) vaccine design, are shown in Table 6 below. As explained above, these residue changes stabilise the S protein designed sequence.

TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) |
| position | residue | SEQ ID NO: 2) |
| 679 | R | G |
| 680 | R | S |
| 682 | R | S |

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises at least one of the amino acid residues of SEQ ID NO:2, at positions corresponding to the amino acid residue positions of SEQ ID NO:9, as shown in Table 6.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises the amino acid residues of SEQ ID NO:2, at positions corresponding to the amino acid residue positions of SEQ ID NO:9, as shown in Table 6.

The Applicant has appreciated that polypeptides of the invention which comprise the above residue changes have better stability than polypeptides comprising the wild-type residues at these positions.

The amino acid differences of CoV_S_T2_36 (omide) (SEQ ID NO:2) from the Omicron BA. 1C S protein reference sequence (SEQ ID NO:9), wherein the amino acid difference is present in at least one reference sequence or previously designed (DIOS) sequence, are shown in Table 5 below. Generally, these amino acid differences are found in wild-type coronavirus sequences, and their incorporation into the vaccine designs ensures that variants comprising the amino acid changes are captured by the vaccine design.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises the amino acid residues of SEQ ID NO:2, at positions corresponding to the amino acid residue positions of SEQ ID NO:9, as shown in Table 5.

TABLE 5

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) |
| position | residue | SEQ ID NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 983 | K | P |
| 984 | V | P |

The Applicant has found that such polypeptides elicit broadly neutralising immune responses against a diverse panel of SARS-CoV-2 pseudoviruses, including Wuhan, Alpha, Beta, Gamma, Delta, and Omicron sub-strains BA. 1, BA.2, BA.2.12.1, and BA.4/5. The polypeptides also elicit broadly neutralising antibody responses against Omicron BA.2.75, BA.2.75.2, BA.2.3.20, BQ.1.1, XBB, and XBB.1.5 pseudoviruses. In particular, CoV_S_T2_25 elicits a broadly neutralising antibody response against Omicron BA.2.75, BA.2.75.2, BA.2.3.20, BQ.1.1, XBB, and XBB.1.5 pseudoviruses. CoV_S_T2_26 elicits a broadly neutralising antibody response against Omicron BA.2.75 and BA.2.3.20 pseudoviruses. As above, BA.4 and BA.5 are the dominant (as of April 2023) SARS-CoV-2 strains currently circulating in many countries around the world.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises the following amino acid residues at positions corresponding to residues 983 and 984, of SEQ ID NO: 9:
983; P; and
984; P.

Advantageously, a proline residue at positions 983 and 984 increases the stability of the vaccine design.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, comprises the following amino acid residues at positions corresponding to residues 679, 680, 682, 983, and 984, of SEQ ID NO:9:
679; G;
680; S;
682; S;
983; P; and
984; P.

The Applicant has appreciated that polypeptides of the invention which comprise the above residue changes have better stability than polypeptides comprising the wild-type residues at these positions.

Optionally a polypeptide of the invention which comprises the amino acid sequence of SEQ ID NO: 2 (CoV_S_T2_36), or an amino acid which has at least 97%, 98%, or 99% amino acid identity over its entire length with the amino acid sequence of SEQ ID NO:2, does not include residues KFDEDDSEPVLKGVKLHYT at positions corresponding to amino acid residue positions 1252-1270 of SEQ ID NO:9.

The Applicant has appreciated that deletion of 19 amino acid residues from the C-terminus of the SARS-CoV-2 S protein at positions corresponding to residue positions 1252-1270 of SEQ ID NO: 9 (ER signal sequence) improves the expression of the designed polypeptide sequence.

According to the invention there is provided an isolated polypeptide which comprises CoV_S_T2_36 (omide) amino acid sequence (SEQ ID NO:2).

According to the invention there is provided an isolated polypeptide which comprises a coronavirus S protein with at least one, or all of the amino acid residues or deletions, at positions corresponding to the amino acid residue positions of SEQ ID NO:9, as shown in Table 6 below.

TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 679 | R | G |
| 680 | R | S |
| 682 | R | S |

Optionally there is provided an isolated polypeptide which comprises a coronavirus S protein which comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:9, and which comprises at least one, or all of the amino acid residues or deletions, at positions corresponding to the amino acid residue positions of SEQ ID NO:9, as shown in Table 6.

Optionally an isolated polypeptide according to the invention which comprises a coronavirus S protein comprises the following amino acid residues at positions corresponding to residues 984, and 985, of SEQ ID NO:9:
983; P; and
984; P.

Optionally an isolated polypeptide according to the invention which comprises a coronavirus S protein comprises the following amino acid residues at positions corresponding to residues 680, 681, 683, 984, and 985, of SEQ ID NO:9:
679; G;
680; S;
682; S;
983; P; and
984; P.

Optionally there is provided an isolated polypeptide which comprises a coronavirus S protein which comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:9 and which comprises the amino acid residues of SEQ ID NO:2, at positions corresponding to the amino acid residue positions of SEQ ID NO: 9, as shown in Table 5 below:

TABLE 5

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 983 | K | P |
| 984 | V | P |

Optionally there is provided an isolated polypeptide which comprises a coronavirus S protein which comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:9, and which comprises the amino acid residues of SEQ ID NO:2, at positions corresponding to the amino acid residue positions of SEQ ID NO: 9, as shown in Table 4 below:

TABLE 4

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 679 | R | G |
| 680 | R | S |
| 682 | R | S |
| 983 | K | P |
| 984 | V | P |

According to the invention there is also provided an isolated polypeptide which comprises a coronavirus S protein having an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:9, and which does not include residues KFDEDDSEPVLKGVKLHYT at positions corresponding to amino acid residue positions 1252-1270 of SEQ ID NO:9.

### Omicron Vaccine (SEQ ID NO:3)

There is also provided according to the invention an isolated polypeptide, which comprises the amino acid sequence of SEQ ID NO:3 (Omicron_vaccine), or an amino acid which has at least 99% amino acid identity over its entire length with the amino acid sequence of SEQ ID NO: 3.

>Omicron vaccine
(SEQ ID NO: 3)
Amino acid sequence:
MFVFLVLLPLVSSQCVNLTTRTQLPPAYTNSFTRGVYYPDKVFRSSVLH
STQDLFLPFFSNVTWFHVISGTNGTKRFDNPVLPFNDGVYFASIEKSNI
IRGWIFGTTLDSKTQSLLIVNNATNVVIKVCEFQFCNDPFLDHKNNKSW
MESEFRVYSSANNCTFEYVSQPFLMDLEGKQGNFKNLREFVFKNIDGYF
KIYSKHTPIIVREPEDLPQGFSALEPLVDLPIGINITRFQTLLALHRSY
LTPGDSSSGWTAGAAAYYCGYLQPRTFLLKYNENGTITDAVDCALDPLS
ETKCTLKSFTVEKGIYQTSNFRVQPTESIVRFPNITNLCPFDEVFNATR
FASVYAWNRKRISNCVADYSVLYNLAPFFTFKCYGVSPTKLNDLCFTNV
YADSFVIRGDEVRQIAPGQTGNIADYNYKLPDDFTGCVIAWNSNKLDSK
VSGNYNYLYRLFRKSNLKPFERDISTEIYQAGNKPCNGVAGFNCYFPLR
SYSFRPTYGVGHQPYRVVVLSFELLHAPATVCGPKKSTNLVKNKCVNFN
FNGLKGTGVLTESNKKFLPFQQFGRDIADTTDAVRDPQTLEILDITPCS
FGGVSVITPGTNTSNQVAVLYQGVNCTEVPVAIHADQLTPTWRVYSTGS
NVFQTRAGCLIGAEYVNNSYECDIPIGAGICASYQTQTKSHGSASSVAS
QSIIAYTMSLGAENSVAYSNNSIAIPTNFTISVTTEILPVSMTKTSVDC
TMYICGDSTECSNLLLQYGSFCTQLKRALTGIAVEQDKNTQEVFAQVKQ
IYKTPPIKYFGGFNFSQILPDPSKPSKRSFIEDLLFNKVTLADAGFIKQ
YGDCLGDIAARDLICAQKFKGLTVLPPLLTDEMIAQYTSALLAGTITSG
WTFGAGAALQIPFAMQMAYRFNGIGVTQNVLYENQKLIANQFNSAIGKI
QDSLSSTASALGKLQDVVNHNAQALNTLVKQLSSKFGAISSVLNDIFSR
LDPPEAEVQIDRLITGRLQSLQTYVTQQLIRAAEIRASANLAATKMSEC
VLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVI
GIVNNTVYDPLQPELDSFKEELDKYFKNHTSPDVDLGDISGINASVVNI
QKEIDRLNEVAKNLNESLIDLQELGKYEQYIKWPWYIWLGFIAGLIAIV
MVTIMLCCMTSCCSCLKGCCSCGSCC

Omicron_vaccine (SEQ ID NO:3) is the most phylogenetically similar to Omicron BA. 1C out of the reference sequences. Omicron_vaccine differs from BA. 1 by a double proline mutation at positions 983 and 984 of BA.1C, incorporated to assess the effect of such mutation on vaccine stability, and by deletion of the ER signal sequence, to assess the effect on protein expression. The amino acid differences of Omicron_vaccine (SEQ ID NO:3) from the Omicron BA. 1C S protein reference sequence (SEQ ID NO:9) are shown in Table 7 below:

TABLE 7

| | Omicron | |
|---|---|---|
| | BA. 1C | |
| | (SEQ ID | |
| Omicron BA. 1C | NO: 9) | |
| (SEQ ID NO: 9) | reference | Omicron_vaccine |
| residue position | residue | (SEQ ID NO: 3) |
| 983 | K | P |
| 984 | V | P |
| 1252-1270 | KFDEDD | — (deletion) |
| | SEPVLK | |
| | GVKLHY | |
| | T | |
| Total No of | — | — 21/1270 |
| differences from | | |
| reference | | |
| Percentage | — | — 98.35% |
| identity with the | | |
| reference | | |

The amino acid residues involved in stabilising the polypeptide design are in bold format. The deletion of amino acids corresponding to residues 1252-1270 of SEQ ID NO:9 are shown as “deletion”. The deletion of these residues promotes expression of the designed polypeptide sequence. The residue positions shown in the table correspond to the reside positions of both BA. 1C and Omicron_vaccine sequences.

Optionally a polypeptide of the invention, which comprises the amino acid sequence of SEQ ID NO: 3 (Omicron_vaccine), or an amino acid which has at least 99% amino acid identity over its entire length with the amino acid sequence of SEQ ID NO:3, comprises at least one, or both of the amino acid residues of SEQ ID NO:3, at positions corresponding to the amino acid residue positions of SEQ ID NO:9, as shown in Table 7 (replicated below without showing deletions):

TABLE 7

| | Omicron | |
|---|---|---|
| | BA.1C | |
| | (SEQ ID | |
| Omicron BA.1C | NO: 9) | |
| (SEQ ID NO: 9) | reference | Omicron_vaccine |
| residue position | residue | (SEQ ID NO: 3) |
| 983 | K | P |
| 984 | V | P |

The Applicant has appreciated that polypeptides of the invention which comprise the above residue changes have better stability than polypeptides comprising the wild-type residues at these positions.

Optionally a polypeptide of the invention, which comprises the amino acid sequence of SEQ ID NO: 3 (Omicron_vaccine), or an amino acid which has at least 99% amino acid identity over its entire length with the amino acid sequence of SEQ ID NO:3, does not include residues KFDEDDSEPVLKGVKLHYT at positions corresponding to amino acid residue positions 1252-1270 of SEQ ID NO:9.

The Applicant has appreciated that deletion of residues at positions corresponding to residue positions 1252-1270 of SEQ ID NO:9 (endoplasmic reticulum signal sequence) from polypeptides of the invention results in better expression of the polypeptide than polypeptides comprising such residues.

According to the invention there is provided an isolated polypeptide which comprises Omicron_vaccine amino acid sequence (SEQ ID NO:3).

The Applicant has found that such polypeptides elicit broadly neutralising immune responses to a panel of SARS-CoV-2 pseudoviruses, including Delta and omicron sub-strains BA. 1, BA.2, BA.2.12.1, and BA.4/5. As above, BA.4 and BA.5 are the dominant (as of April 2023) SARS-CoV-2 strains currently circulating in many countries around the world.

A polypeptide of the invention may include one or more conservative amino acid substitutions. Conservative amino acid substitutions are those substitutions that, when made, least interfere with the properties of the original polypeptide, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

The term “broadly neutralising immune response” is used herein to mean an immune response elicited in a subject that is sufficient to inhibit (i.e. reduce), neutralise or prevent infection, and/or progress of infection, of more than one strain and/or variant of SARS-CoV-2. Preferably, a broadly neutralising immune response is sufficient to inhibit, neutralise or prevent infection, and/or progress of infection of one or more variants of the Omicron strain of SARS-CoV-2. Optionally, a broadly neutralising immune response is sufficient to inhibit, neutralise or prevent infection, and/or progress of infection of one or more variants of the Delta strain of SARS-CoV-2. Optionally, a broadly neutralising immune response is sufficient to inhibit, neutralise or prevent infection, and/or progress of infection of one or more variants of the Alpha strain of SARS-CoV-2. Optionally, a broadly neutralising immune response is sufficient to inhibit, neutralise or prevent infection, and/or progress of infection of one or more variants of the Beta strain of SARS-CoV-2. Optionally, a broadly neutralising immune response is sufficient to inhibit, neutralise or prevent infection, and/or progress of infection of one or more variants of the Gamma strain of SARS-CoV-2. Optionally, a broadly neutralising immune response is sufficient to inhibit, neutralise or prevent infection, and/or progress of infection of one or more variants of the Wuhan strain of SARS-CoV-2. Optionally a broadly neutralising immune response is sufficient to inhibit, neutralise or prevent infection, and/or progress of infection, of most or all different viral strains of the SARS-CoV-2 virus. Optionally a broadly neutralising immune response is sufficient to inhibit, neutralise or prevent infection, and/or progress of infection, of more than one type of β-coronavirus SARS-CoV-2 variant of concern (VOC), or variant of interest (VOI), for example more than one of an alpha, beta, gamma, delta, omicron SARS-CoV-2 virus.

Optionally, a broadly neutralising immune response is sufficient to inhibit, neutralise or prevent infection, and/or progress of infection of a virus within the coronavirus family. Optionally a broadly neutralising immune response is sufficient to inhibit, neutralise or prevent infection, and/or progress of infection, of more than one type of β-coronavirus (for example, SARS-CoV, and SARS-CoV-2). Optionally a broadly neutralising immune response is sufficient to inhibit, neutralise or prevent infection, and/or progress of infection, of more than one type of β-coronavirus within the same β-coronavirus lineage (for example, more than one type of β-coronavirus within the subgenus Sarbecovirus, such as SARS-CoV, SARS-CoV-2, and Bat SL-CoV-WIV1). Optionally a broadly neutralising immune response is sufficient to inhibit, neutralise or prevent infection, and/or progress of infection, of coronaviruses of different β-coronavirus lineages, such as lineage B (for example, SARS-CoV, and SARS-CoV-2) and lineage C (for example, MERS-COV). Optionally a broadly neutralising immune response is sufficient to inhibit, neutralise or prevent infection, and/or progress of infection, of most or all different β-coronaviruses. Optionally a broadly neutralising immune response is sufficient to inhibit, neutralise or prevent infection, and/or progress of infection, of most or all different viruses of the coronavirus family.

The immune response may be humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

Optionally a polypeptide of the invention is a glycosylated polypeptide.

### Nucleic Acid Molecules

According to the invention there is provided an isolated nucleic acid molecule encoding a polypeptide according to the invention, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to a nucleic acid molecule of the invention encoding a polypeptide of the invention, or the complement thereof.

According to the invention there is provided an isolated nucleic acid molecule comprising a nucleotide sequence encoding an isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO: 1, or the complement thereof.

According to the invention there is provided an isolated nucleic acid molecule comprising a nucleotide sequence encoding an isolated polypeptide sequence comprising the amino acid sequence of SEQ ID NO:1, or the complement thereof.

Optionally, there is provided an isolated nucleic acid molecule comprising a nucleotide sequence encoding an isolated polypeptide which comprises an amino acid sequence of SEQ ID NO: 2 (CoV_S_T2_36), or an amino acid sequence which has at least 97%, 98%, or 99% amino acid identity over its entire length with the amino acid sequence of SEQ ID NO:2, or the complement thereof.

According to the invention there is provided an isolated polynucleotide molecule comprising a nucleotide sequence encoding an isolated polypeptide which comprises the amino acid sequence of SEQ ID NO:2, or the complement thereof.

According to the invention there is provided an isolated nucleic acid molecule comprising a nucleotide sequence encoding an isolated polypeptide which comprises an amino acid sequence of SEQ ID NO:3 (Omicron_Vaccine), or an amino acid sequence which has at 99% amino acid identity over its entire length with the amino acid sequence of SEQ ID NO:3, or the complement thereof.

According to the invention there is provided an isolated polynucleotide molecule comprising a nucleotide sequence encoding the amino acid sequence of SEQ ID NO:3, or the complement thereof.

mRNA Molecules

We have appreciated that advantageous immunogenic properties (for example, increased antibody response and/or increased breadth of immune response) are provided with mRNA immunogens encoding coronavirus spike proteins according to the invention.

According to the invention there is also provided an isolated mRNA encoding a polypeptide comprising an amino acid sequence of a coronavirus spike protein CoV_S_T2_35 (SEQ ID NO: 1)

According to the invention there is also provided an isolated mRNA encoding a polypeptide comprising an amino acid sequence of a coronavirus spike protein CoV_S_T2_36 (SEQ ID NO: 2)

According to the invention there is also provided an isolated mRNA encoding a polypeptide comprising an amino acid sequence of a coronavirus spike protein Omicron_Vaccine (SEQ ID NO: 3).

According to the invention there is provided an isolated mRNA molecule comprising an RNA sequence encoding a polypeptide according to the invention, or the complement thereof.

There is also provided according to the invention an isolated mRNA molecule comprising an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to an mRNA molecule of the invention encoding a polypeptide of the invention, or the complement thereof.

According to the invention there is provided an isolated mRNA molecule comprising an RNA sequence encoding an isolated polypeptide which comprises an amino acid sequence of SEQ ID NO: 1 (CoV_S_T2_35), or an amino acid sequence which has at least 97%, 98%, or 99% amino acid identity over its entire length with the amino acid sequence of SEQ ID NO: 1, or the complement thereof.

Optionally there is provided an isolated mRNA molecule comprising an RNA sequence encoding an isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO:1, which comprises the amino acid residues of SEQ ID NO: 1, at positions corresponding to the amino acid residue positions of SEQ ID NO:8, as shown in Table 3 below:

TABLE 3

| | DeltaC (SEQ | |
|---|---|---|
| DeltaC (SEQ ID | ID NO: 8) | CoV_S_T2_35 |
| NO: 8) residue | reference | (deom) SEQ ID |
| position | residue | NO: 1) |
| 680 | R | G |
| 681 | R | S |
| 683 | R | S |

Optionally there is provided an isolated mRNA molecule comprising an RNA sequence encoding an isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO:1, which comprises the following amino acid residues at positions corresponding to residues 984, and 985, of SEQ ID NO: 8:
984; P; and
985; P.

Optionally there is provided an isolated mRNA molecule comprising an RNA sequence encoding an isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO:1, which comprises the following amino acid residues at positions corresponding to residues 680, 681, 683, 984, and 985, of SEQ ID NO:8:
680; G;
681; S;
683; S;
984; P; and
985; P.

Optionally there is provided an isolated mRNA molecule comprising an RNA sequence encoding an isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO: 1, which does not include residues KFDEDDSEPVLKGVKLHYT at positions corresponding to amino acid residue positions 1253-1271 of SEQ ID NO:8.

According to the invention there is provided an isolated mRNA molecule comprising an RNA sequence encoding an isolated polypeptide which comprises a coronavirus S protein with at least one, or all of the amino acid residues, at positions corresponding to the amino acid residue positions of SEQ ID NO:8, as shown in Table 3 below:

TABLE 3

| DeltaC (SEQ | DeltaC (SEQ | |
|---|---|---|
| ID NO: 8) | ID NO: 8) | CoV_S_T2_35 |
| residue | reference | (deom) SEQ ID |
| position | residue | NO: 1) |
| 680 | R | G |
| 681 | R | S |
| 683 | R | S |

Optionally, there is provided an isolated mRNA molecule comprising an RNA sequence encoding an isolated polypeptide which comprises an amino acid sequence of SEQ ID NO:2 (CoV_S_T2_36), or an amino acid sequence which has at least 97%, 98%, or 99% amino acid identity over its entire length with the amino acid sequence of SEQ ID NO:2, or the complement thereof.

Optionally there is provided an isolated mRNA molecule comprising an RNA sequence encoding an isolated polypeptide which comprises an amino acid sequence of SEQ ID NO:2 (CoV_S_T2_36), or an amino acid sequence which has at least 97%, 98%, or 99% amino acid identity over its entire length with the amino acid sequence of SEQ ID NO:2, which comprises the amino acid residues of SEQ ID NO:2, at positions corresponding to the amino acid residue positions of SEQ ID NO:9, as shown in Table 6 below:

TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 679 | R | G |
| 680 | R | S |
| 682 | R | S |

Optionally there is provided an isolated mRNA molecule comprising an RNA sequence encoding an isolated polypeptide which comprises an amino acid sequence of SEQ ID NO:2 (CoV_S_T2_36), or an amino acid sequence which has at least 97%, 98%, or 99% amino acid identity over its entire length with the amino acid sequence of SEQ ID NO:2, which comprises the following amino acid residues at positions corresponding to residues 983, and 984, of SEQ ID NO: 9:
983; P; and
984; P.

Optionally there is provided an isolated mRNA molecule comprising an RNA sequence encoding an isolated polypeptide which comprises an amino acid sequence of SEQ ID NO:2 (CoV_S_T2_36), or an amino acid sequence which has at least 97%, 98%, or 99% amino acid identity over its entire length with the amino acid sequence of SEQ ID NO:2, which comprises the following amino acid residues at positions corresponding to residues 679, 680, 682, 983, and 984, of SEQ ID NO:9:
679; G;
680; S;
682; S;
983; P; and
984; P.

Optionally there is provided an isolated mRNA molecule comprising an RNA sequence encoding an isolated polypeptide which comprises an amino acid sequence of SEQ ID NO:2 (CoV_S_T2_36), or an amino acid sequence which has at least 97%, 98%, or 99% amino acid identity over its entire length with the amino acid sequence of SEQ ID NO:2, which does not include residues KFDEDDSEPVLKGVKLHYT at positions corresponding to amino acid residue positions 1252-1270 of SEQ ID NO:9.

According to the invention there is provided an isolated mRNA molecule comprising an RNA sequence encoding an isolated polypeptide which comprises a coronavirus S protein with at least one, or all of the amino acid residues, at positions corresponding to the amino acid residue positions of SEQ ID NO:9, as shown in Table 6 below:

TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 679 | R | G |
| 680 | R | S |
| 682 | R | S |

According to the invention there is provided an isolated mRNA molecule comprising an RNA sequence encoding an isolated polypeptide which comprises an amino acid sequence of SEQ ID NO: 3 (Omicron_Vaccine), or an amino acid sequence which has at 99% amino acid identity over its entire length with the amino acid sequence of SEQ ID NO:3, or the complement thereof.

Optionally there is provided an isolated mRNA molecule comprising an RNA sequence encoding an isolated polypeptide which comprises an amino acid sequence of SEQ ID NO:3 (Omicron_vaccine), or an amino acid sequence which has at least 99% amino acid identity over its entire length with the amino acid sequence of SEQ ID NO:2, which comprises the following amino acid residues at positions corresponding to residues 983, and 984, of SEQ ID NO: 9:
983; P; and
984; P.

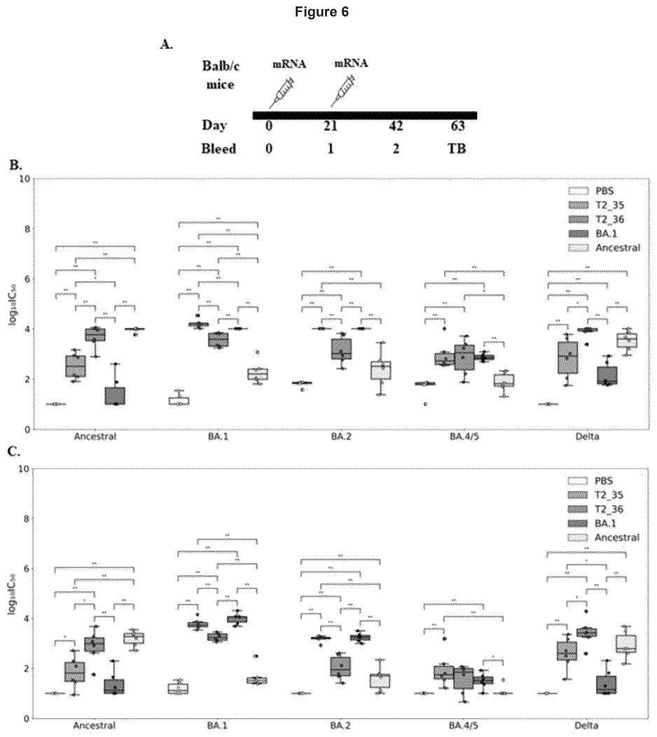

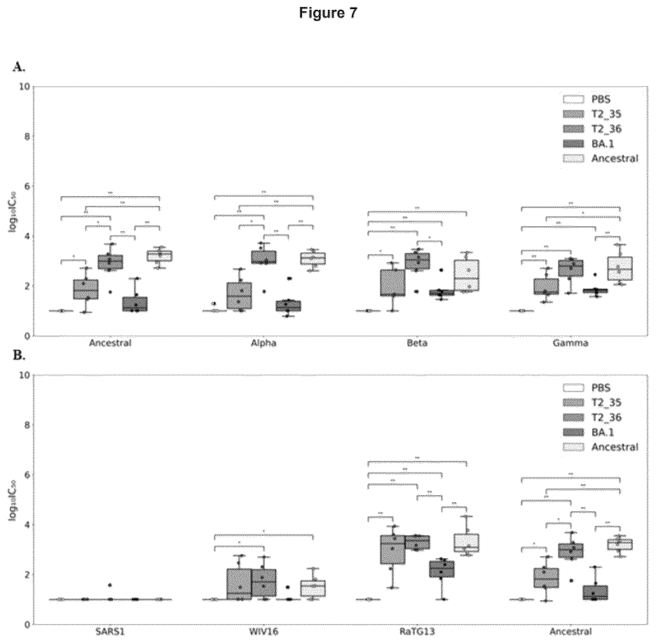

We have found that immunisation of mice with nucleic acid (in particular, mRNA) encoding SARS2 S protein designs of the invention induces production of antibodies that are able to bind and neutralise coronavirus pseudoviruses expressing spike proteins (see Example 3/FIGS. 6 and 7). In particular, immunisation of mice with a mRNA vaccine comprising nucleic acid encoding the CoV_S_T2_35(Deom) mRNA vaccine design of the invention (SEQ ID NO: 1) elicited broadly neutralising antibody response to Wuhan, Alpha, Beta, Gamma, Delta, and Omicron BA. 1, BA.2, BA.2.12.1, and BA.4/5 SARS-CoV-2 (FIGS. 6B and 6C, FIG. 7). Similarly, immunisation of mice with a mRNA vaccine comprising nucleic acid encoding the CoV_S_T2_36 (Omide) mRNA vaccine design of the invention (SEQ ID NO:2) elicited broadly neutralising antibody response to BA.1, BA.2, BA.2.12.1, and BA.4/5 sub-strains of the Omicron strain, as well as the Wuhan, Alpha, Beta, Gamma, and Delta, strains of SARS-CoV-2 (FIGS. 6B and 6C, FIG. 7). Immunisation of mice with a mRNA vaccine comprising nucleic acid encoding the Omicron_vaccine (Deom) design of the invention (SEQ ID NO:3) elicited broadly neutralising antibody response to the Beta, Gamma, BA. 1, BA.2, BA.4/5, and Delta strain of SARS-CoV-2.

### Sequence Identity

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from http://bitincka.com/ledion/matgat), Gap (Needleman & Wunsch, 1970, J. Mol. Biol. 48:443-453), FASTA (Altschul et al., 1990, J. Mol. Biol. 215:403-410; program available from http://www.ebi.ac.uk/fasta), Clustal W 2.0 and X 2.0 (Larkin et al., 2007, Bioinformatics 23:2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

### Corresponding Positions

Sequences described herein include reference to an amino acid sequence comprising an amino acid residue “at a position corresponding to an amino acid residue position” of another sequence. Such corresponding positions may be identified, for example, from an alignment of the sequences using a sequence alignment method described herein, or another sequence alignment method known to the person of ordinary skill in the art.

### Vectors and Vaccines

There is also provided according to the invention a vector comprising a nucleic acid molecule encoding a polypeptide of the invention.

Optionally a vector of the invention comprises a nucleic acid molecule encoding a polypeptide of the invention which comprises an amino acid sequence of SEQ ID NO: 1, or an amino acid sequence which has at least 97%, 98%, or 99% amino acid identity over its entire length with the amino acid sequence of SEQ ID NO:1.

Optionally a vector of the invention comprises a polynucleotide molecule comprising a nucleotide sequence encoding the amino acid sequence of SEQ ID NO: 1, or the complement thereof.

Optionally a vector of the invention comprises a nucleic acid molecule encoding a polypeptide of the invention which comprises an amino acid sequence of SEQ ID NO: 2, or an amino acid sequence which has at least 97%, 98%, or 99% amino acid identity over its entire length with the amino acid sequence of SEQ ID NO:2.

Optionally a vector of the invention comprises a polynucleotide molecule comprising a nucleotide sequence encoding the amino acid sequence of SEQ ID NO:2, or the complement thereof.

Optionally a vector of the invention comprises a nucleic acid molecule encoding a polypeptide of the invention which comprises an amino acid sequence of SEQ ID NO: 1, or an amino acid sequence which has at least 99% amino acid identity over its entire length with the amino acid sequence of SEQ ID NO:3.

Optionally a vector of the invention comprises a nucleic acid molecule encoding a polypeptide of the invention which comprises an amino acid sequence of SEQ ID NO: 3, or an amino acid sequence which has at least 99% amino acid identity over its entire length with the amino acid sequence of SEQ ID NO:3.

Optionally a vector of the invention comprises a polynucleotide molecule comprising a nucleotide sequence encoding the amino acid sequence of SEQ ID NO:3, or the complement thereof.

Optionally, a vector of the invention comprises a nucleic acid molecule of the invention encoding a designed coronavirus S protein of the invention.

Optionally a vector of the invention further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast or insect cells.

Optionally the vector is a vaccine vector.

Optionally the vector is a viral vaccine vector, a bacterial vaccine vector, an RNA vaccine vector, or a DNA vaccine vector.

A nucleic acid molecule of the invention may comprise a DNA or an RNA molecule. For embodiments in which the nucleic acid comprises an RNA molecule, it will be appreciated that the nucleic acid sequence of the nucleic acid will be the same as that recited in the respective SEQ ID, or the complement thereof, but with each ‘T’ nucleotide replaced by ‘U’.

For embodiments in which the nucleic acid molecule comprises an RNA molecule, it will be appreciated that the molecule may comprise an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, a polynucleotide sequence encoding a polypeptide sequence of any of SEQ ID NOs: 1, 2, or 3, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 1, 2, or 3 in which each ‘T’ nucleotide is replaced by ‘U’, or the complement thereof.

Viral vaccine vectors use live viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4+ T cell), and cytotoxic T lymphocyte (CTL, CD8+ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles), Alphavirus (e.g. SFV, Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector. Optionally the viral vaccine vector is a pURVac vaccine vector, a derivative of a DNA vaccine vector.

Adenoviruses are by far the most utilised and advanced viral vectors developed for SARS2 vaccines. They are non-enveloped double-stranded DNA (dsDNA) viruses with a packaging capacity of up to 7.5 kb of foreign genes. Almost all SARS2 adenovirus based vaccines have been engineered for the expression of the SARS2 S protein or the RBD subunit. Recombinant Adenovirus vectors are widely used because of their high transduction efficiency, high level of transgene expression, and broad range of viral tropism. These vaccines are highly cell specific, highly efficient in gene transduction, and efficient at inducing an immune response. Adenovirus vaccines are effective at triggering and priming T cells, leading to long term and high level of antigenic protein expression and therefore long lasting protection. AZD1222 (manufactured by AstraZeneca) vaccine construct comprises a recombinant adenoviral vector vaccine encoding the SARS2 S protein. The recombinant adenovirus genome comprises SARS2 S gene at the E1 locus.

Optionally a vaccine of the invention (optionally a nucleic acid or polypeptide of the invention) is administered as part of a heterologous prime-boost regimen, for example using an heterologous DNA prime/MVA boost regimen.

Optionally a method of inducing an immune response to a coronavirus in a subject, or a method of immunising a subject against a coronavirus, according to the invention comprises administering a nucleic acid of the invention, a vector of the invention, or a pharmaceutical composition of the invention, wherein the nucleic acid, vector, or pharmaceutical composition is administered as part of a heterologous prime boost regimen.

Optionally the heterologous prime boost regimen comprises a prime with a DNA vector of the invention followed by a boost with an MVA vector of the invention.

Optionally the DNA prime comprises administration of a DNA vaccine vector comprising a nucleic acid molecule of the invention, and the MVA boost comprises administration of an MVA vector comprising a nucleic acid molecule of the invention, optionally wherein the nucleic acid molecule of the invention of the DNA vaccine vector encodes the same amino acid sequence as the nucleic acid molecule of the invention of the MVA vector. Optionally the nucleic acid molecule of the invention of the DNA vaccine vector encodes a different amino acid sequence as the nucleic acid molecule of the invention of the MVA vector.

For example, a nucleic acid molecule (optionally a DNA molecule) encoding a designed S protein sequence T2_29 polypeptide of the invention (SEQ ID NO:29; CoV_S_T2_29+Q498R+dER); (CoV_S_T2_29+Q498R—SEQ ID NO:28); or (CoV_S_T2_29—SEQ ID NO:27) may be administered as part of an heterologous prime-boost vaccination using an MVA boost. As shown in Example 3 below, a prime with DNA vector comprising DNA encoding amino acid sequence of SEQ ID NO:27, 28, or 29, followed by a boost with an MVA vector comprising nucleic acid encoding amino acid sequence of SEQ ID NO: 29, induced broad neutralising response against all the VOCs tested—at least two-fold better neutralising response against Beta, Gamma, Delta, and Omicron VOCs in comparison to WTdER after three doses of DNA vaccine.

Optionally the prime with a DNA vector of the invention may comprise administration of the DNA vector once, twice, or three times, prior to the MVA boost.

The MVA boost may be administered at least a day, at least a week, or at least two, three, four, five, six, or seven weeks, after the final administration of the DNA vector.

There is also provided according to the invention a kit comprising a DNA vaccine vector which comprises a nucleic acid molecule of the invention, and an MVA vector which comprises a nucleic acid molecule of the invention, optionally wherein the nucleic acid molecule of the invention of the DNA vaccine vector encodes the same amino acid sequence as the nucleic acid molecule of the invention of the MVA vector.

Optionally the nucleic acid molecule of the invention of the DNA vaccine vector encodes a designed S protein sequence T2_29 polypeptide of the invention (SEQ ID NO:29—CoV_S_T2_29+Q498R+dER; CoV_S_T2_29+Q498R—SEQ ID NO:28; or CoV_S_T2_29—SEQ ID NO:27), and the nucleic acid molecule of the invention of the MVA vector encodes an amino acid sequence of SEQ ID NO:29.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a Kpnl restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the vector is a pEVAC-based expression vector.

The pEVAC vector has proven to be a highly versatile expression vector for generating viral pseudotypes as well as direct DNA vaccination of animals and humans.

Optionally the vector is a pURVAC vector.

The terms “polynucleotide” and “nucleic acid” are used interchangeably herein.

A polynucleotide (or nucleic acid) of the invention may comprise a DNA molecule.

The or each polynucleotide (or nucleic acid) of a pharmaceutical composition or a vector, of the invention may comprise a DNA molecule.

A vector of the invention may be a DNA vector.

The or each vector of a pharmaceutical composition of the invention may be a DNA vector.

A polynucleotide (or nucleic acid) of the invention, or a polynucleotide (or nucleic acid) of a pharmaceutical composition, or a vector, of the invention, may be provided as part of a DNA vaccine.

There is also provided according to the invention a DNA vaccine which comprises a polynucleotide (or nucleic acid) of the invention, a vector of the invention, or a pharmaceutical composition of the invention which comprises one or more polynucleotides (or nucleic acids), wherein the or each polynucleotide (or nucleic acid) is a DNA molecule.

Optionally the, or each, vector is an RNA vaccine vector.

A polynucleotide (or nucleic acid) of the invention may comprise an RNA molecule.

The or each polynucleotide (or nucleic acid) of a pharmaceutical composition, or a vector, of the invention may comprise an RNA molecule.

The or each vector of a pharmaceutical composition or a combined preparation of the invention may be an RNA vector.

A polynucleotide (or nucleic acid) of the invention, or a polynucleotide (or nucleic acid) of a pharmaceutical composition, or a vector, of the invention, may be provided as part of an RNA vaccine.

There is also provided according to the invention an RNA vaccine which comprises a polynucleotide (or nucleic acid) of the invention, a vector of the invention, or a pharmaceutical composition of the invention which comprises one or more polynucleotides (or nucleic acids), wherein the or each polynucleotide (or nucleic acid) is an RNA molecule.

A polynucleotide (or nucleic acid) of the invention may comprise an mRNA molecule.

The or each polynucleotide (or nucleic acid) of a pharmaceutical composition, or a vector, of the invention may comprise an mRNA molecule.

A vector of the invention may be an mRNA vector.

Optionally the, or each vaccine vector is an mRNA vaccine vector.

The or each vector of a pharmaceutical composition of the invention may be an mRNA vector.

A polynucleotide (or nucleic acid), or mRNA molecule, of the invention, or a polynucleotide (or nucleic acid) of a pharmaceutical composition, or a vector, of the invention, may be provided as part of an mRNA vaccine.

There is also provided according to the invention an mRNA vaccine, which comprises an mRNA of the invention, or an mRNA vaccine vector of the invention, encapsulated in a lipid nanoparticle (LNP).

There is also provided according to the invention an mRNA vaccine which comprises a polynucleotide (or nucleic acid) of the invention, a vector of the invention, or a pharmaceutical composition of the invention which comprises one or more polynucleotides (or nucleic acids), wherein the or each polynucleotide (or nucleic acid) comprises an mRNA molecule. Messenger RNA (mRNA) vaccines are a new form of vaccine (recently reviewed in Pardi et al., Nature Reviews Drug Discovery Volume 17, pages 261-279 (2018); Wang et al., Molecular Cancer (2021) 20:33: mRNA vaccine: a potential therapeutic strategy). The first mRNA vaccines to be approved for use were BNT162b2 (manufactured by Pfizer) and mRNA-1273 (manufactured by Moderna) during the COVID-19 pandemic. mRNA vaccines have a unique feature of temporarily promoting the expression of antigen (typically days). The expression of the exogenous antigen is controlled by the lifetime of encoding mRNA, which is regulated by cellular degradation pathways. While this transient nature of protein expression requires repeated administration for the treatment of genetic diseases and cancers, it is extremely beneficial for vaccines, where prime or prime-boost vaccination is sufficient to develop highly specific adaptive immunity without any exposure to the contagion.

mRNA based vaccines trigger an immune response after the synthetic mRNA which encodes viral antigens transfects human cells. The cytosolic mRNA molecules are then translated by the host's own cellular machinery into specific viral antigens. These antigens may then be presented on the cell surface where they can be recognised by immune cells, triggering an immune response.

The structural elements of a vaccine vector mRNA molecule are similar to those of natural mRNA, comprising a 5′ cap, 5′ untranslated region (UTR), coding region (for example, comprising an open reading frame encoding a polypeptide of the invention), 3′ UTR, and a poly(A) tail. The 5′ UTR (also known as a leader sequence, transcript leader, or leader RNA) is the region of an mRNA that is directly upstream from the initiation codon. This region is important for the regulation of translation of a transcript. In many organisms, the 5′ UTR forms complex secondary structure to regulate translation. The 5′ UTR begins at the transcription start site and ends one nucleotide (nt) before the initiation sequence (usually AUG) of the coding region. In eukaryotes, the length of the 5′ UTR tends to be anywhere from 100 to several thousand nucleotides long. The differing sizes are likely due to the complexity of the eukaryotic regulation which the 5′ UTR holds as well as the larger pre-initiation complex that must form to begin translation. The eukaryotic 5′ UTR contains the Kozak consensus sequence (ACCAUG (initiation codon underlined), which contains the initiation codon AUG. An elongated Kozak sequence may be used: GCCACCAUG (initiation codon underlined).

The 5′ and 3′ UTR elements flanking the coding sequence profoundly influence the stability and translation of mRNA, both of which are critical concerns for vaccines. These regulatory sequences can be derived from viral or eukaryotic genes and greatly increase the half-life and expression of therapeutic mRNAs. For example, a 5′UTR of an mRNA of the invention may comprise, with an initiation codon of the mRNA, a Kozak consensus sequence, or an elongated Kozak sequence. Optionally a 5′UTR of an mRNA of the invention comprises the following sequence: GGAGACGCCACC immediately upstream of an initiation codon sequence.

A 5′ cap structure is required for efficient protein production from mRNA. Various versions of 5′ caps can be added during or after the transcription reaction using a vaccinia virus capping enzyme, or by incorporating synthetic cap or anti-reverse cap analogues (see Pardi et al., supra). Anti-Reverse Cap Analog (ARCA) is a cap analog used during in vitro transcription for the generation of capped transcripts. ARCA is modified in a way that ensures incorporation in the forward orientation only. Anti-Reverse Cap Analog (ARCA) is a modified cap analog in which the 3′ OH group (closer to m⁷G) is replaced with —OCH₃:

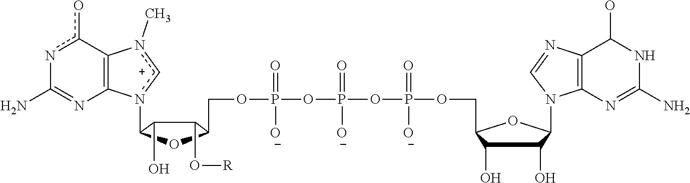

Conventional Cap Analog: R═H, m⁷G(5′)pppG;
ARCA: R═CH₃, 3′-0-Me-m⁷G(5′)pppG

Because of this substitution, the RNA polymerase can only initiate transcription with the remaining hydroxyl group thus forcing ARCA incorporation in the forward orientation. As a result, unlike transcripts synthesized with conventional cap analog, 100% of the transcripts synthesized with ARCA at the 5′ end are translatable leading to a strong stimulatory effect on translation.

The poly(A) tail also plays an important regulatory role in mRNA translation and stability; thus, an optimal length of poly(A) must be added to mRNA either directly from the encoding DNA template or by using poly(A) polymerase (see Pardi et al., supra). An example of a suitable length of poly(A) tail is poly(~A₁₂₀).

The codon usage additionally has an impact on protein translation. Replacing rare codons with frequently used synonymous codons that have abundant cognate tRNA in the cytosol is a common practice to increase protein production from mRNA. Enrichment of G: C content constitutes another form of sequence optimization that has been shown to increase steady-state mRNA levels in vitro and protein expression in vivo (see Pardi et al., supra).

Two major types of RNA are currently studied as vaccines: non-replicating mRNA and virally derived, self-amplifying RNA. While both types of vaccines share a common structure in mRNA constructs, self-amplifying RNA vaccines contain additional sequences in the coding region for RNA replication, including RNA-dependent RNA polymerases.

BNT 162b2 vaccine construct comprises a lipid nanoparticle (LNP) encapsulated mRNA molecule encoding trimerised full-length SARS2 S protein with a PP mutation (at residue positions 986-987). The mRNA is encapsulated in 80 nm ionizable cationic lipid nanoparticles.

mRNA-1273 vaccine construct is also based on an LNP vector, but the synthetic mRNA encapsulated within the lipid construct encodes the full-length SARS2 S protein.

U.S. Pat. No. 10,702,600 B1 (ModernaTX) describes betacoronavirus mRNA vaccines, including suitable LNPs for use in such vaccines.

A nucleic acid vaccine (for example, a mRNA) of the invention may be formulated in a lipid nanoparticle.

mRNA vaccines have several advantages in comparison with conventional vaccines containing inactivated (or live attenuated) disease-causing organisms. Firstly, mRNA-based vaccines can be rapidly developed due to design flexibility and the ability of the constructs to mimic antigen structure and expression as seen in the course of a natural infection. mRNA vaccines can be developed within days or months based on sequencing information from a target virus, while conventional vaccines often take years and require a deep understanding of the target virus to make the vaccine effective and safe. Secondly, these novel vaccines can be rapidly produced. Due to high yields from in vitro transcription reactions, mRNA production can be rapid, inexpensive and scalable. Thirdly, vaccine risks are low. mRNA does not contain infectious viral elements that pose risks for infection and insertional mutagenesis. Anti-vector immunity is also avoided as mRNA is the minimally immunogenic genetic vector, allowing repeated administration of the vaccine. The challenge for effective application of mRNA vaccines lies in cytosolic delivery. mRNA isolates are rapidly degraded by extracellular

RNases and cannot penetrate cell membranes to be transcribed in the cytosol. However, efficient in vivo delivery can be achieved by formulating mRNA into carrier molecules, allowing rapid uptake and expression in the cytoplasm. To date, numerous delivery methods have been developed including lipid-, polymer-, or peptide-based delivery, virus-like replicon particle, cationic nanoemulsion, naked mRNAs, and dendritic cell-based delivery (each reviewed in Wang et al., supra). Decationic lipid nanoparticle (LNP) delivery is the most appealing and commonly used mRNA vaccine delivery tool.

Exogenous mRNA may be highly immunostimulatory. Single-stranded RNA (ssRNA) molecules are considered a pathogen associated molecular pattern (PAMP), and are recognised by various Toll-like receptors (TLR) which elicit a pro-inflammatory reaction.

Although a strong cellular and humoral immune response is desirable in response to vaccination, the innate immune reaction elicited by exogenous mRNA may cause undesirable side-effects in the subject. The U-rich sequence of mRNA is a key element to activate TLR (Wang et al., supra). Additionally, enzymatically synthesised mRNA preparations contain double stranded RNA (dsRNA) contaminants as aberrant products of the in vitro transcription (IVT) process. dsRNA is a potent PAMP, and elicits downstream reactions resulting in the inhibition of translation and the degradation of cellular mRNA and ribosomal RNA (Pardi et al., supra). Thus, the mRNA may suppress antigen expression and thus reduce vaccine efficacy.

Studies over the past decade have shown that the immunostimulatory effect of mRNA can be shaped by the purification of IVT mRNA, the introduction of modified nucleosides, complexing the mRNA with various carrier molecules (Pardi et al., supra), adding poly(A) tails or optimising mRNA with GC-rich sequence (Wang et al., supra). Chemical modification of uridine is a common approach to minimise the immunogenicity of foreign mRNA. Incorporation of pseudouridine (ψ) and N1-methylpseudouridine (m1ψ) to IVT mRNA prevents TLR activation and other innate immune sensors, thus reducing pro-inflammatory signalling in response to the exogenous mRNA. Such nucleoside modification also suppresses recognition of dsRNA species (Pardi et al., supra) and can reduce innate immune sensing of exogenous mRNA translation (Hou et al. Nature Reviews Materials, 2021, https://doi.org/10.1038/s41578-021-00358-0).

Other nucleoside chemical modifications include, but are not limited to, 5-methylcytidine (m5C), 5-methyluridine (m5U), N1-methyladenosine (m1A), N6-methyladenosine (m6A), 2-thiouridine (s2U), and 5-methoxyuridine (5moU) (Wang et al., supra).

The IVT mRNA molecules used in the mRNA-1273 and BNT162b2 COVID-19 vaccines were prepared by replacing uridine with m1ψ, and their sequences were optimized to encode a stabilized pre-fusion spike protein with two pivotal proline substitutions (Hou et al., supra). However, CureVac's mRNA vaccine candidate, CVnCoV, uses unmodified nucleosides and relies on a combination of mRNA sequence alterations to allow immune evasion without affecting the expressed protein. Firstly, CVnCoV has a higher GC content (63%) than rival vaccines (BNT162b2 has 56%) and the original SARS-CoV-2 virus itself (37%). Secondly, the vaccine comprises C-rich motifs which bind to poly(C)-binding protein, enhancing both the stability and expression of the mRNA. A further modification of CVnCoV is that it contains a histone stem-loop sequence as well as a poly(A) tail, to enhance the longevity and translation of the mRNA (Hubert, B., 2021. The CureVac Vaccine, and a brief tour through some of the wonders of nature. URL https://berthub.eu/articles/posts/curevac-vaccine-and-wonders-of-biology/. (accessed 15.09.21). However, the vaccine had disappointing results from phase III clinical trials, which experts assert are down to the decision not to incorporate chemically modified nucleosides into the mRNA sequence. Nonetheless, CureVac and Acuitas Therapeutics delivered erythropoietin (EPO)-encoding mRNA, which has rich GC codons, to pigs with lipid nanoparticles (LNPs). Their results indicated EPO-related responses were elicited without immunogenicity (Wang et al., supra), suggesting that there is still scope for unmodified mRNA nucleoside-based vaccines. RNA or mRNA of a polynucleotide (or nucleic acid) of the invention, or of a polynucleotide (or nucleic acid) of a pharmaceutical composition, a combined preparation, a vector, or a vaccine, of the invention may be produced by in vitro transcription (IVT).

Optionally an IVT mRNA of the invention comprises a polyadenylation (poly(A)) tail downstream of an open reading frame (ORF) encoding the polypeptide.

A polynucleotide (or nucleic acid) or mRNA molecule of the invention, or a polynucleotide (or nucleic acid) or mRNA molecule of a pharmaceutical composition, a vector, or a vaccine, of the invention may comprise one or more modified nucleosides.

The one or more modified nucleosides may be present in DNA or RNA of a polynucleotide (or nucleic acid) of the invention, or of a polynucleotide (or nucleic acid) of a pharmaceutical composition, a vector, or a vaccine, of the invention.

Optionally, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine, 5-methylcytosine, N1-methyladenosine, N6-methyladenosine. In some embodiments, the chemical modification is in the 5-position of the uracil. In some embodiments, the chemical modification is a N1-methylpseudouridine. In some embodiments, the chemical modification is a N1-ethylpseudouridine.

For example, an RNA or an mRNA of a polynucleotide (or nucleic acid) of the invention, or of a polynucleotide (or nucleic acid) of a pharmaceutical composition, a vector, or a vaccine, of the invention may comprise one or more of the following modified nucleosides:
pseudouridine (ψ);
N1-methylpseudouridine (m1ψ) 5-methylcytidine (m5C) 5-methyluridine (m5U) N1-methyladenosine (m1A)

N6-methyladenosine (m6A) 2-thiouridine (s2U) 5-methoxyuridine (5moU)

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

The polynucleotide (or nucleic acid) may contain from about 1% to about 100% modified nucleotides (or nucleosides) (either in relation to overall nucleotide content, or in relation to one or more types of nucleotide (or nucleoside), i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

Optionally at least 50% of the uridines in the ORF have been modified.

Optionally at least 50% of the uridines in the ORF have been modified to m1ψ.

Optionally a polynucleotide (or nucleic acid), or mRNA molecule, of the invention, or of a polynucleotide (or nucleic acid), or mRNA molecule, of a pharmaceutical composition, a vector, or a vaccine, of the invention, comprises an RNA molecule in which the nucleic acid sequence of the polynucleotide (or nucleic acid) is the same as that recited in the respective SEQ ID, or the complement thereof, but with each ‘U’ replaced by m1ψ.

Optionally a polynucleotide (or nucleic acid), or mRNA molecule, of the invention, or of a polynucleotide (or nucleic acid), or mRNA molecule, of a pharmaceutical composition, a vector, or a vaccine, of the invention, comprises an mRNA molecule in which the nucleic acid sequence of the polynucleotide is the same as that recited in the respective SEQ ID, or the complement thereof, but with each ‘U’ replaced by m1ψ.

Optionally a polynucleotide (or nucleic acid), or mRNA molecule, of the invention, or of a polynucleotide (or nucleic acid), or mRNA molecule, of a pharmaceutical composition, a vector, or a vaccine, of the invention, comprises an RNA molecule in which the nucleic acid sequence of the polynucleotide (or nucleic acid) is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide (or nucleic acid), or mRNA molecule, of the invention, or of a polynucleotide (or nucleic acid), or mRNA molecule, of a pharmaceutical composition, a vector, or a vaccine, of the invention, comprises an mRNA molecule in which the nucleic acid sequence of the polynucleotide (or nucleic acid) is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide (or nucleic acid), or mRNA molecule, of the invention, or of a polynucleotide (or nucleic acid), or mRNA molecule, of a pharmaceutical composition, a vector, or a vaccine, of the invention, comprises an RNA molecule in which the nucleic acid sequence of the polynucleotide (or nucleic acid) is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides.

Optionally a polynucleotide (or nucleic acid) of the invention, or mRNA molecule, or of a polynucleotide (or nucleic acid), or mRNA molecule, of a pharmaceutical composition, a vector, or a vaccine, of the invention, comprises an mRNA molecule in which the nucleic acid sequence of the polynucleotide (or nucleic acid) is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides.

mRNA vaccines of the invention may be co-administered with an immunological adjuvant, for example MF59 (Novartis), TriMix, RNActive (CureVac AG), RNAdjuvant (again reviewed in Wang et al., supra).

There is also provided according to the invention an isolated cell comprising or transfected with a vector of the invention.

There is also provided according to the invention a fusion protein comprising a polypeptide of the invention.

There is further provided according to the invention a pharmaceutical composition comprising an mRNA of the invention, an mRNA vaccine vector of the invention, or an mRNA vaccine of the invention, and a pharmaceutically acceptable carrier, excipient, or diluent.

There is also provided according to the invention an mRNA of the invention, an mRNA vaccine vector of the invention, an mRNA vaccine of the invention, or a pharmaceutical composition of the invention, for use as a medicament.

There is further provided according to the invention an mRNA of the invention, an mRNA vaccine vector of the invention, an mRNA vaccine of the invention, or a pharmaceutical composition of the invention, for use in the prevention, treatment, or amelioration of a coronavirus infection.

There is also provided according to the invention use of an mRNA of the invention, an mRNA vaccine vector of the invention, an mRNA vaccine of the invention, or a pharmaceutical composition of the invention, in the manufacture of a medicament for the prevention, treatment, or amelioration of a coronavirus infection.

There is also provided according to the invention a method of inducing an immune response to a coronavirus in a subject, which comprises administering to the subject an effective amount of an mRNA of the invention, an mRNA vaccine vector of the invention, an mRNA vaccine of the invention, or a pharmaceutical composition of the invention.

There is also provided according to the invention a method of immunising a subject against a coronavirus, which comprises administering to the subject an effective amount of an mRNA of the invention, an mRNA vaccine vector of the invention, an mRNA vaccine of the invention, or a pharmaceutical composition of the invention.

Optionally a method of the invention comprises administering to the subject an mRNA of the invention, an mRNA vaccine vector of the invention, an mRNA vaccine of the invention, or a pharmaceutical composition of the invention, as part of a prime boost regimen.

Optionally the coronavirus is a beta-coronavirus.

Optionally the beta-coronavirus is a lineage B or C beta-coronavirus.

Optionally the beta-coronavirus is a lineage B beta-coronavirus.

Optionally the lineage B beta-coronavirus is SARS-CoV or SARS-CoV-2.

Optionally the lineage C beta-coronavirus is MERS-COV.

Optionally the beta-coronavirus is a variant of concern (VOC).

Optionally the beta-coronavirus is a variant of interest (VOI).

Optionally the beta-coronavirus is a SARS-CoV-2 VOC.

Optionally the beta-coronavirus is a SARS-CoV-2 beta, gamma, delta, or omicron VOC.

Optionally the beta-coronavirus is a SARS-CoV-2 omicron VOC.

Optionally the beta-coronavirus is SARS-CoV-2 omicron BA. 1.

Optionally the beta-coronavirus is a SARS-CoV-2 omicron BA.2.

Optionally the beta-coronavirus is a SARS-CoV-2 omicron BA.2.12

Optionally the beta-coronavirus is a SARS-CoV-2 omicron BA.4.

Optionally the beta-coronavirus is a SARS-CoV-2 omicron BA.5.

Optionally the beta-coronavirus is a SARS-CoV-2 omicron XBB.

Optionally the beta-coronavirus is a SARS-CoV-2 omicron XBB.1.5.

Optionally the beta-coronavirus is a SARS-CoV-2 omicron BA.2.12.1.

Optionally the beta-coronavirus is a SARS-CoV-2 omicron BA.2.75.2.

Optionally the beta-coronavirus is a SARS-CoV-2 omicron BQ. 1.1.

Optionally the beta-coronavirus is a SARS-CoV-2 omicron BA.2.3.20.

Optionally the beta-coronavirus is a SARS-CoV-2 omicron BA.2.75.

Optionally the beta-coronavirus is a SARS-CoV-2 omicron BF.7.

Optionally the beta-coronavirus is a SARS-CoV-2 omicron XBB. 1.19.1.

Optionally the beta-coronavirus is a SARS-CoV-2 omicron XBC.

Optionally the beta-coronavirus is a SARS-CoV-2 omicron BQ. 1.12.

Optionally the beta-coronavirus is a SARS-CoV-2 omicron CH.1.1.1.

Optionally the beta-coronavirus is a SARS-CoV-2 omicron XBB.1.9.1.

Optionally the subject is a human subject.

### Pharmaceutical Compositions

According to the invention there is also provided a pharmaceutical composition comprising an isolated polypeptide of the invention, and a pharmaceutically acceptable carrier, excipient, or diluent.

Optionally a pharmaceutical composition of the invention comprises more than one different isolated polypeptide of the invention.

According to the invention there is also provided a pharmaceutical composition comprising a nucleic acid of the invention, and a pharmaceutically acceptable carrier, excipient, or diluent. Optionally a pharmaceutical composition of the invention comprises more than one nucleic acid molecule of the invention encoding a different polypeptide of the invention. According to the invention there is also provided a pharmaceutical composition comprising a mRNA molecule of the invention, and a pharmaceutically acceptable carrier, excipient, or diluent.

Optionally a pharmaceutical composition of the invention comprises more than one mRNA molecule of the invention encoding a different polypeptide of the invention.

According to the invention there is also provided a pharmaceutical composition comprising a vector of the invention, and a pharmaceutically acceptable carrier, excipient, or diluent. Optionally a pharmaceutical composition of the invention further comprises an adjuvant for enhancing an immune response in a subject to the polypeptide, or to a polypeptide encoded by the nucleic acid, of the composition.

Optionally a pharmaceutical composition of the invention further comprises an adjuvant for enhancing an immune response in a subject to the polypeptides, or to polypeptides encoded by the nucleic acids, of the composition.

There is also provided according to the invention a pseudotyped virus comprising a polypeptide of the invention.

### Methods of Treatment and Uses

There is also provided according to the invention a method of inducing an immune response to a coronavirus in a subject, which comprises administering to the subject an effective amount of a polypeptide of the invention, a nucleic acid of the invention, an mRNA of the invention, a vector of the invention, a pharmaceutical composition, or a vaccine of the invention.

There is also provided according to the invention a method of immunising a subject against a coronavirus, which comprises administering to the subject an effective amount of a polypeptide of the invention, a nucleic acid of the invention, an mRNA of the invention, a vector of the invention, a vaccine of the invention, or a pharmaceutical composition of the invention.

An effective amount is an amount to produce an antigen-specific immune response in a subject.

There is further provided according to the invention a polypeptide of the invention, a nucleic acid of the invention, an mRNA of the invention, a vector of the invention, a vaccine of the invention, or a pharmaceutical composition of the invention, for use as a medicament.

There is further provided according to the invention a polypeptide of the invention, a nucleic acid of the invention, an mRNA of the invention, a vector of the invention, a vaccine of the invention, or a pharmaceutical composition of the invention, for use in the prevention, treatment, or amelioration of a coronavirus infection.

There is also provided according to the invention use of a polypeptide of the invention, a nucleic acid of the invention, an mRNA of the invention, a vector of the invention, a vaccine of the invention, or a pharmaceutical composition of the invention, in the manufacture of a medicament for the prevention, treatment, or amelioration of a coronavirus infection.

Optionally the coronavirus is a β-coronavirus.

Optionally the β-coronavirus is a lineage B or C β-coronavirus.

Optionally the β-coronavirus is a lineage B β-coronavirus.

Optionally the lineage B β-coronavirus is SARS-CoV or SARS-CoV-2.

Optionally the lineage C β-coronavirus is MERS-COV.

Optionally an immune response is induced against more than one lineage B beta-coronavirus.

Optionally an immune response is induced against SARS-1 and SARS-2 beta-coronavirus.

Optionally an immune response is induced against SARS-1 and MERS beta-coronavirus.

Optionally an immune response is induced against SARS-2 and MERS beta-coronavirus.

Optionally an immune response is induced against SARS-1, SARS-2, and MERS beta-coronavirus.

Optionally the beta-coronavirus is a variant of concern (VOC).

Optionally the beta-coronavirus is a SARS-CoV-2 VOC.

Optionally the beta-coronavirus is a SARS-CoV-2 lineage B1.248 (Brazil P1 lineage) VOC.

Optionally the beta-coronavirus is a SARS-CoV-2 lineage B1.351 (South Africa) VOC.

Optionally the beta-coronavirus is a SARS-CoV-2 beta, gamma, or delta VOC.

Optionally the beta-coronavirus is a SARS-CoV-2 beta VOC.

Optionally the beta-coronavirus is a SARS-CoV-2 gamma VOC.

Optionally the beta-coronavirus is a SARS-CoV-2 delta VOC.

Optionally the beta-coronavirus is a SARS-CoV-2 alpha VOC.

Optionally the beta-coronavirus is a SARS-CoV-2 omicron VOC.

Optionally the beta-coronavirus is SARS-CoV-2 omicron BA. 1.

Optionally the beta-coronavirus is a SARS-CoV-2 omicron BA.2.

Optionally the beta-coronavirus is a SARS-CoV-2 omicron BA.2.12. Optionally the beta-coronavirus is a SARS-CoV-2 omicron BA.4.

Optionally the beta-coronavirus is a SARS-CoV-2 omicron BA.5.

Optionally the beta-coronavirus is a SARS-CoV-2 omicron XBB.

Optionally the beta-coronavirus is a SARS-CoV-2 omicron XBB.1.5.

Optionally the beta-coronavirus is a SARS-CoV-2 omicron BA.2.12.1.

Optionally the beta-coronavirus is a SARS-CoV-2 omicron BA.2.75.2.

Optionally the beta-coronavirus is a SARS-CoV-2 omicron BQ.1.1.

Optionally the beta-coronavirus is a SARS-CoV-2 omicron BA.2.3.20.

Optionally the beta-coronavirus is a SARS-CoV-2 omicron BA.2.75.

Optionally the beta-coronavirus is a SARS-CoV-2 omicron BF.7.

Optionally the beta-coronavirus is a SARS-CoV-2 omicron XBB.1.19.1.

Optionally the beta-coronavirus is a SARS-CoV-2 omicron XBC.

Optionally the beta-coronavirus is a SARS-CoV-2 omicron BQ. 1.12.

Optionally the beta-coronavirus is a SARS-CoV-2 omicron CH. 1.1.1.

Optionally the beta-coronavirus is a SARS-CoV-2 omicron XBB. 1.9.1.

It can readily be determined whether an immune response has been induced to a beta-coronavirus using methods well-known to the skilled person. For example, a pseudotype neutralisation assay as described in any of the examples below may be used.

### Administration

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration routes but also intradermal, transdermal, subcutaneous, or intramuscular injections or intralesional, intracranial, intrapulmonal, intracardial, and sublingual injections.

For lipid nanoparticles, the administration route is often determined by the properties of the nanoparticles and therapeutic indications. After intravenous (i.v.) administration, many lipid nanoparticles can accumulate in the liver. The liver is inherently capable of producing secretory proteins and, therefore, i.v. administration of lipid nanoparticle-mRNA formulations can be used to produce proteins that are missing in inherited metabolic and haematological disorders, or to produce antibodies to neutralize pathogens or target cancer cells. These applications require protein translation without stimulation of an immune response, which may limit the efficiency of repeated dosing. However, i.v. administration may also lead to accumulation of lipid nanoparticles in multiple lymph nodes throughout the body, which could increase immune responses to mRNA vaccines. For example, i.v. administration of mRNA vaccines has been shown to induce stronger antigen-specific cytotoxic T cell responses compared with local injection. Broad distribution of mRNA vaccines in the body may lead to systemic adverse effects, and, thus, it may be necessary to develop lipid nanoparticles that allow targeted delivery of mRNA vaccines into tissues with abundant immune cells.

Topical administration routes have also been explored for mRNA therapeutics. Topical administration aims at achieving local therapeutic effects; for example, local injection of lipid nanoparticle-mRNA formulations enables supplementation of therapeutic proteins in specific tissues, such as heart, eyes and brain. Moreover, lipid nanoparticle-mRNA formulations can be administered into the lungs by inhalation.

Local administration of mRNA vaccines can also prime systemic responses; for example, intradermal (i.d.), intramuscular (i.m.) and subcutaneous (s.c.) injection are commonly used for vaccination, because resident and recruited antigen-presenting cells (APCs) are present in the skin and muscle, which can internalize and process mRNA-encoded antigens. Furthermore, the vascular and lymphatic vessels of these tissues help APCs and mRNA vaccines to centre the draining lymph nodes to stimulate T cell immunity. Indeed, both i.m.

and i.d. administration of lipid nanoparticle-mRNA vaccines produce robust immune responses at a well-tolerated dose in human trials. Vaccination can also be done by intranasal administration, because APCs in the peripheral lymph nodes can readily endocytose administered lipid nanoparticle-mRNA formulations.

mRNA vaccines delivered by lipid nanoparticle may comprise cationic lipids and/or ionisable lipids, see review: Lipid Nanoparticles for mRNA Delivery, Nature Reviews Materials, 6 1078-1094, 2021. In addition to cationic or ionizable lipids, lipid nanoparticle-mRNA formulations typically contain other lipid components, such as phospholipids (for example, phosphatidylcholine and phosphatidylethanolamine), cholesterol or polyethylene glycol (PEG)-functionalized lipids (PEG-lipids). These lipids can improve nanoparticle properties, such as particle stability, delivery efficacy, tolerability and biodistribution. Compositions of the invention may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

The present disclosure includes methods comprising administering an mRNA vaccine, or a DNA vaccine to a subject in need thereof. The exact amount required will vary from subject to subject, depending on the species, age, and general condition of the subject, the severity of the disease, the particular composition, its mode of administration, its mode of activity, and the like.

The mRNA or DNA is typically formulated in dosage unit form for ease of administration and uniformity of dosage. It will be understood, however, that the total daily usage of the mRNA or DNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the mRNA or DNA, as provided herein, may be as low as 20 μg, administered for example as a single dose or as two 10 μg doses. In some embodiments, the effective amount is a total dose of 20 μg-300 μg or 25 μg-300 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 ug, 160 μg, 170 μg, 180 μg, 190 μg, 200 μg, 250 μg, or 300 μg. In some embodiments, the effective amount is a total dose of 20 μg. In some embodiments, the effective amount is a total dose of 25 μg. In some embodiments, the effective amount is a total dose of 50 μg. In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg.

The mRNA or DNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

Optionally, an mRNA or DNA vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

Optionally a dosage of between 10 μg/kg and 400 μg/kg of the nucleic acid vaccine is administered to the subject. In some embodiments the dosage of the mRNA or DNA polynucleotide (or nucleic acid) is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

In some embodiments, the mRNA vaccine is administered to the subject by intradermal or intramuscular injection. In some embodiments, the mRNA vaccine is administered to the subject on day zero. In some embodiments, a second dose of the mRNA vaccine is administered to the subject on day twenty one.

In a strategy called “prime-boost”, a first dose of the mRNA vaccine is given as a priming step, followed by a second dose as a booster. The prime-boost strategy aims to provide a stronger overall immune response. The boost may be administered at least a day, at least a week, or at least two, three, four, five, six, or seven weeks, or at least two, three, four, five, or six months after the primer. For example, the boost may be administered at least three weeks after the primer.

### Pharmaceutically Acceptable Carriers

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides).

The pharmaceutically acceptable carriers (vehicles) useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of one or more therapeutic compositions, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a polypeptide, nucleic acid, composition, or an mRNA vaccine of the invention is administered intramuscularly.

Optionally a polypeptide, nucleic acid, composition, or an mRNA vaccine of the invention is administered by inhalation.

Optionally a polypeptide, nucleic acid, composition, or an mRNA vaccine of the invention is administered intramuscularly, intradermally, subcutaneously by needle or by gene gun, or electroporation.

### Sequence Identity

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4:29; program available from http://bitincka.com/ledion/matgat), Gap (Needleman & Wunsch, 1970, J. Mol. Biol. 48:443-453), FASTA (Altschul et al., 1990, J. Mol. Biol. 215:403-410; program available from http://www.ebi.ac.uk/fasta), Clustal W 2.0 and X 2.0 (Larkin et al., 2007, Bioinformatics 23:2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

### Conservative Amino Acid Substitutions

A polypeptide encoded by a mRNA of the invention may include one or more conservative amino acid substitutions. Conservative amino acid substitutions are those substitutions that, when made, least interfere with the properties of the original polypeptide, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile: Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

### Broadly Neutralising Immune Response

The term “broadly neutralising immune response” is used herein to mean an immune response elicited in a subject that is sufficient to inhibit (i.e. reduce), neutralise or prevent infection, and/or progress of infection, of a virus within the coronavirus family. Optionally a broadly neutralising immune response is sufficient to inhibit, neutralise or prevent infection, and/or progress of infection, of more than one type of β-coronavirus (for example, SARS-CoV, and SARS-CoV-2). Optionally a broadly neutralising immune response is sufficient to inhibit, neutralise or prevent infection, and/or progress of infection, of more than one type of β-coronavirus within the same β-coronavirus lineage (for example, more than one type of β-coronavirus within the subgenus Sarbecovirus, such as SARS-CoV, SARS-CoV-2, and Bat SL-CoV-WIV1). Optionally a broadly neutralising immune response is sufficient to inhibit, neutralise or prevent infection, and/or progress of infection, of coronaviruses of different β-coronavirus lineages, such as lineage B (for example, SARS-CoV, and SARS-CoV-2) and lineage C (for example, MERS-COV). Optionally a broadly neutralising immune response is sufficient to inhibit, neutralise or prevent infection, and/or progress of infection, of most or all different β-coronaviruses. Optionally a broadly neutralising immune response is sufficient to inhibit, neutralise or prevent infection, and/or progress of infection, of most or all different viruses of the coronavirus family. Optionally a broadly neutralising immune response is sufficient to inhibit, neutralise or prevent infection, and/or progress of infection, of most or all variants of concern (VOCs) of SARS-CoV-2, including Beta, Gamma, Delta, Omicron (BA. 1). Optionally a broadly neutralising immune response is sufficient to inhibit, neutralise or prevent infection, and/or progress of infection, of SARS-CoV, WIV16, RaTG13, SARS-CoV-2, SARS-CoV-2 Beta, SARS-CoV-2 Gamma, SARS-CoV-2 Delta, SARS-CoV-2 Omicron (BA.1, BA. 2, BA.2.12.1, BA.4, BA.5, XBB 1.5). Optionally, a broadly neutralising immune response is sufficient to inhibit, neutralise or prevent infection, and/or progress of infection, of SARS-CoV-2 Omicron BA.2.75, BA.2.75.2, BA.2.3.20, BQ.1.1, BA.2.12, BA.4/5, and XBB. Optionally, a broadly neutralising immune response is sufficient to inhibit, neutralise or prevent infection, and/or progress of infection, of SARS-CoV-2 Omicron BA.2.75, and BA.2.3.20.

The immune response may be a humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide encoded by an mRNA of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

Optionally a polypeptide of the invention is a glycosylated polypeptide.

Optionally a polypeptide encoded by an mRNA of the invention is able to induce the production of antibodies and/or a T-cell response in a human or non-human animal to which the mRNA has been administered (for example, expressed from an administered mRNA vaccine).

Embodiments of the invention are described below, by way of example only, with reference to the accompanying drawings in which:

FIG. 1 shows SARS S-protein architecture. The N-terminal sequence is responsible for relaying extracellular signals intracellularly. Studies show that the N-terminal region of the S protein is much more diverse than the C-terminal region, which is highly conserved (Dong et al, Genomic and protein structure modelling analysis depicts the origin and infectivity of 2019-nCoV, a new coronavirus which caused a pneumonia outbreak in Wuhan, China. 2020). The figure shows the S domain, which comprises S1 and S2 domains, responsible for receptor binding and cell membrane fusion respectively;

FIG. 2 shows details of the greatest VOI in circulation globally as of 3 Apr. 2023. In the table: x=including its sub-lineages (BN, CH and others). Omicron-Omicron Recombinants XBF and XBK that share the same spike as BA.2.75 are monitored under BA.2.75 lineages.

Y=W152R, F157L, 1210V, G257S, D339H, G446S, N460K, Q493 (reversion)

Z=XBB and sub-lineages, excluding XBB.1.5-like lineages. Recombinant lineage of BJ.1 (BA.2.10.1.1) and BM.1.1.1 (BA.2.75.3.1.1.1)

A=Monitoring an umbrella of SARS-CoV-2 lineages that have similar Spike protein profiles and characterised by a specific set of mutations (S: Q183E, S: F486P and S: F490S). This umbrella includes, for instance, the lineages XBB.1.5, XBB. 1.9.1*, XBB.1.9.2*, and XBB.1.16;

FIG. 3 shows alignment of the amino acid sequence of CoV_S_T2_35, CoV_S_T2_36, and Omicron_vaccine with reference sequences SEQ ID NOs: 4-29. FIG. 3a shows alignment of CoV_S_T2_35 at positions wherein the designed sequence comprises novel amino acid residues, as compared with DeltaC. FIG. 3b shows alignment of CoV_S_T2_36 at positions wherein the residue comprises novel amino acid residues, as compared with BA. 1C;

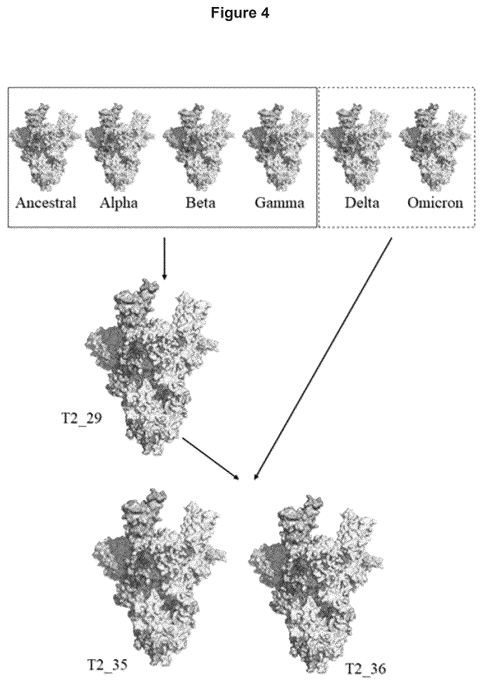

FIG. 4 shows surface representation of the extra-virion region of the spike protein of SARS-CoV-2. The three subunits are coloured in pale yellow (middle subunit), pale blue (left-hand subunit), and grey (right-hand subunit). The mutations reported in different variants are coloured as red (shown as shaded dots in the representations of the variants). The mutations introduced in the spike vaccine antigens are coloured as orange in T2_29, T2_35, and T2_36 (shown as shaded dots in the representations of the designed antigens);

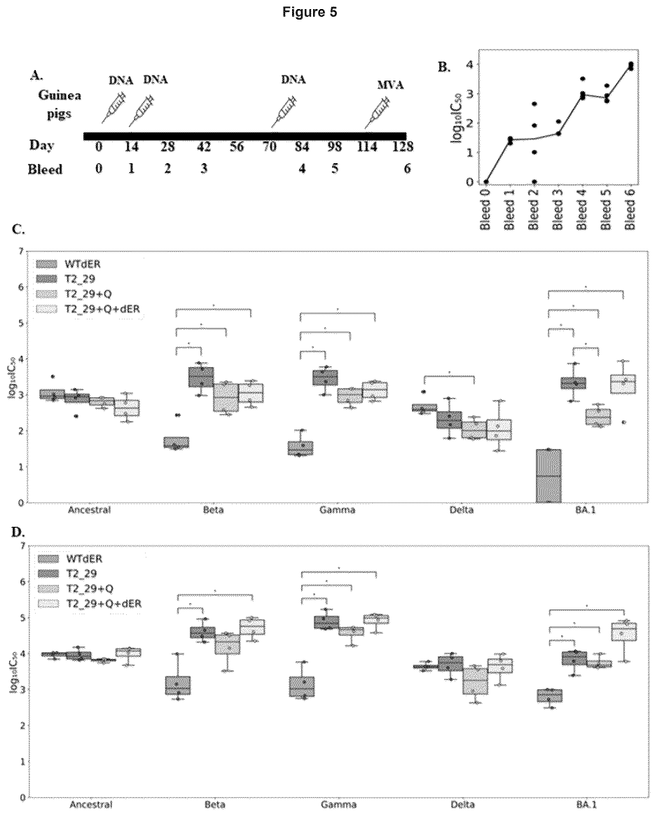

FIG. 5 shows immunisation and bleed schedule (A), with neutralisation data for each bleed (B), and neutralisation data for guinea pigs primed with WTdER, and T2_29, T2_29+Q, and T2_29+Q+dER designed DNA sequences (C), and boosted with T2_29+Q+dER designed MVA sequence (D). The boxplots are colour coded according to primer vaccine, and are in the following order from left-right for each challenge PV: WTdER, and T2_29, T2_29+Q, and T2_29+Q+dER;

FIG. 6 shows neutralisation data for mice primed (B) and boosted (C) with WTdER, Ancestral, and T2_35 and T2_36 mRNA designed sequence. The boxplots are colour coded according to vaccine used, and are in the following order from left-right for each challenge PV: PBS, T2_35, T2_36, BA. 1, Ancestral;

FIG. 7 shows terminal bleed neutralisation data in mice against coronavirus challenge. The boxplots are colour coded according to mRNA vaccine used, and are in the following order from left-right for each challenge PV: PBS, T2_35, T2_36, BA. 1, Ancestral;

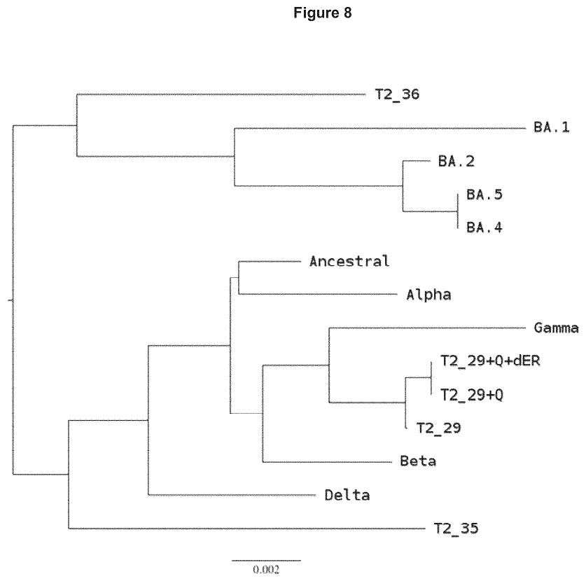

FIG. 8 shows a distance based phylogenetic tree for wild type coronavirus viruses and designed sequences, generated using observed distance;

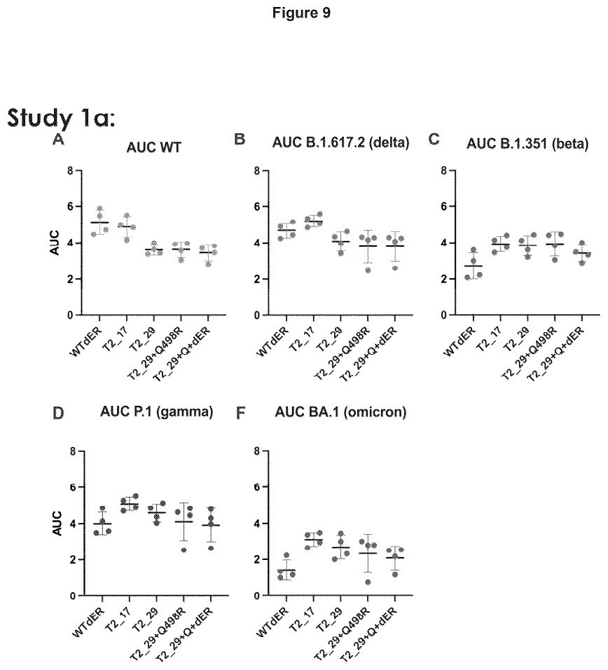

FIG. 9 shows VOC RBD binding antibody levels of guinea pigs at bleed 4 of the schedule of FIG. 5a, shown by ELISA;

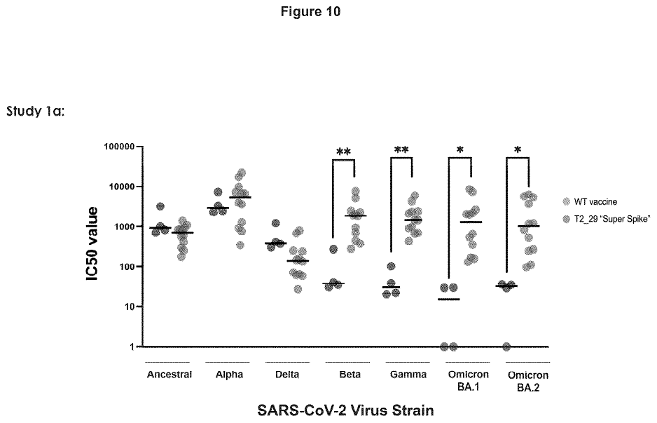

FIG. 10 shows neutralisation titre of guinea pigs immunised with DNA T2_29 vaccine groups. The data points for the WT vaccine appear on the left for each coronavirus pseudovirus, and the data points for the combined T2_29 vaccine appear on the right for each coronavirus pseudotype;

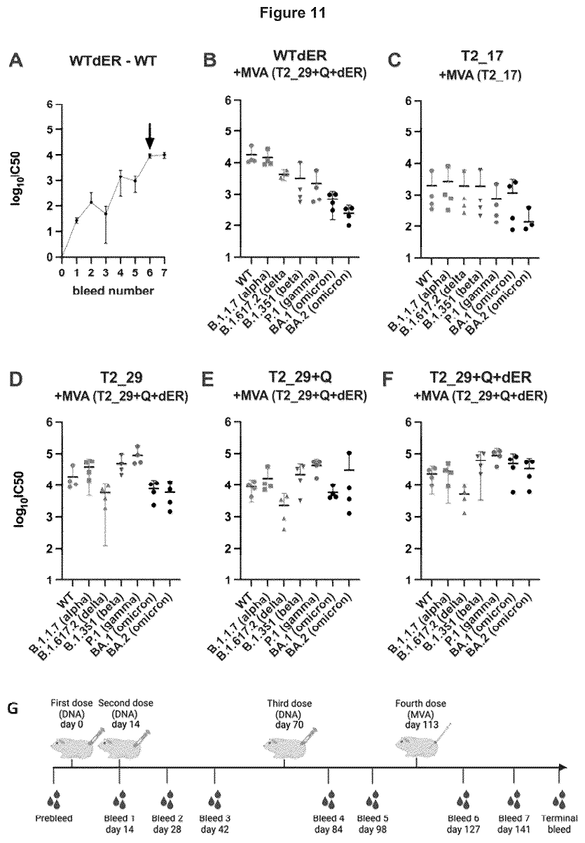

FIG. 11 shows neutralisation data for guinea pigs immunised with designed T2_29 DNA constructs and then boosted with MVA T2_29+Q+dER;

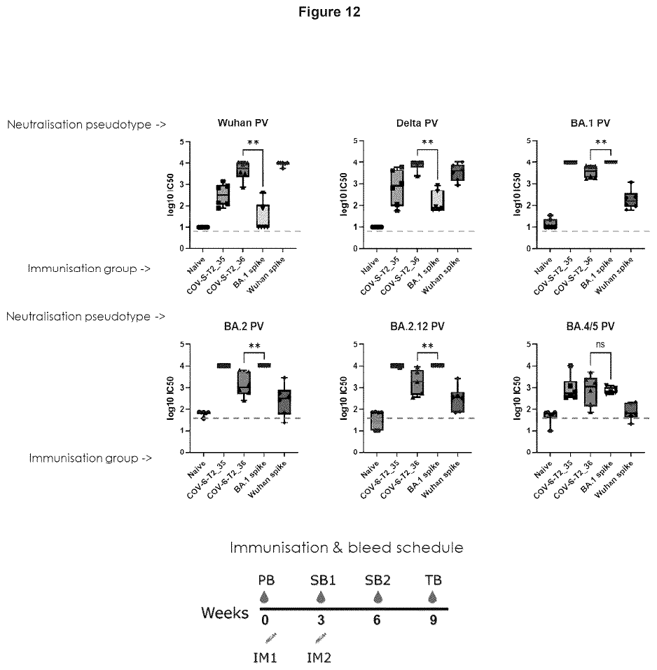

FIG. 12 shows neutralisation data for mice immunised with T2_35 and T2_36 mRNA vaccines, showing statistical comparisons between COV-S-T2_36 vs BA. 1;

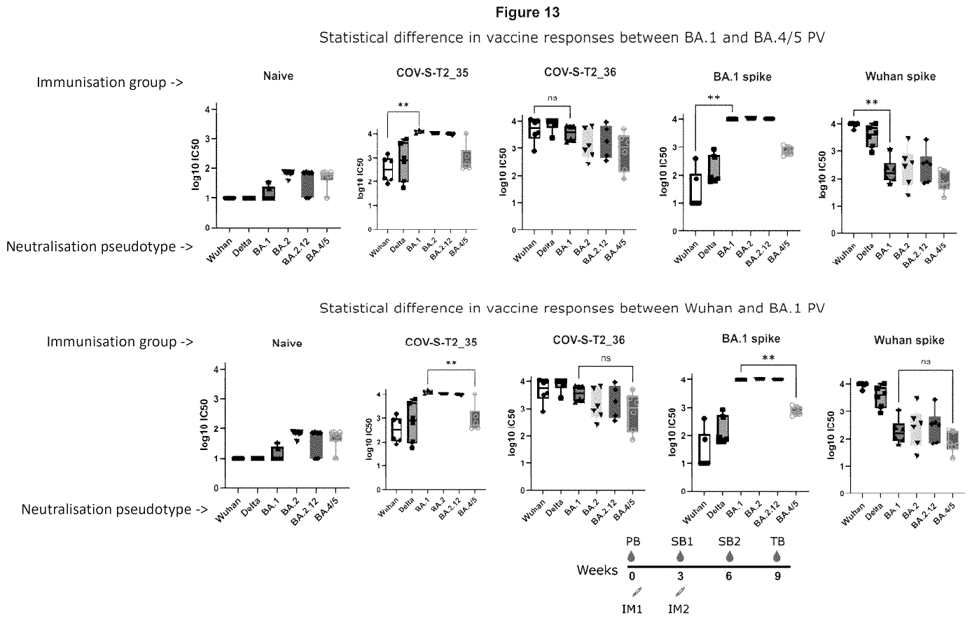

FIG. 13 shows neutralisation data for mice immunised with T2_35 and T2_36 mRNA vaccines, showing statistical comparisons, all vaccine groups IC50 neutralisation values;

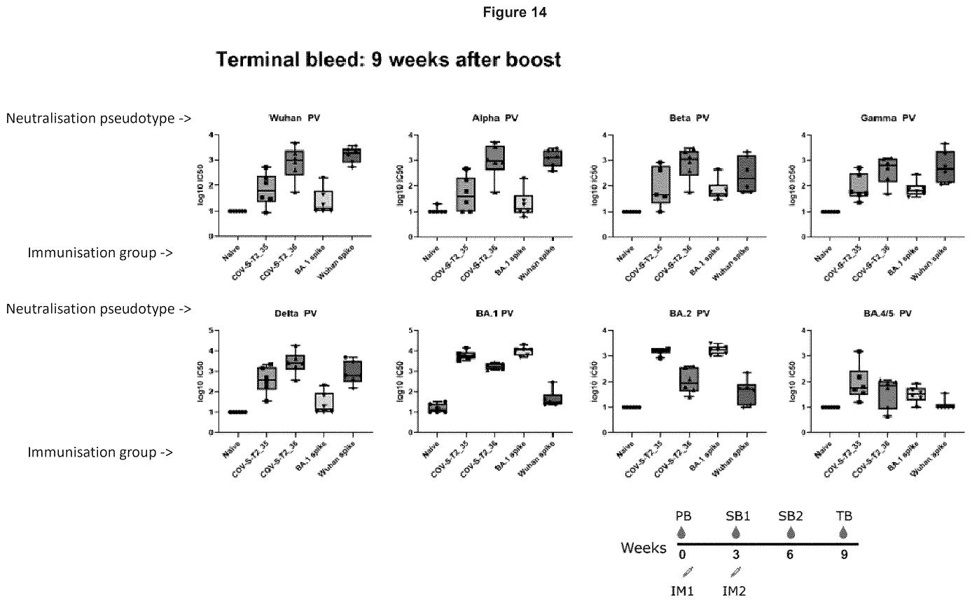

FIG. 14 shows neutralisation data for mice immunised with T2_35 and T2_36 mRNA vaccines, showing statistical comparisons, all vaccine groups IC50 neutralisation values;

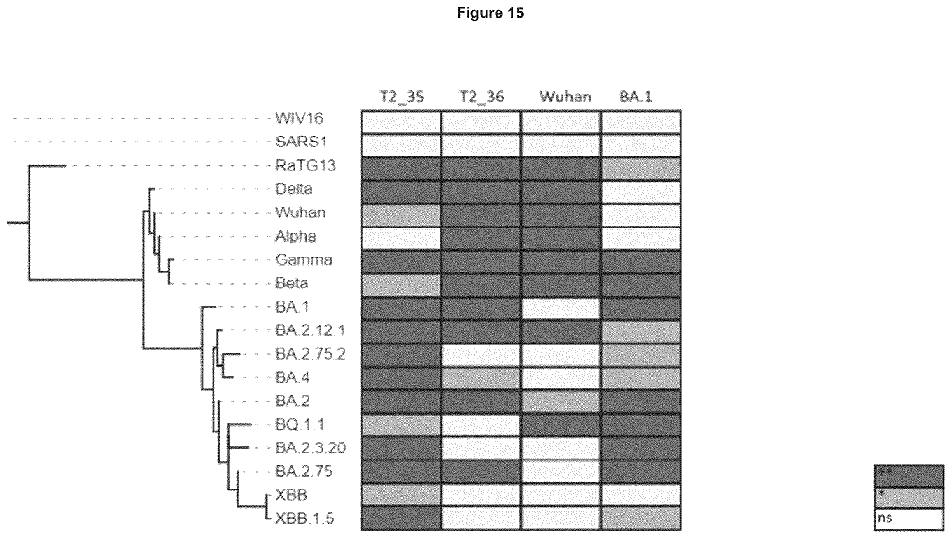

FIG. 15 shows neutralisation data for mice immunised with T2_35 and T2_36 mRNA vaccines, and Wuhan and BA.1 Spike vaccines, against SARS-CoV-1 and SARS-like pseudoviruses. Data based on statistical difference between immunised and naïve animals. Mann Whitney U-test (white=ns, light grey=* p<0.05, dark grey=** p<0.01);

FIG. 16 shows an amino acid sequence alignment of CoV_S_T2_35(deom) (SEQ ID NO:1) with CoV_S_T3_1(Deom_v2) (SEQ ID NO:31), and CoV_S_T3_2(Deom_v3) (SEQ ID NO: 32). Differences between the sequences are shown as the boxed residues;

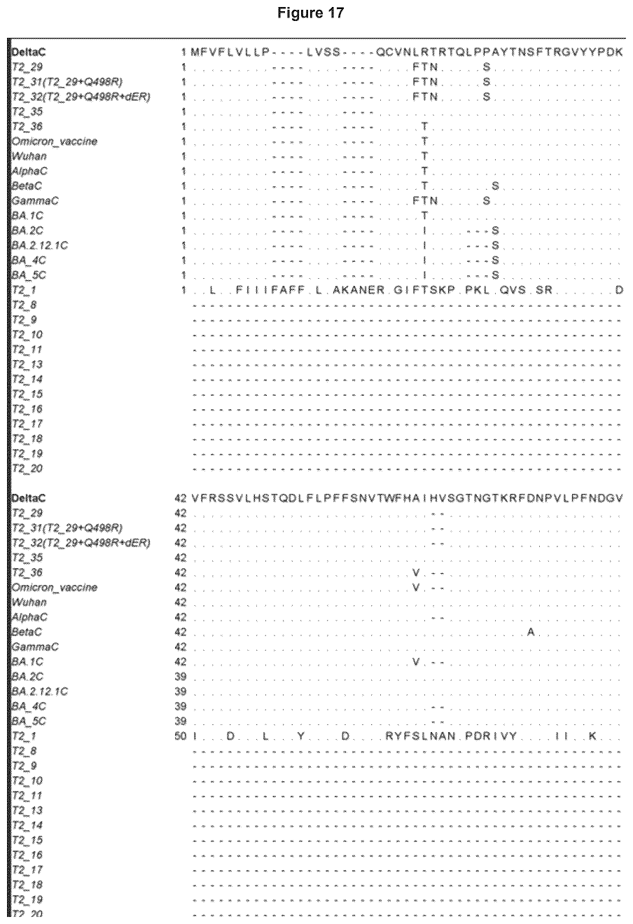

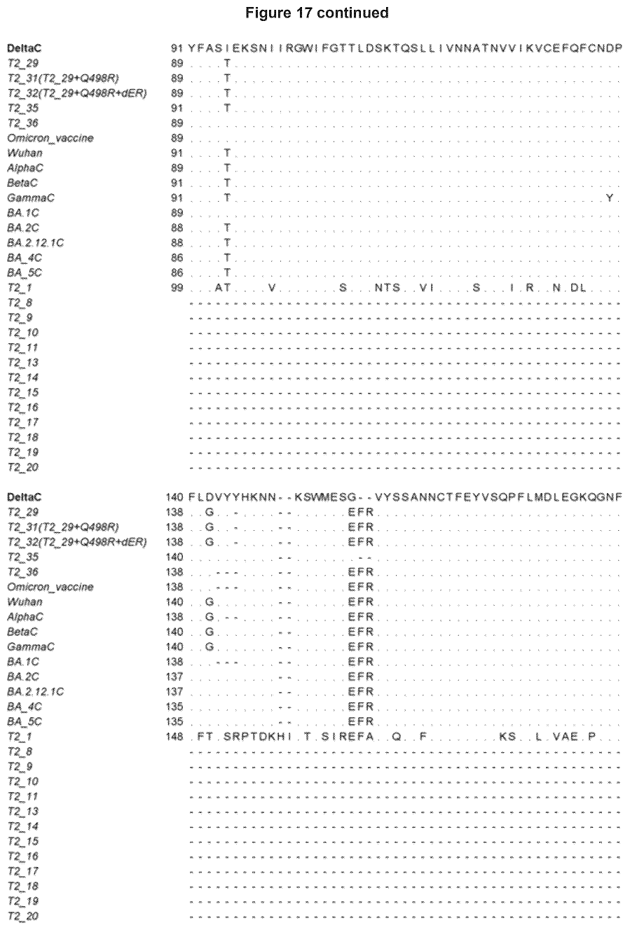

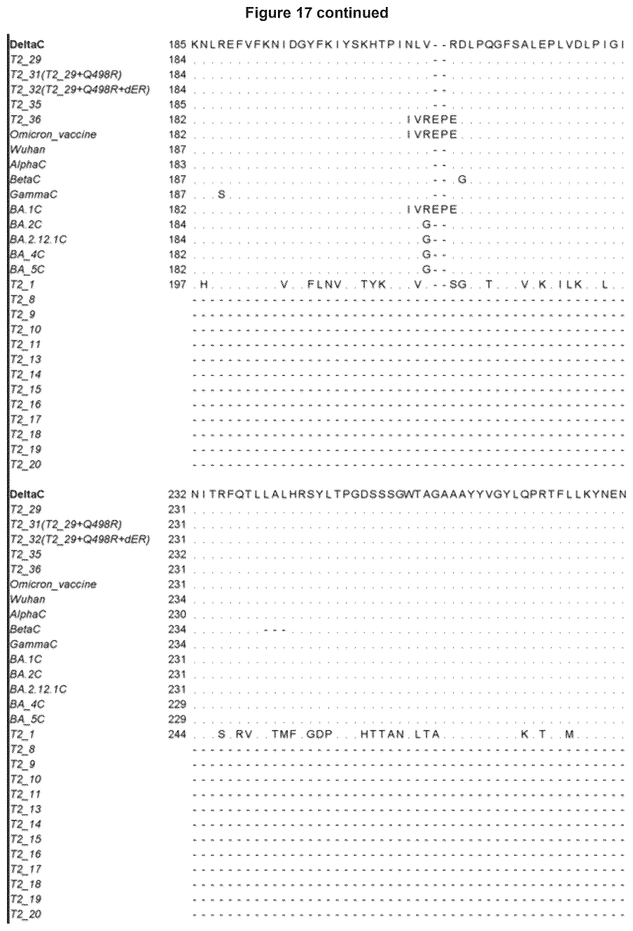

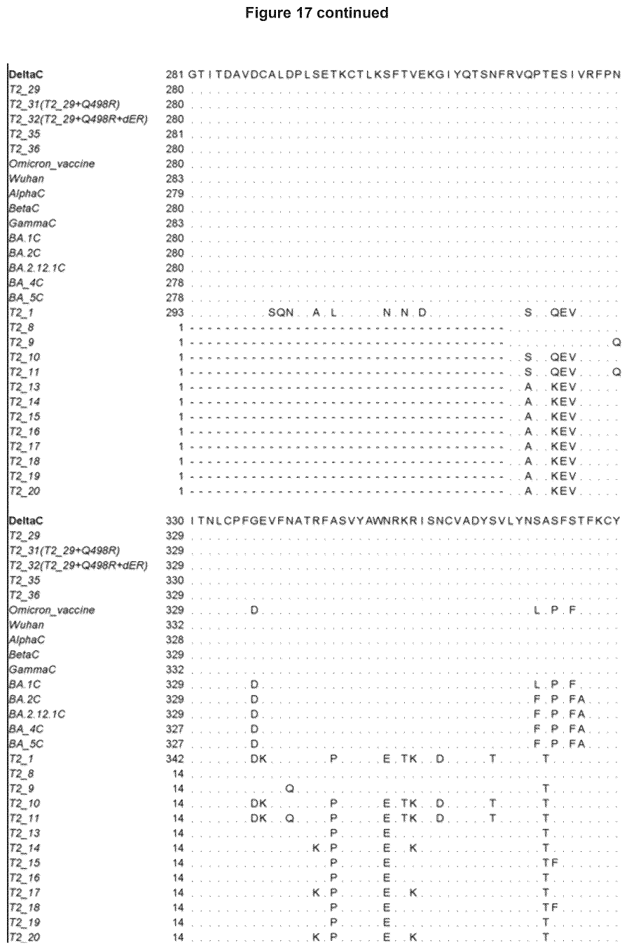

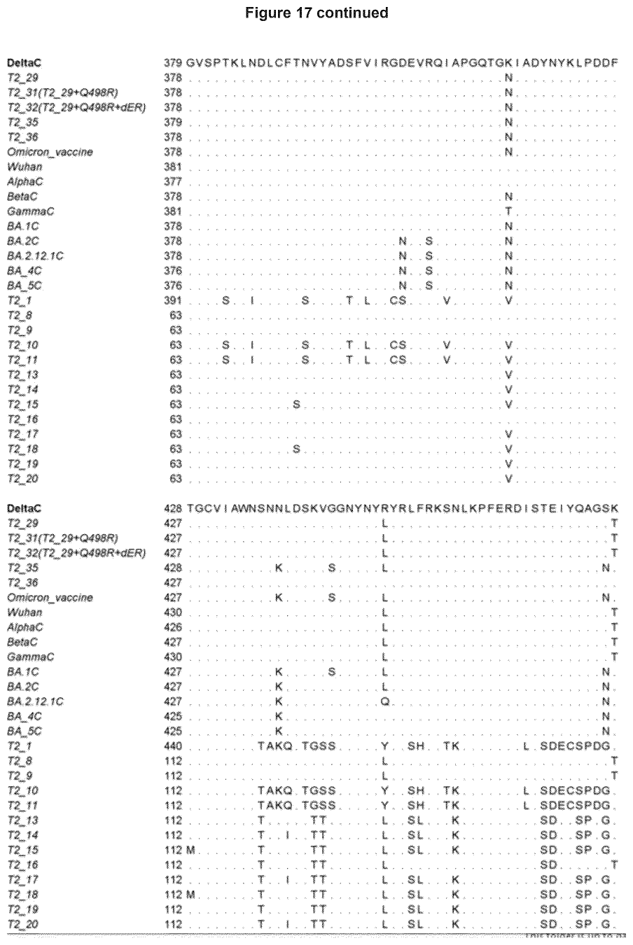

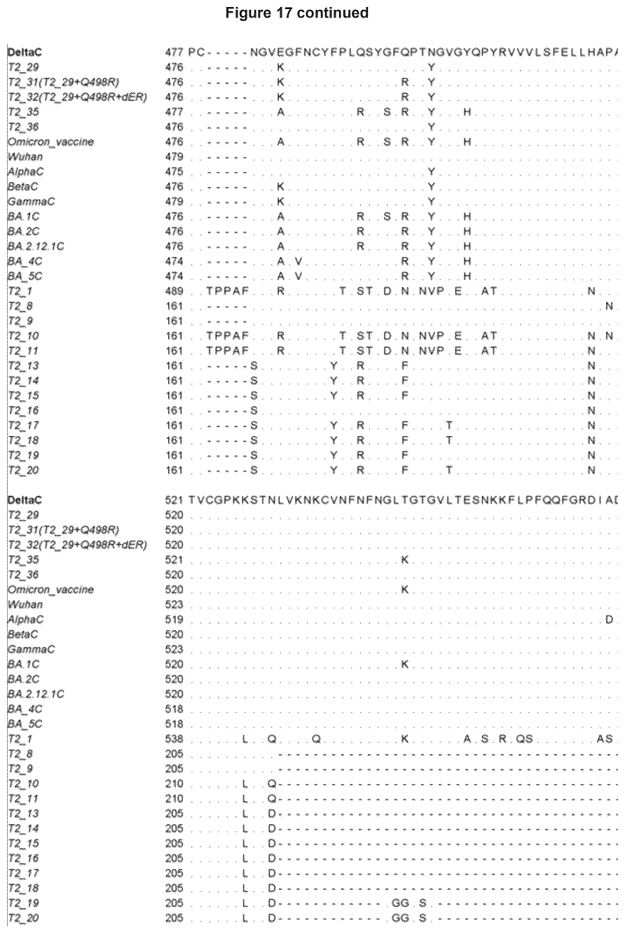

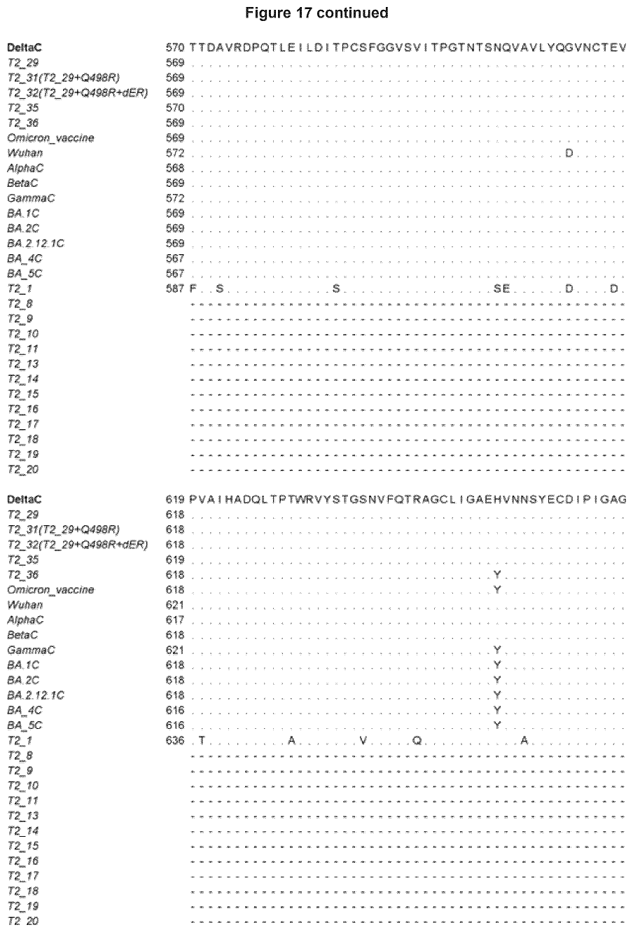

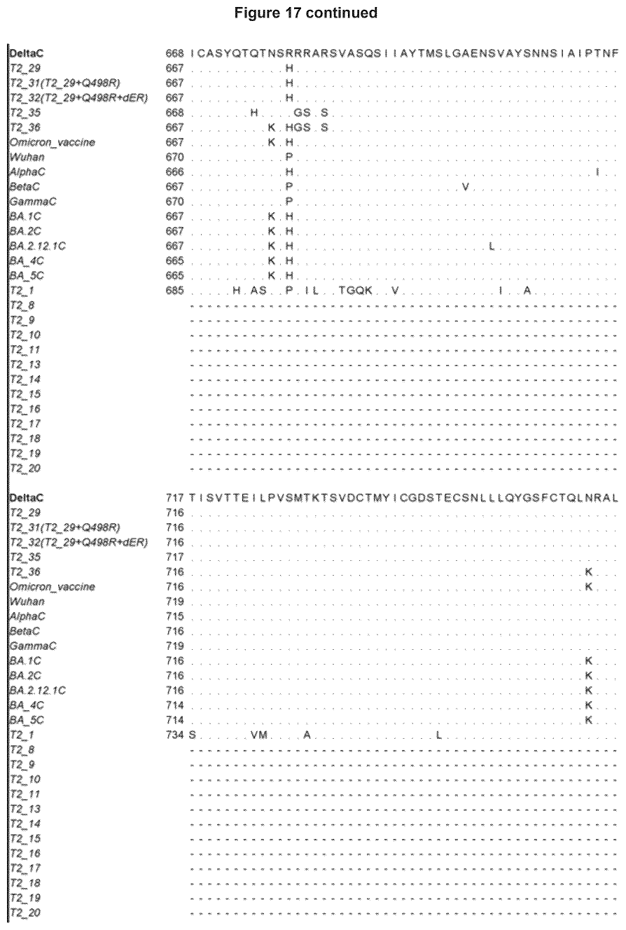

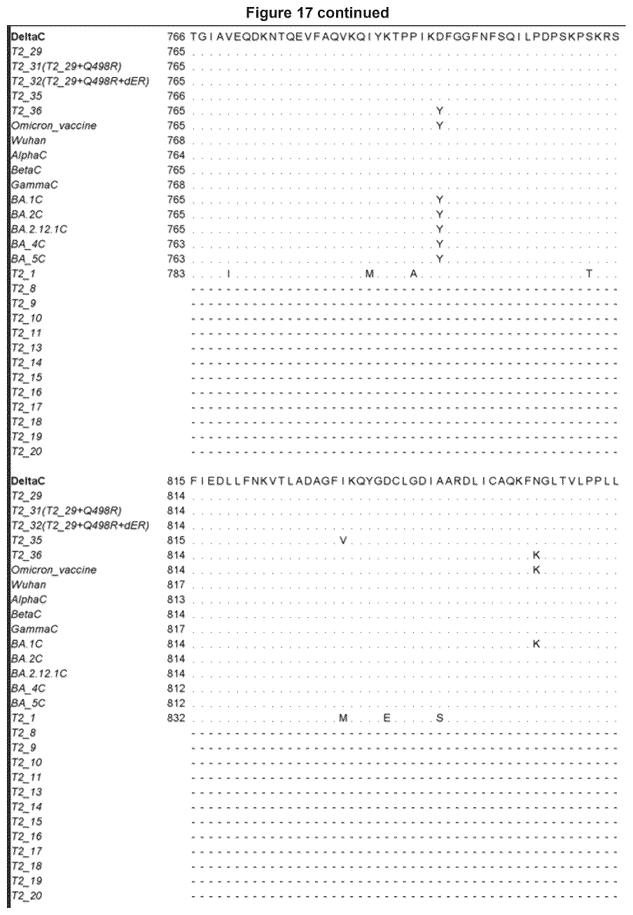

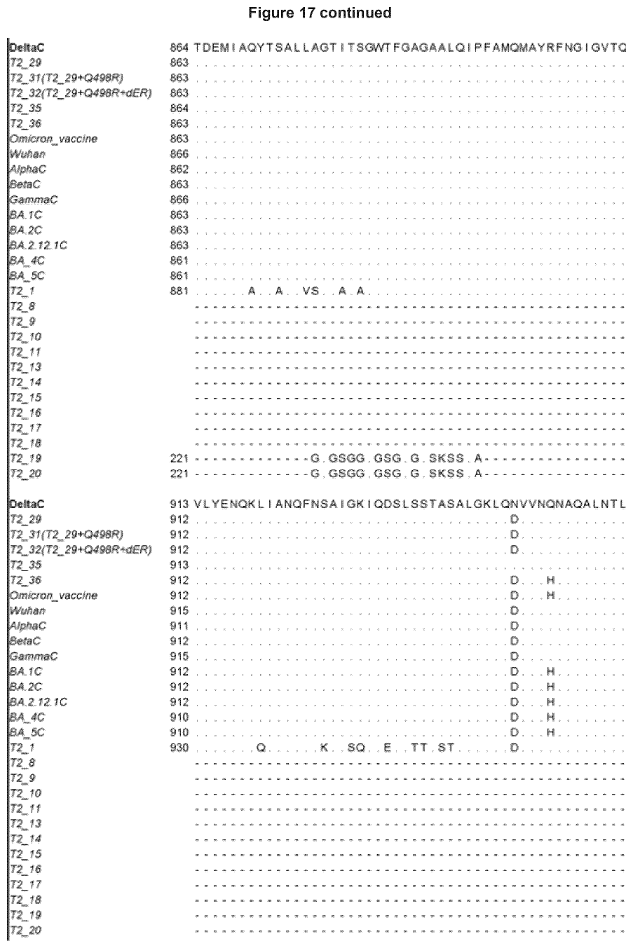

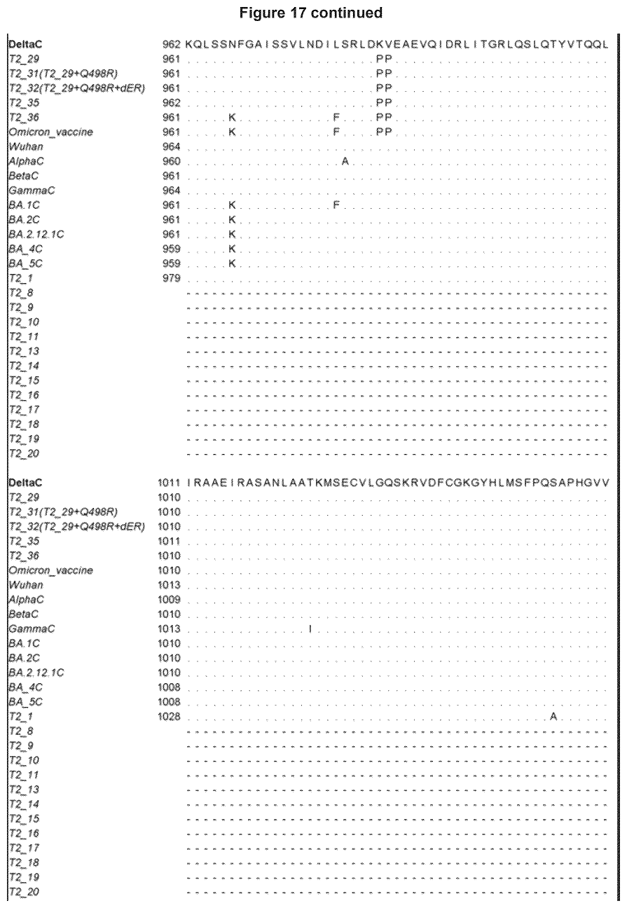

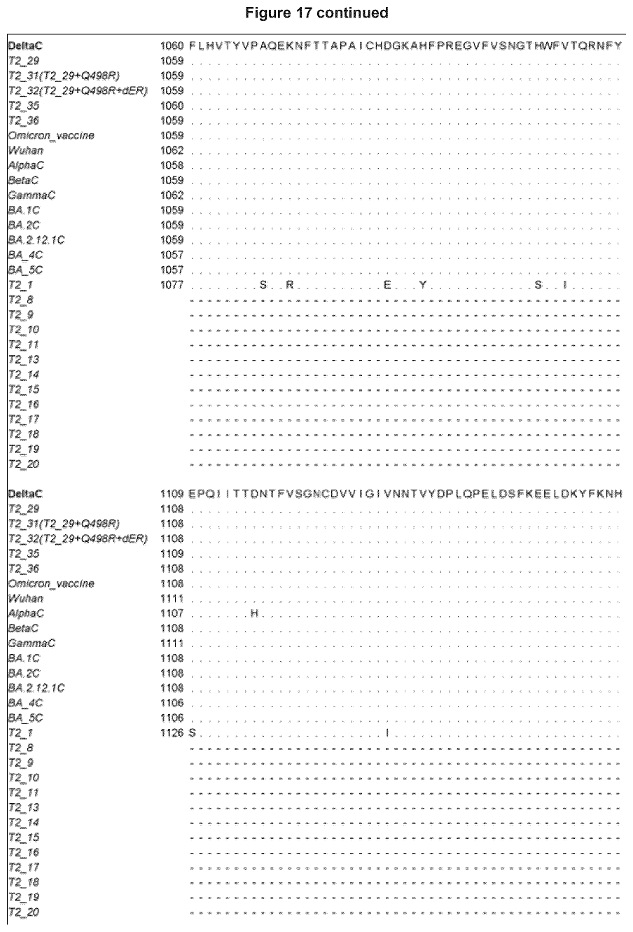

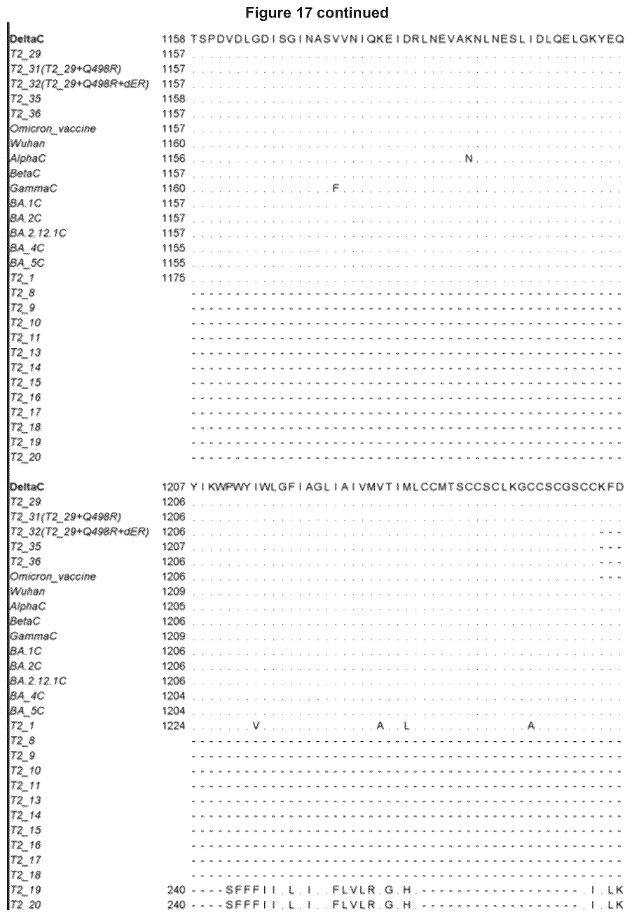

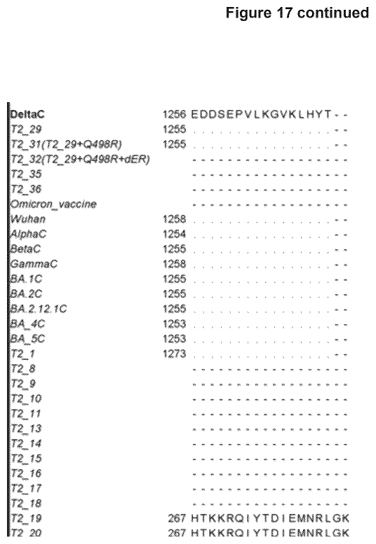

FIG. 17 shows an amino acid sequence alignment of designed S proteins CoV_S_T2_35(deom) (SEQ ID NO:1) and CoV_S_T2_36 (omide) (SEQ ID NO:2) with previously designed S protein sequence, RBD sequence, and reference sequences, with DeltaC as the alignment reference sequence;

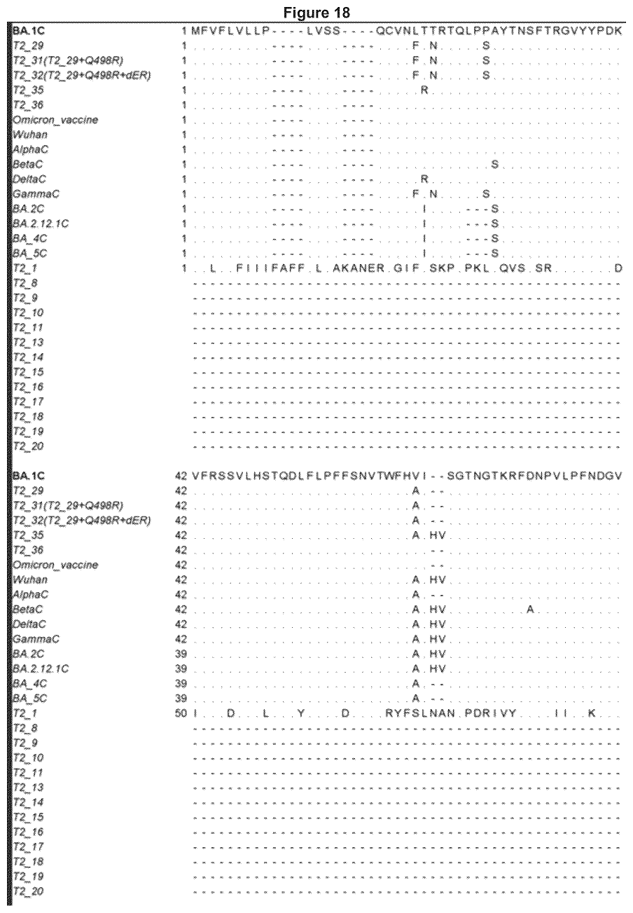

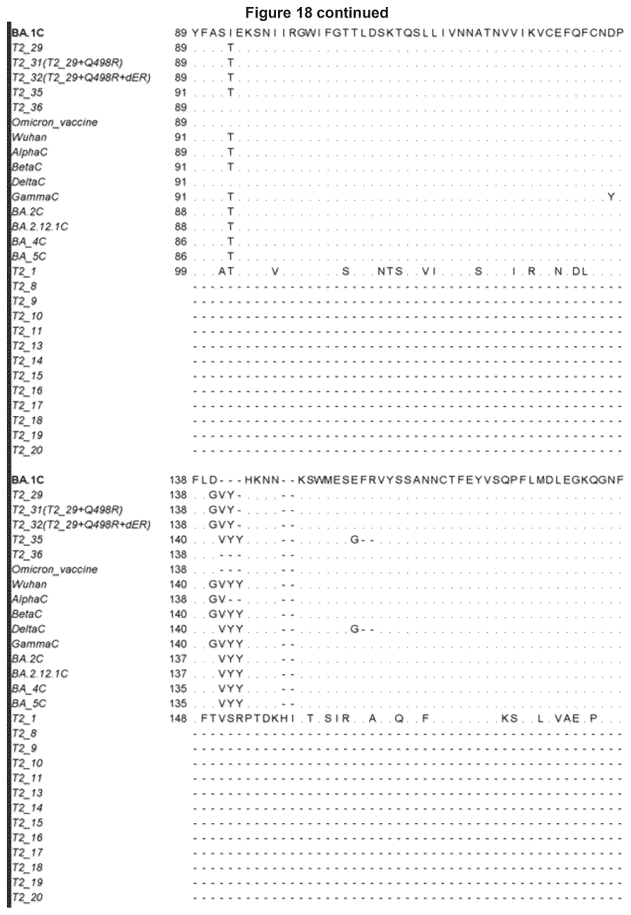

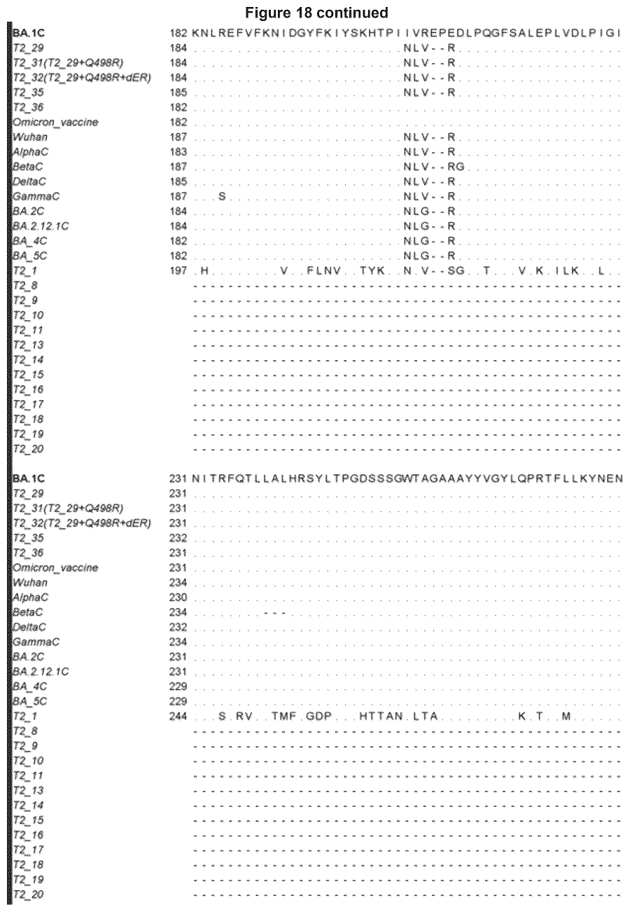

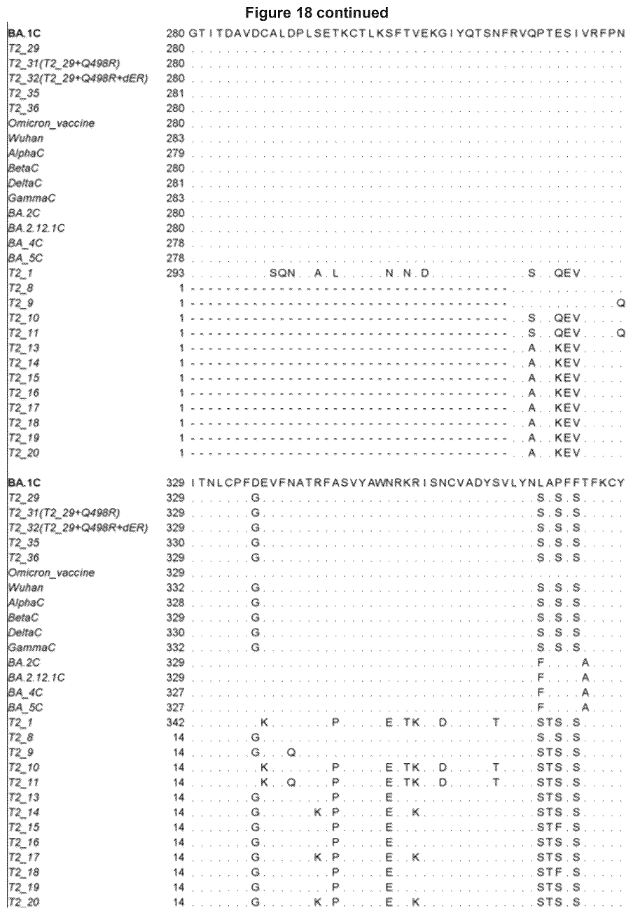

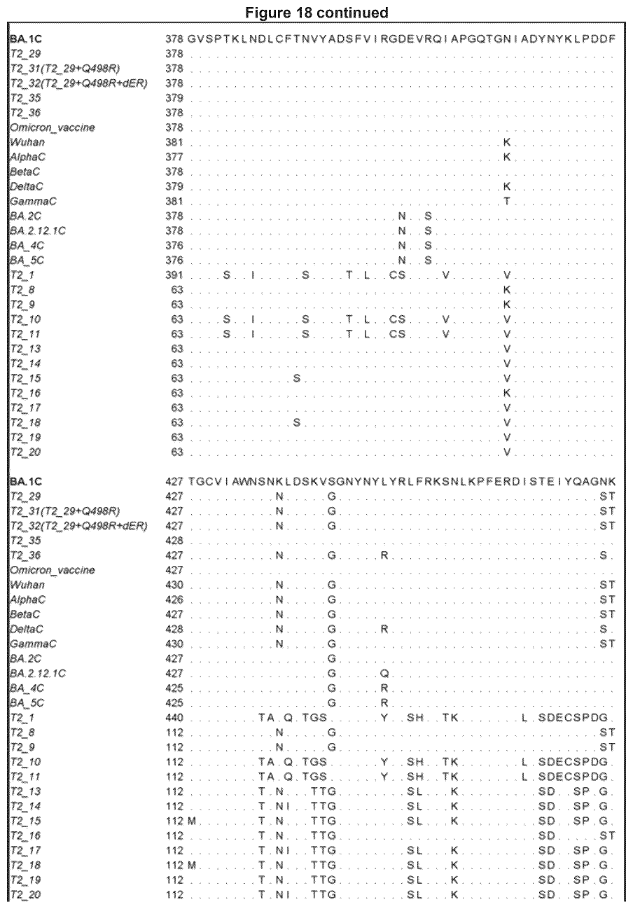

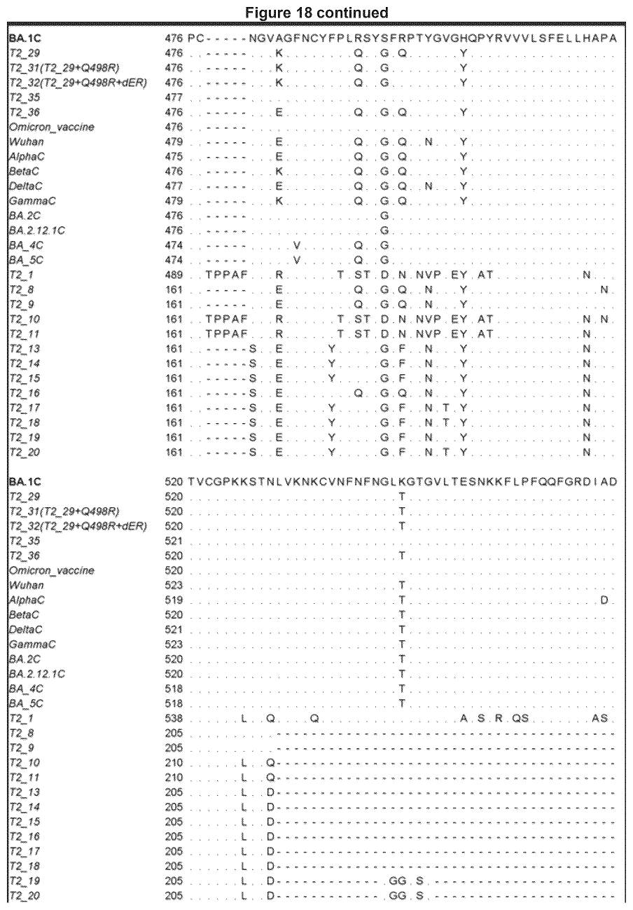

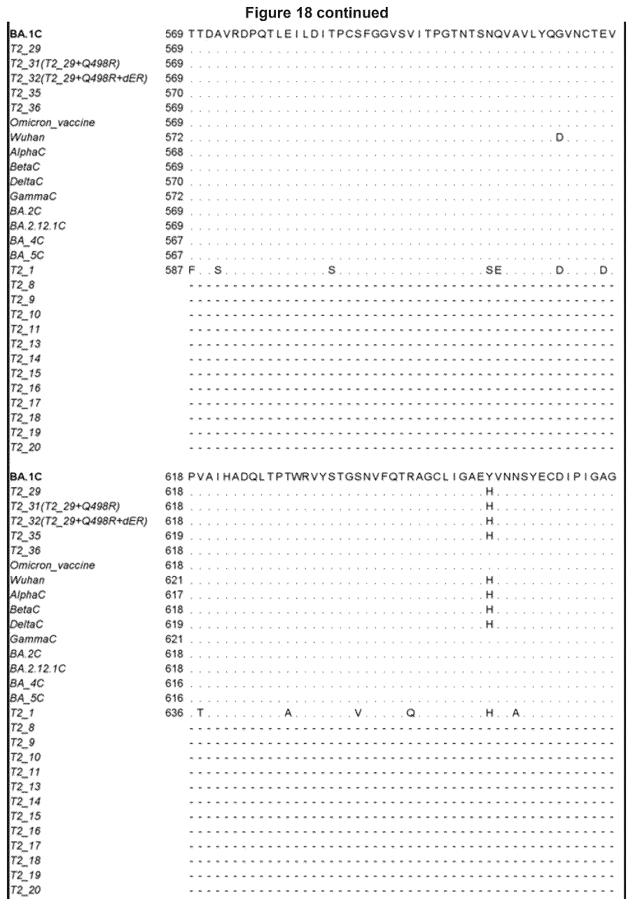

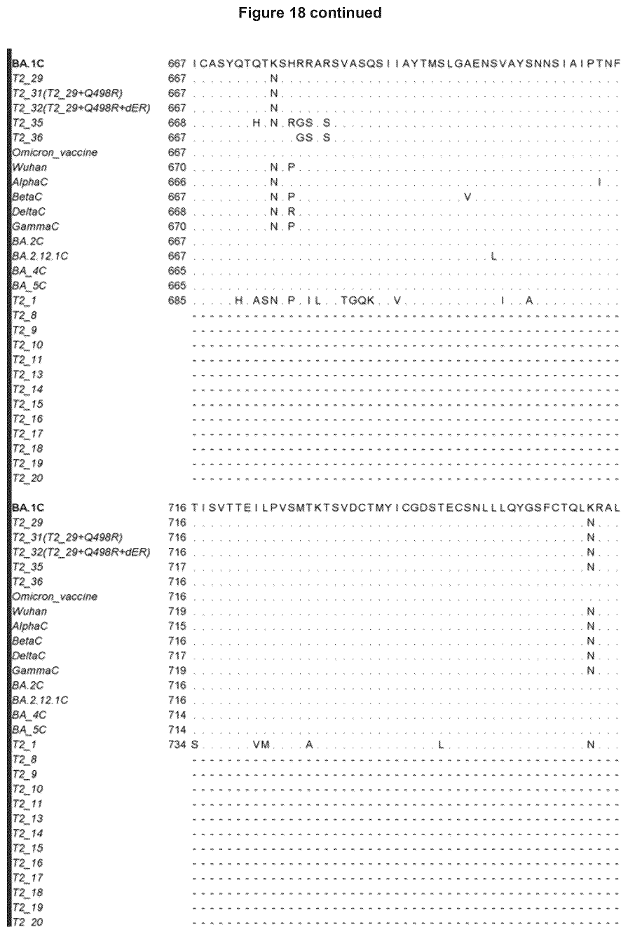

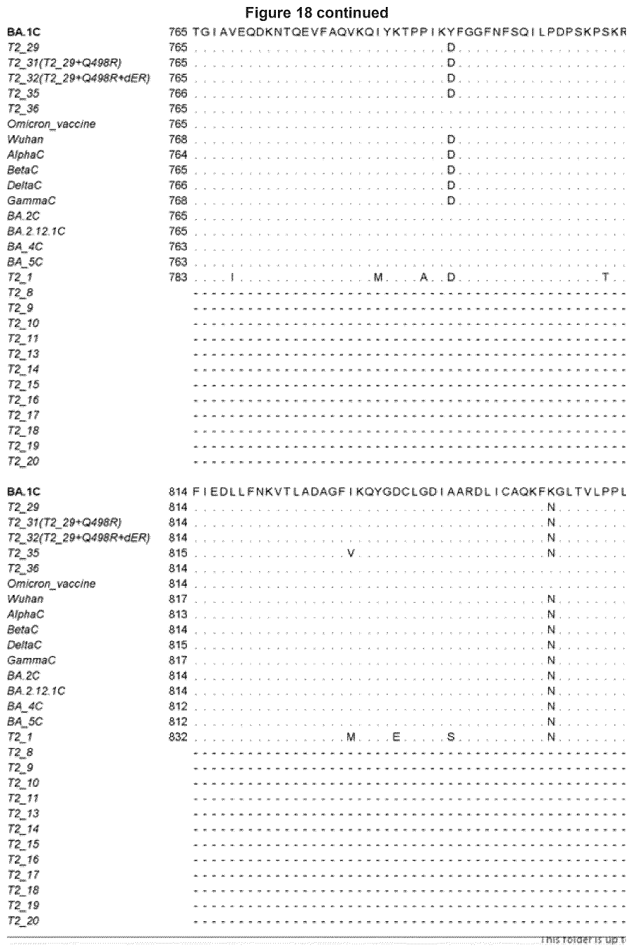

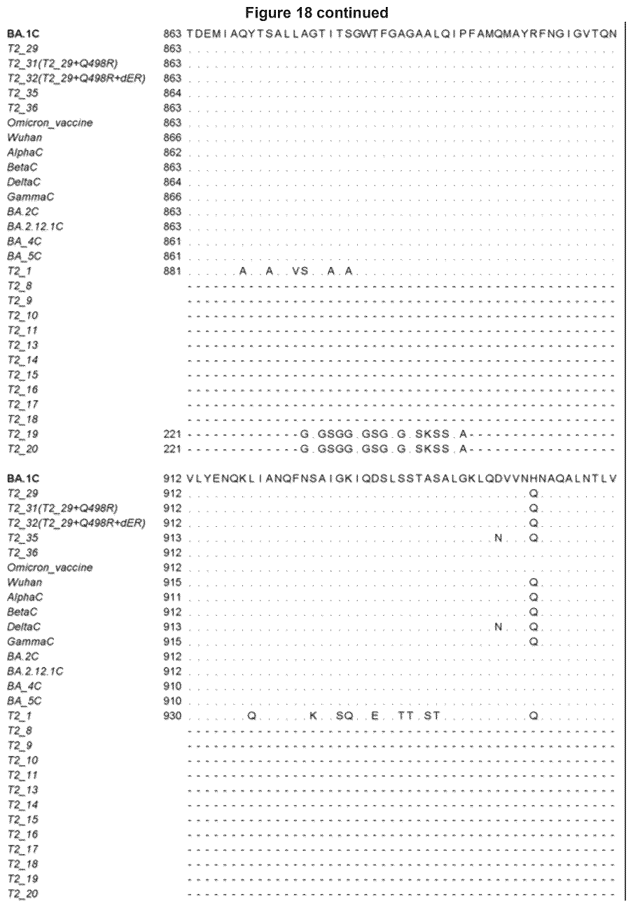

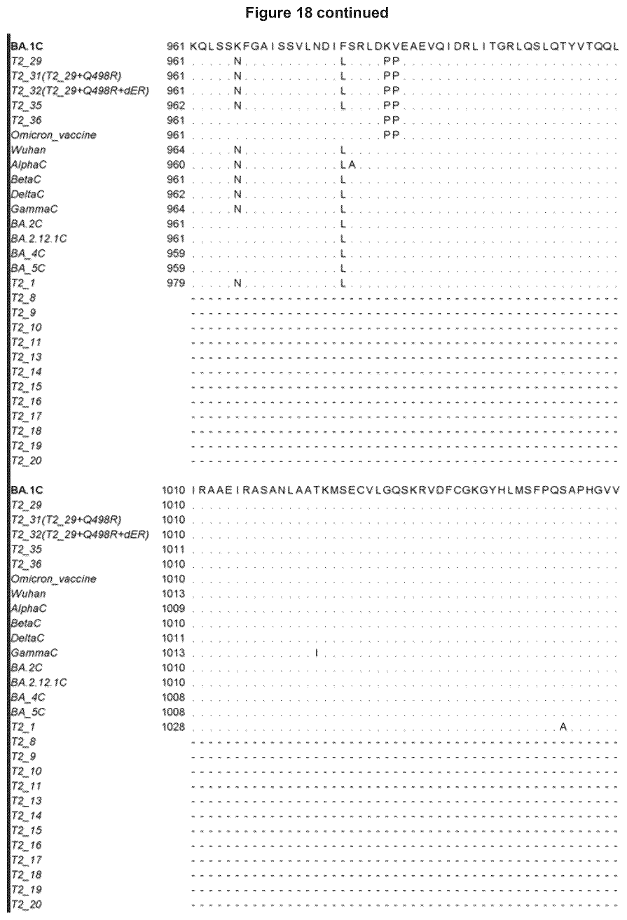

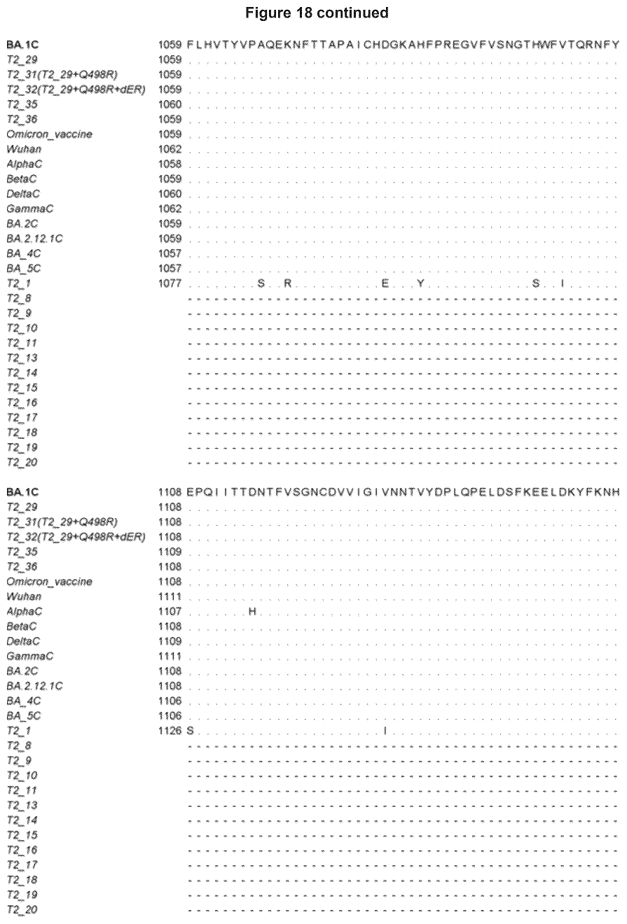

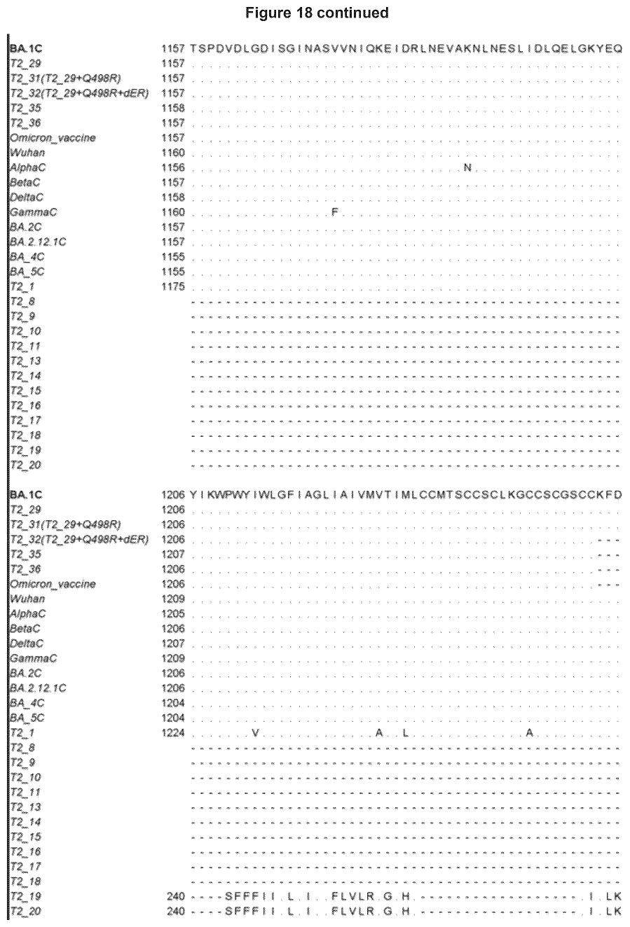

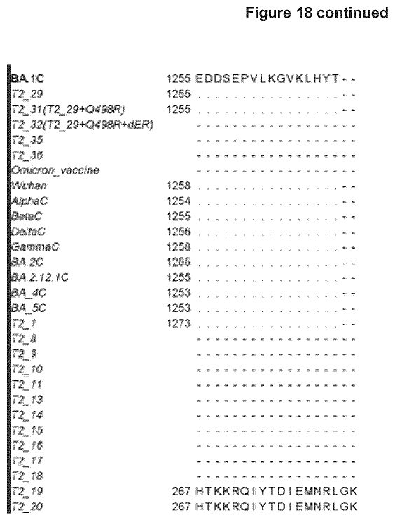

FIG. 18 shows an amino acid sequence alignment of designed S proteins CoV_S_T2_35(deom) (SEQ ID NO:1) and CoV_S_T2_36 (omide) (SEQ ID NO:2) with previously designed S protein sequence, RBD sequence, and reference sequences, with BA. 1 as the alignment reference sequence;

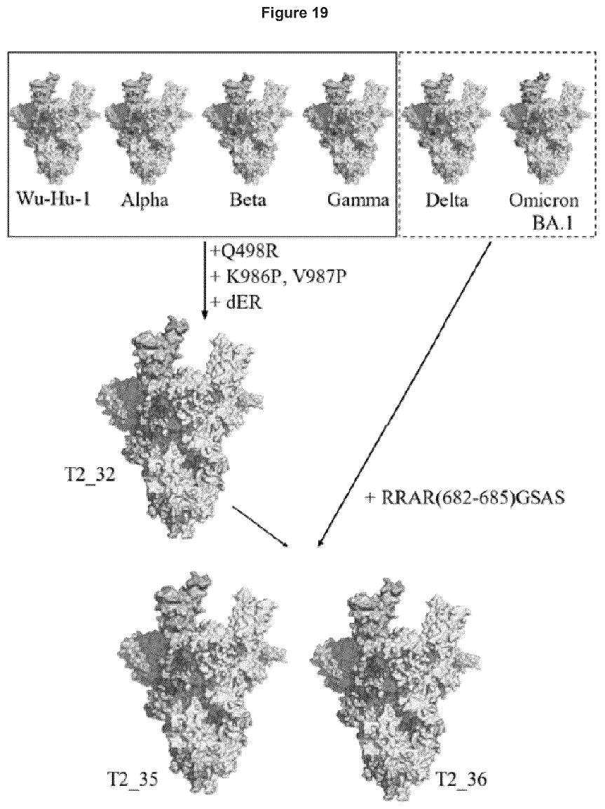

FIG. 19 shows in-silico design of the vaccine antigens T2_32 (also called CoV_T2_29+Q+dER), T2_35, and T2_36. Surface representation of the extra-virion region of the spike protein of SARS-CoV-2. The three subunits are coloured in pale yellow (middle subunit), pale blue (left-hand subunit), and grey (right-hand subunit). The mutations reported in different variants are coloured as red (shown as shaded dots on the representations of the variants). The mutations introduced in the spike vaccine antigens are coloured as orange in T2_32, T2_35, and T2_36 (shown as shaded dots in the designed antigens);

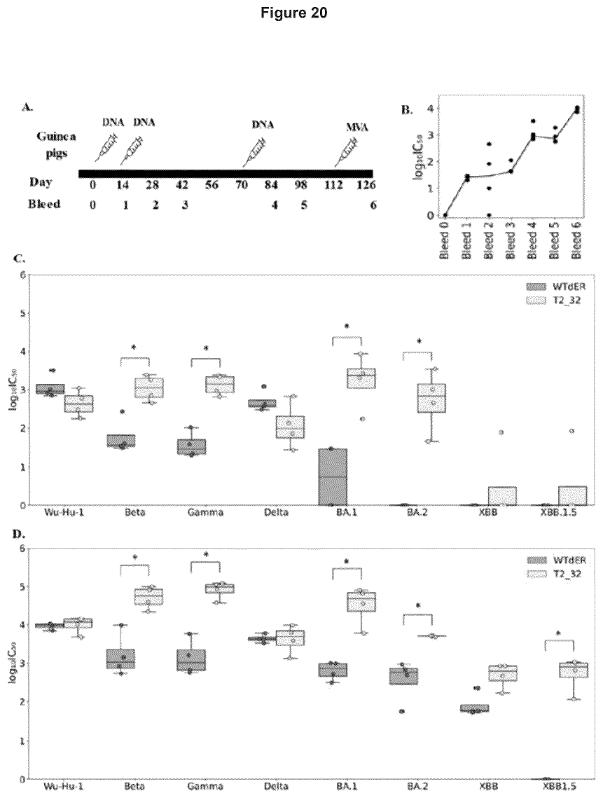

FIG. 20 shows an immunogenicity study of T2_32 (also called CoV_T2_29+Q+dER) in guinea pigs. A) Bleed and immunisation schedule in Guinea pigs (DNA prime followed by MVA boost). B) Distribution of the neutralisation titres against Wu-Hu-1 pseudotype on immunisation with WTdER. The x-axis represents the bleed number, and the y-axis represents the log₁₀(IC50) values. C) Distribution of the neutralisation titre of bleed 4 against Wu-Hu-1 and VOCs-Beta, Gamma, Delta, BA.1, BA.2, XBB, XBB.1.5. The x-axis represents the pseudoviruses tested for neutralisation, and the y-axis represents the log₁₀(IC50) values. The boxplots are colour coded according to vaccines and are in the following order from left-right for each PV tested: WTdER, T2_32. D) Distribution of the neutralisation titre of bleed 6 against Wu-Hu-1 and VOCs-Beta, Gamma, Delta, BA. 1, BA.2, XBB, XBB.1.5. The x-axis represents the pseudoviruses tested for neutralisation, and the y-axis represents the log₁₀(IC50) values. The boxplots are colour coded according to vaccines and are in the following order from left-right for each PV tested: WTdER, T2_32. Mann-Whitney U test is used as statistical significance test in all the plots (p-value: * ≤0.05, **<0.01, *** ≤0.001). The distributions that are not statistically significant are not labelled in the plot;

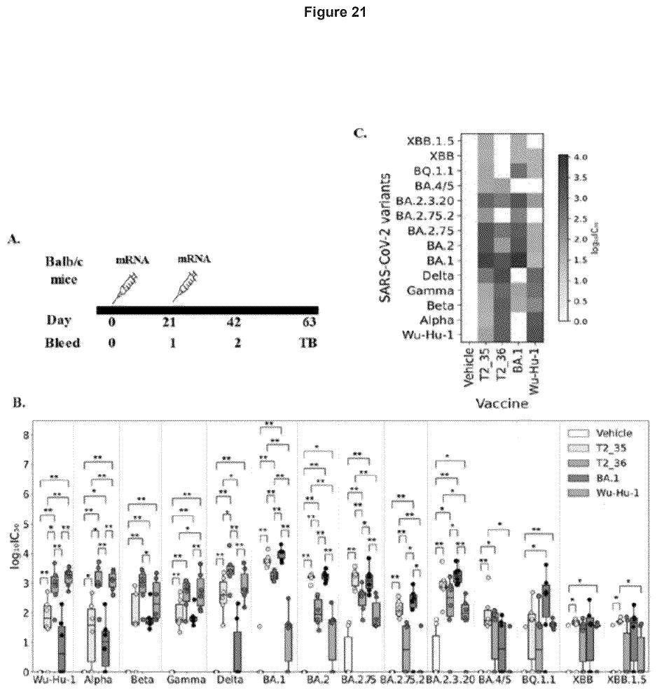

FIG. 21 shows an immunogenicity study of T2_35 and T2_36 in mice. A) Bleed and immunisation schedule of T2_35 and T2_36 mRNA vaccines in mice. B) Distribution of the neutralisation titre of terminal bleed against Wu-Hu-1 and VOCs. The x-axis represents the pseudoviruses tested for neutralisation, and the y-axis represents the log₁₀(IC50) values. The boxplots are colour coded according to vaccines, and are in the following order from left to right for each PV tested: vehicle, T2_35, T2_36, BA. 1, Wuhan. Mann-Whitney U test is used as statistical significance test in all the plots (p-value: * ≤0.05, **<0.01, *** ≤302 0.001). The distributions that are not statistically significant are not labelled in the plot. C) A heatmap representation of the median log₁₀(IC50) values for all the vaccines and SARS-CoV-2 variants tested for. The darker the shade the higher the log₁₀IC50. All the median values below 1.5 have been plotted as zero;

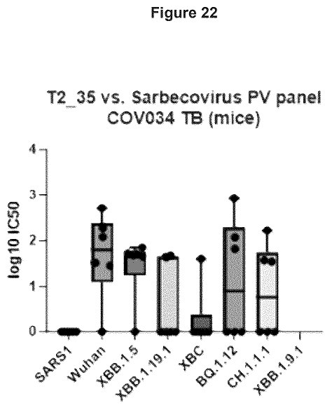

FIG. 22 shows an immunogenicity study of CoV_S_T2_35(Deom; SEQ ID NO: 1) in mice. Mice were immunised twice with mRNA at weeks 0 and 3, and bled at 3 week intervals beginning from day 0. Data shown uses sera from terminal bleed (6 weeks after boost). The x-axis represents the pseudoviruses tested for neutralisation, and the y-axis represents the log₁₀(IC50) values;

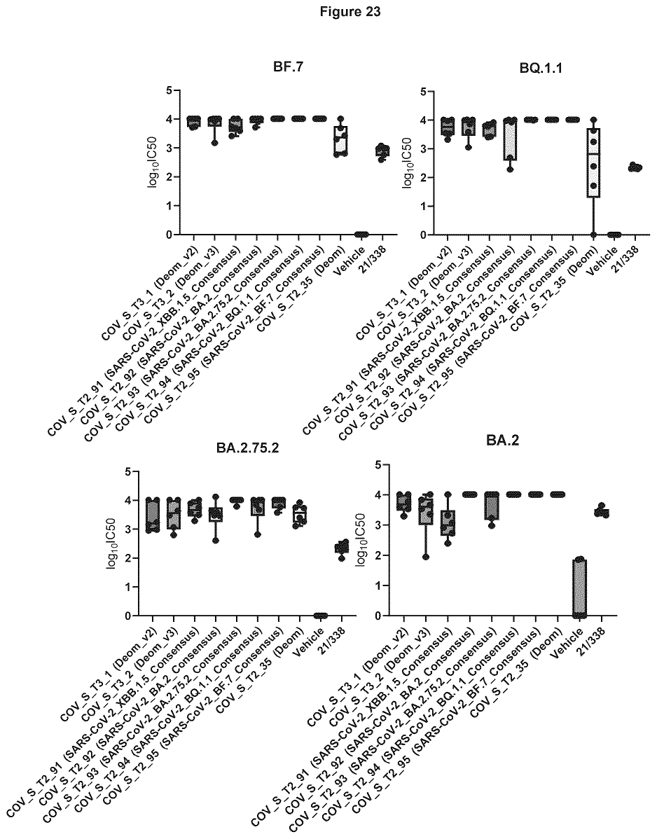

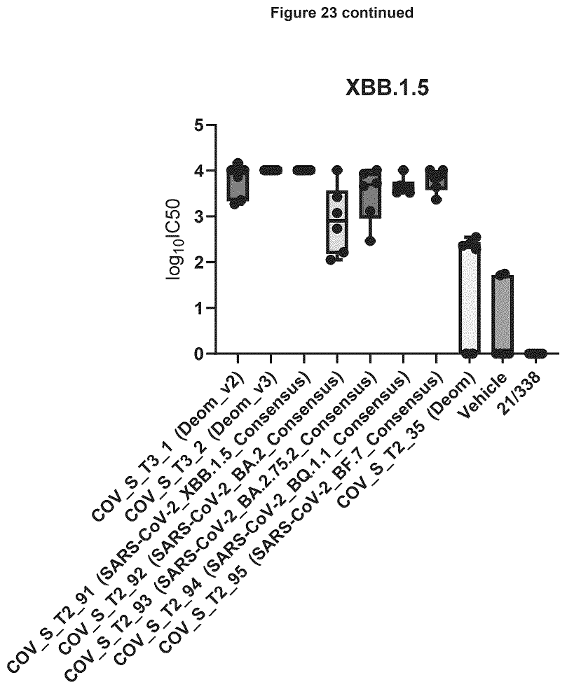

FIG. 23 shows immunogenicity study of optimised coronavirus ‘SuperSpike’ mRNA constructs CoV_S_T2_35(Deom; SEQ ID NO:1), CoV_S_T3_1(Deom_v2; SEQ ID NO:31) and CoV_S_T3_2(Deom_v3; SEQ ID NO:32) in mice. Mice were immunised with two doses of 10 μg mRNA in 100 μl vehicle, with a 3-week interval between doses. Mice were bled at 3 weeks after first dose and 6 weeks after first dose. Terminal bleed was 9 weeks after first dose. Sera was challenged by SARS-CoV-2 lentiviral pseudoviruses expressing VOC spike protein. The x-axis represents the pseudoviruses tested for neutralisation, and the y-axis represents the log₁₀(IC50) values.

TABLE of SEQ ID NOs:

| SEQ ID NO: | Description |
|---|---|
| 1 | CoV_S_T2_35 (deom) (designed S protein) |
| 2 | CoV_S_T2_36 (omide) (designed S protein) |
| 3 | Omicron_vaccine (designed S protein) |
| 4 | Wuhan_Hu_1 (Wuhan) |
| 5 | AlphaC |
| 6 | BetaC |
| 7 | GammaC |
| 8 | DeltaC |
| 9 | Omicron BA.1C |
| 10 | Omicron BA.2C |
| 11 | Omicron BA.2.12.1C |
| 12 | Omicron BA.4C |
| 13 | Omicron BA.5C |
| 14 | CoV_T2_1 |
| 15 | CoV_T2_8 (M7 RBD) |
| 16 | CoV_T2_9 (M8 RBD) |
| 17 | CoV_T2_10 (M9 RBD) |
| 18 | CoV_T2_11 (M10 RBD) |
| 19 | CoV_T2_13 (designed S protein RBD sequence) |
| 20 | CoV_T2_14 (designed S protein RBD sequence) |
| 21 | CoV_T2_15 (designed S protein RBD sequence) |
| 22 | CoV_T2_16 (designed S protein RBD sequence) |
| 23 | CoV_T2_17 (designed S protein RBD sequence) |
| 24 | CoV_T2_18 (designed S protein RBD sequence) |
| 25 | CoV_T2_19 (designed S protein RBD sequence) |
| 26 | CoV_T2_20 (designed S protein RBD sequence) |
| 27 | CoV_T2_29 (T2_29) |
| 28 | CoV_T2_31 (T2_29 + Q498R) |
| 29 | CoV_T2_32 (T2_29 + Q498R + dER) |
| 30 | CoV_S_T2_35 Scaffold Sequence |
| 31 | COV_S_T3_1(Deom_v2) |
| 32 | COV_S_T3_2(Deom_v3) |
| 33 | RBD portion of CoV_S_T2_35 Scaffold Sequence |
| 34 | RBD portion of CoV_S_T2_35(deom) |
| | (SEQ ID NO: 1) |
| 35 | RBD portion of COV_S_T3_1(Deom_v2) |
| | (SEQ ID NO: 31) |
| 36 | RBD portion of COV_S_T3_2(Deom_v3) |
| | (SEQ ID NO: 32) |

### EXAMPLE 1—VACCINE SEQUENCES

This example provides amino acid and nucleic acid sequences of full-length S-protein for embodiments of the invention known as CoV_S_T2_35(deom), CoV_S_T2_36 (omide), and Omicron_vaccine.

>COV_S_T2_35 (deom)
(SEQ ID NO: 1)
Amino acid sequence:
MFVFLVLLPLVSSQCVNLRTRTQLPPAYTNSFTRGVYYPDKVFRSSVLHSTQDLFLPFFSNVTWFHAIHVSGTNGTKRFDNPV
LPFNDGVYFASTEKSNIIRGWIFGTTLDSKTQSLLIVNNATNVVIKVCEFQFCNDPFLDVYYHKNNKSWMESGVYSSANNCTF
EYVSQPFLMDLEGKQGNFKNLREFVFKNIDGYFKIYSKHTPINLVRDLPQGFSALEPLVDLPIGINITRFQTLLALHRSYLTP
GDSSSGWTAGAAAYYVGYLQPRTFLLKYNENGTITDAVDCALDPLSETKCTLKSFTVEKGIYQTSNERVQPTESIVRFPNITN
LCPFGEVFNATRFASVYAWNRKRISNCVADYSVLYNSASFSTFKCYGVSPTKLNDLCFTNVYADSFVIRGDEVRQIAPGQTGN
IADYNYKLPDDFTGCVIAWNSNKLDSKVSGNYNYLYRLFRKSNLKPFERDISTEIYQAGNKPCNGVAGFNCYFPLRSYSFRPT
YGVGHQPYRVVVLSFELLHAPATVCGPKKSTNLVKNKCVNFNFNGLKGTGVLTESNKKFLPFQQFGRDIADTTDAVRDPQTLE
ILDITPCSFGGVSVITPGTNTSNQVAVLYQGVNCTEVPVAIHADQLTPTWRVYSTGSNVFQTRAGCLIGAEHVNNSYECDIPI
GAGICASYQTHTNSRGSASSVASQSIIAYTMSLGAENSVAYSNNSIAIPTNFTISVTTEILPVSMTKTSVDCTMYICGDSTEC
SNLLLQYGSFCTQLNRALTGIAVEQDKNTQEVFAQVKQIYKTPPIKDFGGFNFSQILPDPSKPSKRSFIEDLLFNKVTLADAG
FVKQYGDCLGDIAARDLICAQKFNGLTVLPPLLTDEMIAQYTSALLAGTITSGWTFGAGAALQIPFAMQMAYRFNGIGVTQNV
LYENQKLIANQFNSAIGKIQDSLSSTASALGKLQNVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQSAPHGVVFLHVTYVPAQEKNFTTAPAI
CHDGKAHFPREGVFVSNGTHWFVTQRNFYEPQIITTDNTFVSGNCDVVIGIVNNTVYDPLQPELDSFKEELDKYFKNHTSPDV
DLGDISGINASVVNIQKEIDRLNEVAKNLNESLIDLQELGKYEQYIKWPWYIWLGFIAGLIAIVMVTIMLCCMTSCCSCLKGC
CSCGSCC
>COV_S_T2_36 (omide)
(SEQ ID NO: 2)
Amino acid sequence:
MFVFLVLLPLVSSQCVNLTTRTQLPPAYTNSFTRGVYYPDKVERSSVLHSTQDLFLPFFSNVTWFHVISGTNGTKRFDNPVLP
FNDGVYFASIEKSNIIRGWIFGTTLDSKTQSLLIVNNATNVVIKVCEFQFCNDPFLDHKNNKSWMESEFRVYSSANNCTFEYV
SQPFLMDLEGKQGNFKNLREFVFKNIDGYFKIYSKHTPIIVREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQTSNFRVQPTESIVRFPNITNL
CPFGEVFNATRFASVYAWNRKRISNCVADYSVLYNSASFSTFKCYGVSPTKLNDLCFTNVYADSFVIRGDEVRQIAPGQTGNI
ADYNYKLPDDFTGCVIAWNSNNLDSKVGGNYNYRYRLFRKSNLKPFERDISTEIYQAGSKPCNGVEGFNCYFPLQSYGFQPTY
GVGYQPYRVVVLSFELLHAPATVCGPKKSTNLVKNKCVNFNFNGLTGTGVLTESNKKELPFQQFGRDIADTTDAVRDPQTLEI
LDITPCSFGGVSVITPGTNTSNQVAVLYQGVNCTEVPVAIHADQLTPTWRVYSTGSNVFQTRAGCLIGAEYVNNSYECDIPIG
AGICASYQTQTKSHGSASSVASQSIIAYTMSLGAENSVAYSNNSIAIPTNFTISVTTEILPVSMTKTSVDCTMYICGDSTECS
NLLLQYGSFCTQLKRALTGIAVEQDKNTQEVFAQVKQIYKTPPIKYFGGFNFSQILPDPSKPSKRSFIEDLLFNKVTLADAGF
IKQYGDCLGDIAARDLICAQKFKGLTVLPPLLTDEMIAQYTSALLAGTITSGWTFGAGAALQIPFAMQMAYRFNGIGVTQNVL
YENQKLIANQFNSAIGKIQDSLSSTASALGKLQDVVNHNAQALNTLVKQLSSKFGAISSVLNDIFSRLDPPEAEVQIDRLITG
RLQSLQTYVTQQLIRAAEIRASANLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAGLIAIVMVTIMLCCMTSCCSCLKGCC
SCGSCC
>Omicron_vaccine
(SEQ ID NO: 3)
Amino acid sequence:
MFVFLVLLPLVSSQCVNLTTRTQLPPAYTNSFTRGVYYPDKVFRSSVLHSTQDLFLPFFSNVTWFHVISGTNGTKRFDNPVLP
FNDGVYFASIEKSNIIRGWIFGTTLDSKTQSLLIVNNATNVVIKVCEFQFCNDPFLDHKNNKSWMESEFRVYSSANNCTFEYV
SQPFLMDLEGKQGNFKNLREFVEKNIDGYFKIYSKHTPIIVREPEDLPQGFSALEPLVDLPIGINITRFQTLLALHRSYLTPG
DSSSGWTAGAAAYYVGYLQPRTFLLKYNENGTITDAVDCALDPLSETKCTLKSFTVEKGIYQTSNFRVQPTESIVRFPNITNL
CPFDEVFNATRFASVYAWNRKRISNCVADYSVLYNLAPFFTFKCYGVSPTKLNDLCFTNVYADSFVIRGDEVRQIAPGQTGNI
ADYNYKLPDDFTGCVIAWNSNKLDSKVSGNYNYLYRLFRKSNLKPFERDISTEIYQAGNKPCNGVAGFNCYFPLRSYSFRPTY
GVGHQPYRVVVLSFELLHAPATVCGPKKSTNLVKNKCVNFNFNGLKGTGVLTESNKKFLPFQQFGRDIADTTDAVRDPQTLEI
LDITPCSFGGVSVITPGTNTSNQVAVLYQGVNCTEVPVAIHADQLTPTWRVYSTGSNVFQTRAGCLIGAEYVNNSYECDIPIG
AGICASYQTQTKSHGSASSVASQSIIAYTMSLGAENSVAYSNNSIAIPTNFTISVTTEILPVSMTKTSVDCTMYICGDSTECS
NLLLQYGSFCTQLKRALTGIAVEQDKNTQEVFAQVKQIYKTPPIKYFGGFNFSQILPDPSKPSKRSFIEDLLFNKVTLADAGF
IKQYGDCLGDIAARDLICAQKFKGLTVLPPLLTDEMIAQYTSALLAGTITSGWTFGAGAALQIPFAMQMAYRFNGIGVTQNVL
YENQKLIANQFNSAIGKIQDSLSSTASALGKLQDVVNHNAQALNTLVKQLSSKFGAISSVLNDIFSRLDPPEAEVQIDRLITG
RLQSLQTYVTQQLIRAAEIRASANLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAGLIAIVMVTIMLCCMTSCCSCLKGCC
SCGSCC

### EXAMPLE 2

### Residue Differences in Amino Acid Sequence of Influenza Tier 2 (T2) S Protein Vaccine Candidates CoV_S_T2_35, CoV_S_T2_36, and Omicron_Vaccine

This example identifies the amino acid residue differences in new designed sequences CoV_S_T2_35, CoV_S_T2_36, and Omicron_vaccine, compared to previous Tier 2 (T2)S-protein and RBD designs, and wild-type S-protein sequences. Alignment of the amino acid sequence of CoV_S_T2_35 and CoV-S_T2_36 with coronavirus S protein reference sequences SEQ ID NOs: 4-13, and previously designed vaccine sequences SEQ ID NOs: 14-29, is shown in FIG. 3. In particular, FIG. 3a shows the alignment of the sequences at positions wherein CoV_S_T2_35 comprises novel residues, and FIG. 3b shows the same for CoV_S_T2_36. FIGS. 17 and 18 show alignments of the sequences across the entire S protein sequence.

Table 3 below summarises novel amino acid residue changes in coronavirus S protein amino acid sequence for the embodiment of the invention known as CoV_S_T2_35 (SEQ ID NO:1). The amino acid residues at residue positions 680, 681, 683, 984, and 985, of CoV_S_T2_35, corresponding to residue positions 680, 681, 683, and 984-985, of DeltaC (SEQ ID NO:8), are novel. In particular, CoV_S_T2_35 comprises RRAR680-684GSAS, and KV984-985PP mutation, to stabilise the vaccine design. The designed sequence also comprises a C-terminal truncation, wherein 19 amino acid residues comprising the ER signal sequence have been deleted at residue positions 1253-1271 of DeltaC. This truncation improves surface expression of the antigen.

Table 6 below summarises novel amino acid residue changes in coronavirus S protein amino acid sequence for the embodiment of the invention known as CoV_S_T2_36 (SEQ ID NO:2). The amino acid residues at residue positions 679, 680, 682, 983, and 984, of CoV_S_T2_36, corresponding to residue positions 679, 680, 682, 983, and 984, of Omicron BA.1 (SEQ ID NO: 9), are novel. In particular, CoV_S_T2_36 comprises RRAR679-682GSAS, and KV983-984PP mutation, to stabilise the vaccine design. The designed sequence also comprises a C-terminal truncation, wherein 19 amino acid residues comprising the ER signal sequence have been deleted at residue positions 1252-1270 of Omicron BA.1C. This truncation improves surface expression of the antigen.

Omicron_vaccine comprises Omicron BA. 1 wild-type sequence with KV983-984PP mutation, to stabilise the vaccine design. The designed sequence also comprises a C-terminal truncation, wherein 19 amino acid residues comprising the ER signal sequence have been deleted at residue positions 1252-1270 of Omicron BA.1C. The designed sequence is used as a head-to-head comparison against CoV_S_T2_35 and CoV_S_T2_36 vaccine designs.

TABLE 3

| DeltaC (SEQ | DeltaC (SEQ | |
|---|---|---|
| ID NO: 8) | ID NO: 8) | CoV_S_T2_35 |
| residue | reference | (deom) SEQ ID |
| position | residue | NO: 1) |
| 680 | R | G |
| 681 | R | S |
| 683 | R | S |

TABLE 6

| Omicron | Omicron | |
|---|---|---|
| BA.1C | BA.1C | CoV_S_T2_36 |
| (SEQ ID | (SEQ ID | (omide) SEQ ID |
| NO: 9) | NO: 9) | NO: 2)/Omicron- |
| residue | reference | vaccine (SEQ ID |
| position | residue | NO: 3) |
| 679 | R | G |
| 680 | R | S |
| 682 | R | S |

The amino acid sequences of SARS2 S protein reference sequences:
Wuhan_Hu_1 (SEQ ID NO:4)
AlphaC (SEQ ID NO:5)
BetaC (SEQ ID NO:6)
GammaC (SEQ ID NO:7)
DeltaC (SEQ ID NO:8)
Omicron BA. 1C (SEQ ID NO:9)
Omicron BA.2C (SEQ ID NO: 10)
Omicron BA.2.12.1C (SEQ ID NO:11)
Omicron BA.4C (SEQ ID NO: 12)
Omicron BA.5C (SEQ ID NO:13)
   are shown below.

> NC_045512.2_Wuhan_Hu_1 (Wuhan)
(SEQ ID NO: 4)
Amino acid sequence:
MFVFLVLLPLVSSQCVNLTTRTQLPPAYTNSFTRGVYYPDKVERSSVLHSTQDLFLPFFS
NVTWFHAIHVSGTNGTKRFDNPVLPENDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVEKNIDGYFKIYSKHTPINLVRDLPQGESALEPLVDLPIGINITRFQT
LLALHRSYLTPGDSSSGWTAGAAAYYVGYLQPRTELLKYNENGTITDAVDCALDPLSETK
CTLKSFTVEKGIYQTSNERVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLERKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKELPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGENFSQILPDPSKPSKRSFIEDLLENKVTLADAGFIKQYGDC
LGDIAARDLICAQKENGLTVLPPLLTDEMIAQYTSALLAGTITSGWTFGAGAALQIPFAM
QMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKEDEDD
SEPVLKGVKLHYT
>AlphaC
(SEQ ID NO: 5)
Amino acid sequence:
MFVFLVLLPLVSSQCVNLTTRTQLPPAYTNSFTRGVYYPDKVERSSVLHSTQDLFLPFFSNVTWFHAI
SGTNGTKRFDNPVLPENDGVYFASTEKSNIIRGWIFGTTLDSKTQSLLIVNNATNVVIKVCEFQFCND
PFLGVHKNNKSWMESEFRVYSSANNCTFEYVSQPFLMDLEGKQGNEKNLREFVEKNIDGYFKIYSKHT
PINLVRDLPQGFSALEPLVDLPIGINITRFQTLLALHRSYLTPGDSSSGWTAGAAAYYVGYLQPRTEL
LKYNENGTITDAVDCALDPLSETKCTLKSFTVEKGIYQTSNERVQPTESIVRFPNITNLCPFGEVENA
TRFASVYAWNRKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDISTEIYQAGSTPC
NGVEGENCYFPLQSYGFQPTYGVGYQPYRVVVLSFELLHAPATVCGPKKSTNLVKNKCVNENENGLTG
TGVLTESNKKELPFQQFGRDIDDTTDAVRDPQTLEILDITPCSFGGVSVITPGTNTSNQVAVLYQGVN
CTEVPVAIHADQLTPTWRVYSTGSNVFQTRAGCLIGAEHVNNSYECDIPIGAGICASYQTQTNSHRRA
RSVASQSIIAYTMSLGAENSVAYSNNSIAIPINFTISVTTEILPVSMTKTSVDCTMYICGDSTECSNL
LLQYGSFCTQLNRALTGIAVEQDKNTQEVFAQVKQIYKTPPIKDFGGENFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRENGIGVTQNVLYENQKLIANQFNSAIGKIQDSLSSTASALGKLQDVVNQNA
QALNTLVKQLSSNFGAISSVLNDILARLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTHNTFVSGNCDVVIGIVNNTVYDPLQPELDSFKEELDKYFKNHT
SPDVDLGDISGINASVVNIQKEIDRLNEVANNLNESLIDLQELGKYEQYIKWPWYIWLGFIAGLIAIV
MVTIMLCCMTSCCSCLKGCCSCGSCCKFDEDDSEPVLKGVKLHYT
>BetaC
(SEQ ID NO: 6)
Amino acid sequence:
MFVFLVLLPLVSSQCVNLTTRTQLPPSYTNSFTRGVYYPDKVFRSSVLHSTQDLFLPFFSNVTWFHAI
HVSGTNGTKRFANPVLPENDGVYFASTEKSNIIRGWIFGTTLDSKTQSLLIVNNATNVVIKVCEFQFC
NDPFLGVYYHKNNKSWMESEFRVYSSANNCTFEYVSQPFLMDLEGKQGNEKNLREFVEKNIDGYFKIY
SKHTPINLVRGLPQGFSALEPLVDLPIGINITRFQTLHRSYLTPGDSSSGWTAGAAAYYVGYLQPRTE
LLKYNENGTITDAVDCALDPLSETKCTLKSFTVEKGIYQTSNERVQPTESIVRFPNITNLCPFGEVEN
ATRFASVYAWNRKRISNCVADYSVLYNSASFSTFKCYGVSPTKLNDLCFTNVYADSFVIRGDEVRQIA
PGQTGNIADYNYKLPDDFTGCVIAWNSNNLDSKVGGNYNYLYRLERKSNLKPFERDISTEIYQAGSTP
CNGVKGENCYFPLQSYGFQPTYGVGYQPYRVVVLSFELLHAPATVCGPKKSTNLVKNKCVNENENGLT
GTGVLTESNKKELPFQQFGRDIADTTDAVRDPQTLEILDITPCSFGGVSVITPGTNTSNQVAVLYQGV
NCTEVPVAIHADQLTPTWRVYSTGSNVFQTRAGCLIGAEHVNNSYECDIPIGAGICASYQTQTNSPRR
ARSVASQSIIAYTMSLGVENSVAYSNNSIAIPTNFTISVTTEILPVSMTKTSVDCTMYICGDSTECSN
LLLQYGSFCTQLNRALTGIAVEQDKNTQEVFAQVKQIYKTPPIKDFGGENFSQILPDPSKPSKRSFIE
DLLFNKVTLADAGFIKQYGDCLGDIAARDLICAQKENGLTVLPPLLTDEMIAQYTSALLAGTITSGWT
FGAGAALQIPFAMQMAYRENGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICHDGKAHFPR
EGVFVSNGTHWFVTQRNFYEPQIITTDNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIWLGFIAGLIAI
VMVTIMLCCMTSCCSCLKGCCSCGSCCKEDEDDSEPVLKGVKLHYT
>GammaC
(SEQ ID NO: 7)
Amino acid sequence:
MFVFLVLLPLVSSQCVNFTNRTQLPSAYTNSFTRGVYYPDKVERSSVLHSTQDLFLPFFSNVTWFHAI
HVSGTNGTKRFDNPVLPENDGVYFASTEKSNIIRGWIFGTTLDSKTQSLLIVNNATNVVIKVCEFQFC
NYPFLGVYYHKNNKSWMESEFRVYSSANNCTFEYVSQPFLMDLEGKQGNEKNLSEFVEKNIDGYFKIY
SKHTPINLVRDLPQGESALEPLVDLPIGINITRFQTLLALHRSYLTPGDSSSGWTAGAAAYYVGYLQP
RTFLLKYNENGTITDAVDCALDPLSETKCTLKSFTVEKGIYQTSNERVQPTESIVRFPNITNLCPFGE
VENATRFASVYAWNRKRISNCVADYSVLYNSASFSTFKCYGVSPTKLNDLCFTNVYADSFVIRGDEVR
QIAPGQTGTIADYNYKLPDDFTGCVIAWNSNNLDSKVGGNYNYLYRLERKSNLKPFERDISTEIYQAG
STPCNGVKGENCYFPLQSYGFQPTYGVGYQPYRVVVLSFELLHAPATVCGPKKSTNLVKNKCVNENEN
GLTGTGVLTESNKKELPFQQFGRDIADTTDAVRDPQTLEILDITPCSFGGVSVITPGTNTSNQVAVLY
QGVNCTEVPVAIHADQLTPTWRVYSTGSNVFQTRAGCLIGAEYVNNSYECDIPIGAGICASYQTQTNS
PRRARSVASQSIIAYTMSLGAENSVAYSNNSIAIPTNFTISVTTEILPVSMTKTSVDCTMYICGDSTE
CSNLLLQYGSFCTQLNRALTGIAVEQDKNTQEVFAQVKQIYKTPPIKDFGGENFSQILPDPSKPSKRS
FIEDLLENKVTLADAGFIKQYGDCLGDIAARDLICAQKENGLTVLPPLLTDEMIAQYTSALLAGTITS
GWTFGAGAALQIPFAMQMAYRENGIGVTQNVLYENQKLIANQFNSAIGKIQDSLSSTASALGKLQDVV
NQNAQALNTLVKQLSSNFGAISSVLNDILSRLDKVEAEVQIDRLITGRLQSLQTYVTQQLIRAAEIRA
SANLAAIKMSECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELDSFKEELDKYF
KNHTSPDVDLGDISGINASFVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT
>DeltaC
(SEQ ID NO: 8)
Amino acid sequence:
MFVFLVLLPLVSSQCVNLRTRTQLPPAYTNSFTRGVYYPDKVFRSSVLHSTQDLFLPFFS
NVTWFHAIHVSGTNGTKRFDNPVLPENDGVYFASIEKSNIIRGWIFGTTLDSKTQSLLIV
NNATNVVIKVCEFQFCNDPELDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLL
ALHRSYLTPGDSSSGWTAGAAAYYVGYLQPRTELLKYNENGTITDAVDCALDPLSETKCT
LKSFTVEKGIYQTSNERVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIADYN
YKLPDDETGCVIAWNSNNLDSKVGGNYNYRYRLFRKSNLKPFERDISTEIYQAGSKPCNG
VEGENCYFPLQSYGFQPTNGVGYQPYRVVVLSFELLHAPATVCGPKKSTNLVKNKCVNEN
FNGLTGTGVLTESNKKELPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYEC
DIPIGAGICASYQTQTNSRRRARSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVE
AQVKQIYKTPPIKDFGGFNFSQILPDPSKPSKRSFIEDLLENKVTLADAGFIKQYGDCLG
DIAARDLICAQKENGLTVLPPLLTDEMIAQYTSALLAGTITSGWTFGAGAALQIPFAMQM
AYRENGIGVTQNVLYENQKLIANQFNSAIGKIQDSLSSTASALGKLQNVVNQNAQALNTL
VKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQ
PELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKEDEDDSE
PVLKGVKLHYT
>Omicron BA.1C
(SEQ ID NO: 9)
Amino acid sequence:
MFVFLVLLPLVSSQCVNLTTRTQLPPAYTNSFTRGVYYPDKVFRSSVLHSTQDLFLPFFS
NVTWFHVISGTNGTKRFDNPVLPENDGVYFASIEKSNIIRGWIFGTTLDSKTQSLLIVNN
ATNVVIKVCEFQFCNDPFLDHKNNKSWMESEFRVYSSANNCTFEYVSQPFLMDLEGKQGN
FKNLREFVFKNIDGYFKIYSKHTPIIVREPEDLPQGFSALEPLVDLPIGINITRFQTLLA
LHRSYLTPGDSSSGWTAGAAAYYVGYLQPRTELLKYNENGTITDAVDCALDPLSETKCTL
KSFTVEKGIYQTSNERVQPTESIVRFPNITNLCPFDEVENATRFASVYAWNRKRISNCVA
DYSVLYNLAPFFTFKCYGVSPTKLNDLCFTNVYADSFVIRGDEVRQIAPGQTGNIADYNY
KLPDDFTGCVIAWNSNKLDSKVSGNYNYLYRLERKSNLKPFERDISTEIYQAGNKPCNGV
AGENCYFPLRSYSFRPTYGVGHQPYRVVVLSFELLHAPATVCGPKKSTNLVKNKCVNENE
NGLKGTGVLTESNKKELPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGENFSQILPDPSKPSKRSFIEDLLENKVTLADAGFIKQYGDCLGD
IAARDLICAQKFKGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNHNAQALNTLV
KQLSSKFGAISSVLNDIESRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT
>Omicron BA.2C
(SEQ ID NO: 10)
Amino acid sequence:
MFVFLVLLPLVSSQCVNLITRTQSYTNSFTRGVYYPDKVERSSVLHSTQDLFLPFFSNVT
WFHAIHVSGTNGTKRFDNPVLPENDGVYFASTEKSNIIRGWIFGTTLDSKTQSLLIVNNA
TNVVIKVCEFQFCNDPFLDVYYHKNNKSWMESEFRVYSSANNCTFEYVSQPFLMDLEGKQ
GNFKNLREFVFKNIDGYFKIYSKHTPINLGRDLPQGESALEPLVDLPIGINITRFQTLLA
LHRSYLTPGDSSSGWTAGAAAYYVGYLQPRTELLKYNENGTITDAVDCALDPLSETKCTL
KSFTVEKGIYQTSNERVQPTESIVRFPNITNLCPFDEVENATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYLYRLFRKSNLKPFERDISTEIYQAGNKPCNGV
AGENCYFPLRSYGFRPTYGVGHQPYRVVVLSFELLHAPATVCGPKKSTNLVKNKCVNENE
NGLTGTGVLTESNKKELPFQQFGRDIADTTDAVRDPQTLEILDITPCSEGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLENKVTLADAGFIKQYGDCLGD
IAARDLICAQKENGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNHNAQALNTLV
KQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT
>Omicron BA.2.12.1C
(SEQ ID NO: 11)
Amino acid sequence:
MFVFLVLLPLVSSQCVNLITRTQSYTNSFTRGVYYPDKVFRSSVLHSTQDLFLPFFSNVT
WFHAIHVSGTNGTKRFDNPVLPENDGVYFASTEKSNIIRGWIFGTTLDSKTQSLLIVNNA
TNVVIKVCEFQFCNDPELDVYYHKNNKSWMESEFRVYSSANNCTFEYVSQPFLMDLEGKQ
GNFKNLREFVFKNIDGYFKIYSKHTPINLGRDLPQGFSALEPLVDLPIGINITRFQTLLA
LHRSYLTPGDSSSGWTAGAAAYYVGYLQPRTELLKYNENGTITDAVDCALDPLSETKCTL
KSFTVEKGIYQTSNERVQPTESIVRFPNITNLCPFDEVENATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYQYRLERKSNLKPFERDISTEIYQAGNKPCNGV
AGENCYFPLRSYGFRPTYGVGHQPYRVVVLSFELLHAPATVCGPKKSTNLVKNKCVNENE
NGLTGTGVLTESNKKELPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENLVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGENESQILPDPSKPSKRSFIEDLLENKVTLADAGFIKQYGDCLGD
IAARDLICAQKENGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNHNAQALNTLV
KQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVELHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT
> Omicron BA.4C
(SEQ ID NO: 12)
Amino acid sequence:
MFVFLVLLPLVSSQCVNLITRTQSYTNSFTRGVYYPDKVERSSVLHSTQDLFLPFFSNVT
WFHAISGTNGTKRFDNPVLPENDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVEKNIDGYFKIYSKHTPINLGRDLPQGFSALEPLVDLPIGINITRFQTLLALH
RSYLTPGDSSSGWTAGAAAYYVGYLQPRTELLKYNENGTITDAVDCALDPLSETKCTLKS
FTVEKGIYQTSNERVQPTESIVRFPNITNLCPFDEVENATRFASVYAWNRKRISNCVADY
SVLYNFAPFFAFKCYGVSPTKLNDLCFTNVYADSEVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNENENG
LTGTGVLTESNKKELPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLENKVTLADAGFIKQYGDCLGDIA
ARDLICAQKENGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNHNAQALNTLVKQ
LSSKFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKEDEDDSEPVL
KGVKLHYT
>Omicron BA.5C
(SEQ ID NO: 13)
Amino acid sequence:
MFVFLVLLPLVSSQCVNLITRTQSYTNSFTRGVYYPDKVFRSSVLHSTQDLFLPFFSNVT
WFHAISGTNGTKRFDNPVLPENDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVEKNIDGYFKIYSKHTPINLGRDLPQGESALEPLVDLPIGINITRFQTLLALH
RSYLTPGDSSSGWTAGAAAYYVGYLQPRTELLKYNENGTITDAVDCALDPLSETKCTLKS
FTVEKGIYQTSNFRVQPTESIVRFPNITNLCPFDEVENATRFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNENENG
LTGTGVLTESNKKELPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLENKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNHNAQALNTLVKQ
LSSKFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKEDEDDSEPVL
KGVKLHYT

The amino acid sequences of previously designed Tier 2 (T2)S-protein and RBD designs:
CoV_T2_1 (SEQ ID NO:14)
CoV_T2_8 (SEQ ID NO:15)
CoV_T2_9 (SEQ ID NO:16)
CoV_T2_10 (SEQ ID NO: 17)
CoV_T2_11 (SEQ ID NO: 18)
CoV_T2_13 (SEQ ID NO: 19)
CoV_T2_14 (SEQ ID NO:20)
CoV_T2_15 (SEQ ID NO:21)
CoV_T2_16 (SEQ ID NO:22)
CoV_T2_17 (SEQ ID NO:23)
CoV_T2_18 (SEQ ID NO:24)
CoV_T2_19 (SEQ ID NO:25)
CoV_T2_20 (SEQ ID NO:26)
CoV_T2_29 (SEQ ID NO:27)
CoV_T2_31 (SEQ ID NO:28)
CoV_T2_32 (SEQ ID NO:29)
   are disclosed in the sequence listing for this application.

### EXAMPLE 3

### Digitally Immune Optimised Spike Vaccine Induces Broad Neutralising Responses Against SARS-CoV-2 Variants of Concern

### SUMMARY

Successive waves of SARS-CoV-2 variants of concern (VOC) have increased ability to escape existing immunity in vaccinated and infected populations. Current licensed SARS-CoV-2 vaccines have a reduced ability to elicit or boost neutralising antibodies against the most recent variants. New vaccine strategies are needed that can induce broad protective immunity across the VOCs. The evolution of SARS-CoV-2 variants can be re-capitulated from the detailed global surveillance efforts, and epidemiological sequence data on the VOCs. Using this data, we undertook a structure-based approach to computationally generate artificial Spike genes designed to induce neutralising antibody responses across a spectrum of VOCs. The first study was a VOC mutation informed spike termed T2_29, with multiple versions, such as a Q498R mutant, a mutation later to be acquired by the Omicron lineage VOCs and a C-terminal truncated and Q498R mutant. Three DNA immunisations in Guinea pigs between the

C-terminal truncated ancestral spike and T2_29 Spike with or without the C-terminal truncation and Q498R mutation, revealed superior immune response across the VOCs by T2_29 and modified T2_29 constructs in comparison to the C-terminal truncated ancestral construct. We further boosted all the groups with MVA expressing T2_29 with C-terminal truncated and Q498R modifications. MVA boost significantly increased the immune response across the groups against all the variants tested for. However, the immune response against Delta and Omicron was lower in comparison to other VOCs, so we designed second-generation vaccine antigens—T2_35 and T2_36. These were administered as mRNA in lipid nanoparticle formulation to mice to study the immune response against VOCs, particularly Delta and the emerging Omicron lineage variants. Superior broad neutralising capacity compared to mice immunised with the original Wuhan or Omicron BA. 1 spike immunogens were observed for both T2_35 and T2_36. These findings demonstrate the potential of novel computationally based; structure informed synthetic spike genes that are designed to induce pan-variant neutralising immune responses.

### INTRODUCTION

Over the past two years SARS-CoV-2 has acquired many spike mutations with different degree of effects on its interaction with the host, and its ability to escape pre-existing human immune responses acquired by vaccination and or infection. In addition to the evolution of SARS-CoV-2 in humans, it has been reported that the virus has transmitted to other mammals such as mink, cat, dogs, and certain species of deer. Cross-species infections of SARS-CoV-2, in which species-specific variants occur, provide an additional dimension to rate of evolution, their fitness, and immune escape features that may enable future SARS-CoV-2 variant epidemics. Since late 2020, many variants of concern (VOCs) have been reported, starting with the Alpha, Beta, Gamma, Delta, and the most recent variants of the Omicron lineage. It is the evolution of the spike protein, that enables immune escape and evasion that are influenced by several different selective pressures including immune pressure. The emergence of adaptive mutations present in the S protein can strongly affect host tropism and viral transmission. Facing an increasingly immune population, immunological escape from host immunity acquired during prior infection and/or vaccination is required for future variants to acquire advantageous changes allowing them to replicate and spread in the immune human population.

With the emergence of each subsequent variant of concern (VOC), there has been a declining level of vaccine induced neutralising antibodies induced by the ancestral Spike antigen (Wuhan Hu-1 strain) used by all the current generation of COVID-19 vaccines. Out of these, Delta, and Omicron, and Omicron sub-variants have been reported with higher transmission rates and immune escape from both naturally as well as vaccine acquired immunity. This necessitates an urgent update of current SARS-CoV-2 vaccines, which still use the ancestral strain. Continued use of vaccines based on ancestral sequence have a diminishing effect on facilitating de novo responses to new epitopes of new variants. Leading COVID-19 mRNA vaccine manufacturers have added an Omicron BA.1 Spike antigen to their Wuhan Spike based vaccine as a bivalent vaccine to provide better protection against the Omicron lineage of variants by adapting their vaccine to omicron lineage and administered either as a monovalent vaccine or a bivalent vaccine. Adapting the vaccine to a specific lineage can be beneficial to provide protection against a new emerging variant from the vaccine matched lineage but it may not provide desirable protection against emerging antigenically different lineages of SARS-CoV-2 or re-emergence of already reported antigenically distinct lineages of SARS-CoV-2. To circumvent this problem, we have developed next generation single Spike-based vaccine antigens that express diverse epitopes covering majority of the known VOCs. These novel vaccine antigens, T2_29, T2_35, and T2_36 demonstrated considerable neutralising breadth against SARS-Cov-2 pseudotypes expressing the ancestral Wuhan spike, Delta lineage as well as against the Omicron BA. 1, BA.2, and BA.4/5 variants.

### Methods

### In-Silico Design of the Vaccine Antigen

A consensus sequence was generated for each VOCs viz. Alpha, Beta, Gamma, Delta, and Omicron BA. 1 using the sequences deposited in NCBI virus. Each mutation was mapped to the different regions of Spike and clusters of mutation from different structural domains were sequentially combined to generate the next generation spike-based vaccine antigens. The structural integrity of the resultant vaccine antigens was checked for by generating homology models using Modeller software.

### Production and Transformation of Plasmids

Sequences of vaccine designs were gene-optimized and adapted to human codon use via the GeneOptimizer algorithm. These genes were cloned into pEVAC (GeneArt, Germany) via restriction digestion. Plasmids were transformed via heat-shock in chemically induced competent E. coli DH5a cells (Invitrogen 18265-017). Plasmid DNA was extracted from transformed bacterial cultures via the Plasmid Mini Kit (Qiagen 12125). All plasmids were subsequently quantified using UV spectrophotometry (NanoDrop™—Thermo Scientific).

### Vaccination Experiments in Guinea Pigs

Four groups of four seven-week-old female Hartley guinea pigs were purchased from Envigo (Maastricht, Netherlands). Guinea pigs were immunised at two-week intervals with 200 μg DNA vaccines bearing the antigen gene in the pURVac vector, administered by intradermal route using the Pharmajet© device in a total volume of 200 μl over the hind legs. Animals were given three doses of DNA by the same route and then boosted with MVA by intramuscular route at a dose of 1e7 PFU/dose seven weeks after the first three doses. Bleeds were taken through the saphenous vein at two weeks intervals.

### Vaccination Experiments in Mice

Five groups of six female 8-10-week-old BALB/c mice were purchased from Charles River Laboratories (Kent, United Kingdom). Mice were immunised twice at intervals of 3 weeks. The vaccine antigens—T2_35 and T2_36, modified Ancestral and modified Omicron BA. 1 were delivered by mRNA in lipid nanoparticle formulation. A total volume of 100 μl of PBS containing 10 μg of lipid encapsulated mRNA was administered intramuscularly over the two hind legs. The naïve mice group were administered 100 μl of PBS. Bleeds were taken at 3-weekly intervals.

### Production of Lentiviral Pseudotypes

Lentiviral pseudotypes were produced by transient transfection of HEK293T/17 cells with packaging plasmids p8.91 and pCSFLW and different spike expression plasmids bearing the using the Fugene-HD transfection reagent. Supernatants were taken after 48h, filtered at 0.45 μm and titrated on HEK293T/17 cells transiently expressing human ACE-2 and TMPRSS2. Target cells used were HEK293T/17 cells transfected 24 h prior with 2 μg huACE-2 and 75 ng TMPRSS2.

### Pseudotype-Based Micro-Neutralisation Assay

Pseudotype-based micro-neutralisation assay was performed as described previously. Briefly, serial dilutions of serum were incubated with SARS-CoV-2 spike bearing lentiviral pseudotype for 1 h at 37° C., 5% CO2 in 96-well white cell culture plates. 1.5×104 HEK293T/17 transiently expressing human ACE-2 and TMPRSS2 were then added per well and plates incubated for 48 hrs at 37° C., 5% CO2 in a humidified incubator. Bright-Glo (Promega) was then added to each well and luminescence read after a five-minute incubation period. Experimental data points were normalised to 100% and 0% neutralisation controls and non-linear regression analysis performed in GraphPad Prism 9 to produce neutralisation curves and IC50 values.

### Statistical Analyses

Two-tailed Mann-Whitney U tests were performed for all the comparisons using the Python sklearn package. All the plots were generated using the Python Matplotlib package.

### Results

### In-Silico Design of the Vaccine Antigen

The structure of the spike protein of SARS-CoV-2 can be antigenically divided into three distinct regions,—the N-terminal domain (NTD), the receptor binding domain (RBD), and the stalk region. The RBD possesses most of the experimentally characterised epitopes, followed by the NTD and the stalk. The relevance of these epitopes in protection from SARS-CoV-2 can also be appreciated from the observation of multiple mutations in the RBD and NTD in the SARS-CoV-2 VOCs. For our next generation variant vaccine antigen, we clustered all the reported mutations in the VOCs. Alpha, Beta, Gamma, Delta, and Omicron (BA. 1) into NTD, RBD, and stalk regions. It is important to note that for our bioinformatics analyses, we consider the epitopes in NTD, RBD, and stalk to be non-synergistic in eliciting immune responses and immune responses to these domains would be independent of each other. Once, epitopes were clustered, antigens were generated by sequential combination of different VOC specific mutations in NTD, RBD, and stalk. Only mutations that were reported in immunodominant regions were considered for the design. Based on these combinations, the first-generation Spike vaccine antigen-T2_29 (FIG. 4) was generated using available data on Alpha, Beta, and Gamma variants. The T2_29 modified Spike was further modified to three other antigens viz. T2_29+Q, and T2_29+Q+dER. The mutation Q498R was observed to be prominent in the circulating SARS-CoV-2 variants of interest prior to April 2021 and was included on the backbone of T2_29 to give T2_29+Q design as a pre-emptive antigen design for future variants. It is interesting to note that the Q498R mutation was later acquired by the Omicron variants in late 2021. A C-terminal deletion version of the T2_29+Q was also generated for comparison. Deletion of 19 amino acid from C-terminal was reported to express the spike protein on the surface of cell better in comparison to full-length and hence higher antigen presentation. We also deleted this C-terminal region from the WT ancestral antigen as a control, henceforth referred as WTdER. All these vaccine antigens have the stabilising double

Proline mutations, as reported in majority of the current vaccines. As our first-generation antigen—T2_29—was designed before the global outbreak of Delta and Omicron, we further generated our second-generation spike antigens—T2_35 and T2_36 (FIG. 4) to include these VOCs. In addition to the stabilising double Proline mutations, we also modified the Furin cleavage site RRAR to GSAS and introduced the C-terminal truncation.

First generation Spike vaccine antigen delivered by DNA and MVA in Guinea pigs Guinea pigs were immunised thrice with the antigens—T2_29, T2_29+Q, T2_29+Q+dER, and WTdER in DNA vector and were boosted with MVA expressing T2_29+Q+dER once (FIG. 5A). The neutralising titres were longitudinally analysed for WTdER against pseudoviruses (PVs) expressing VOC spikes. The neutralising antibodies peaked at bleed 4 following three immunisation and bleed 6 following MVA boost (FIG. 5B). The neutralising titre against all the VOCs and the ancestral sequence were measured for these bleeds (FIGS. 5C and 5D).

The first-generation spike vaccine antigen-T2_29 and its modifications viz. T2_29+Q, T2_29+Q+dER were able to induce broad neutralising response against all the VOCs tested. The T2_29 based antigens generated at least two-fold better neutralising response against Alpha, Beta, Gamma, and Omicron in comparison to WTdER (FIG. 5C) after three doses of DNA vaccine. The neutralising antibody titres against both the Ancestral sequence and Delta were comparable to WTdER (FIG. 5C) for T2_29 and T2_29+Q+dER. But a lower titre was observed for T2_29+Q before MVA boost. The WTdER generated a very weak neutralising antibody titre against the Omicron but all our vaccine antigens generated a robust neutralising antibody response against Omicron. Interestingly, the T2_29+Q showed lower neutralising titre to Omicron in comparison to T2_29 and T2_29+Q_dER. We believe the higher neutralising titre of the T2_29+Q_dER in comparison to T2_29+Q is due to higher expression of the construct. It is important to note that T2_29 doesn't include many of the mutations reported in Delta and Omicron variants, as these were designed prior to the outbreak of Delta and Omicron. Despite of lacking many of the important mutations reported in Delta and Omicron neutralising, T2_29 induced high titres against Omicron and titres comparable to wild type for Delta. We further boosted all the groups of guinea pigs with MVA expressing T2_29+Q+dER. We chose this specific construct, as this was antigenically closer to the omicron BA.1. On boosting with MVA, the neutralisation titre of all the vaccine antigens significantly increased (FIG. 5D). Most importantly, the neutralising titre of the WTdER against Omicron BA. 1 increased by 3-fold on boosting with MVA expressing T2_29+Q+dER. This strongly supports the applicability of these spike antigens for boosting the immune response against emerging variants in the already vaccinated or infected human population.

Next Generation Spike Vaccine Antigens Delivered by mRNA in Mice

Given that T2_29 generated superior responses to Alpha, Beta, Gamma, and Omicron variants but lower titre against Delta in comparison to wild type, a second-generation version of T2_29 was developed by including the mutations observed in Delta and Omicron BA. 1. We tested these second generation of modified spike vaccine antigens—T2_35 and T2_36—in mice. As Omicron BA. 1 has mutation in the majority of the epitopes reported in the NTD and RBD, we generated two sets of designs, using different combinations of mutations observed in Omicron to achieve a broader neutralising response against all the VOCs. As the current licensed booster vaccines are delivered as mRNA, we immunised mice with mRNA formulated in lipid nanoparticles. A prime-boost regime of three-weeks interval was followed (FIG. 6A). These second-generation Spike antigens were able to induce broad neutralising responses against Alpha, Gamma, Beta, Delta, and many of the important Omicron/Omicron-like lineages (FIGS. 6B and 6C, FIG. 7).

Although both T2_35 and T2_36 generated a robust neutralising titre against all the VOCs, a difference in titre was observed. For example, immunisation with T2_35 resulted in a large neutralising titre when challenged with Omicron variants, comparable to immunisation with BA. 1 vaccine (Omicron_vaccine), however resulted in a much higher titre against other VOCs than BA. 1 vaccine. While T2_36 generated a slightly lower neutralising titre against Omicron variants compared to BA. 1 vaccine, it resulted in a higher neutralising titre against other VOCs when compared with BA. 1 vaccine. The difference in the neutralising titre of our designs in comparison to BA. 1 vaccine and wild type can also be explained by the phylogenetic distance between the designs and all the known VOCs (FIG. 8). T2_35 is placed closer to the Omicron lineage while both the T2_29 and T2_36 are placed closer to Ancestral and rest of the VOCs. We also further checked the cross-neutralisation breadth of these mRNA vaccine against related sarbecovirus viz. SARS-1, WIV16, and RaTG13 (FIG. 7). Neutralising titre like Ancestral SARS-2 antigen was observed for T2_36 against WIV16 and RaTG13 but lower neutralising titre was observed for T2_35. No neutralisation was observed against SARS-1. For the related sarbecoviruses also, T2_36 showed higher neutralisation against the WIV16 and RaTG13 in comparison to T2_35, due to this closer similarity to Ancestral sequence. These observations validate our design strategy to design antigens that would generate a broad response against all the VOCs.

### DISCUSSION

Advancement in the field of vaccine technology and genomics had successfully led to the development and distribution of vaccine against COVID-19. These vaccines have been successful in controlling the spread of COVID-19 as well as the mortality rate due to COVID-19 but the rapid emergence of new variants of SARS-CoV-2 has caused worrying trend in the infection rate and associated hospitalization. The variants are observed to escape from immune response generated either by natural infection or vaccination, causing infections as well as re-infection. This has led to further boosting of the immune response by immunising the population with booster dose of the original vaccine. Although the booster results in an increase in the antibody titre, it still may be ineffective against emerging variants. In view of this, many vaccine manufacturing companies have rolled out vaccine with updated antigen to include the recent variants or combination of the original Wuhan-Hu-1 based antigen and the recent Omicron BA.1 variant. These may provide protection against the circulating variants but may fail against a new variant that will be phylogenetically different from the current circulating variants or if new variants that are phylogenetically alike to already known variants. Here we describe designs of novel spike-based antigens that incorporate the information on the mutations observed across the reported VOCs. We first designed our spike antigen using mutations observed in Alpha, Beta, and Gamma and validated the immunogenicity of the design in Guinea pigs using DNA/MVA vaccination regime. Robust neutralising titre were observed after three DNA vaccinations. T2_29 generated a superior neutralising response to all the tested VOCs except Delta, where it was comparable to the Ancestral Wuhan-Hu-1 antigen. Interesting and importantly, elicitation of comparable and superior immune response to Delta and Omicron BA. 1 by T2_29 is encouraging and validate our rationale that the novel spike antigens that include mutation information across the VOCs would be better vaccine antigens against emerging variants in comparison to a natural variant sequence. We further validated this rationale by incorporating mutation information from Delta and Omicron BA. 1 into T2_29 designed sequence, to generate our second-generation designs-T2_35 and T2_36. We validated the immunogenicity of these candidates as mRNA in mice. Both of the candidates T2_35 and T2_36 generated a robust neutralising response against all the tested VOCs. Broad neutralising response by our vaccine candidates in two animal models and across two platforms, support the superiority of these designs independent of the platforms and animal models. Overall, from the data presented below we can conclude that antigenically engineered Spike genes may induce superior immune breadth than combinations of original and variant Spike antigens or chimeric antigens.

### Supplementary Information

FIG. 6 shows terminal bleed neutralisation data in mice against ancestral, Omicron, and

Delta coronavirus challenge.

FIG. 6A shows bleed schedule of the mice. FIG. 6B shows the distribution of the neutralisation titre of bleed 2 against Ancestral and VOCs-BA.1, BA.2, BA.4/5, and Delta. The x-axis represents the pseudoviruses test for neutralisation, and the y-axis represents the log 10(IC50) values. The boxplots are colour coded according to vaccines. FIG. 6C shows Distribution of the neutralisation titre of terminal bleed against Ancestral and VOCs-BA. 1, BA.2, BA.4/5, and Delta. The x-axis represents the pseudoviruses test for neutralisation, and the y-axis represents the log 10(IC50) values. The boxplots are colour coded according to vaccines. Mann-Whitney U test is used as statistical significance test in all the plots (p-value:* ≤0.05, **<0.01, *** ≤0.001). The distribution that are not statistically significant are not labelled in the plot.

FIG. 7 shows terminal bleed neutralisation data in mice against coronavirus challenge. FIG. 7A shows the distribution of the neutralisation titre of terminal bleed against Ancestral and VOCs-Alpha, Beta, and Gamma. The x-axis represents the pseudoviruses test for neutralisation, and the y-axis represents the log 10(IC50) values. The boxplots are colour coded according to vaccines. FIG. 7B shows the distribution of the neutralisation titre of terminal bleed against SARS-1, WIV16, RaTG13, and Ancestral. The x-axis represents the pseudoviruses test for neutralisation, and the y-axis represents the log 10(IC50) values. The boxplots are colour coded according to vaccines. Mann-Whitney U test is used as statistical significance test in all the plots (p-value: * ≤0.05, **<0.01, *** ≤0.001). The distribution that are not statistically significant are not labelled in the plot.

### EXAMPLE 4

### Supplementary Information

Background: Current COVID-19 Vaccines are based on wild-type Spike SARS-CoV-2, from the original Wuhan sequence or the Omicron BA. 1 Spike variant.

Problem: As new variants continue to emerge, the use of “historical” Spike antigens from past waves of SARS-CoV-2 variants will have reduced benefit on prevention of the emergence of new variants. Thus repeated boosting of immune responses from past immunisations or infections (“Original Antigenic Sin”) may have a diminishing effect on facilitating de novo responses to new epitopes to prevent the emergence of new variants.

Objective 1: To determine if a single engineered SARS-CoV-2 Spike design expressing diverse epitopes is capable of inducing neutralization across the SARS-CoV-2 Variants of Concern (VOCs), and to increase the breadth of protective immunity that can be achieved by novel immunogens to protect against future SARS-CoV-2 variants.

Objective 2: To demonstrate that these effects can be mediated by different vaccine vector delivery systems (i.e. DNA, mRNA or MVA).

### First Generation DNA, T2 29 (“Super Spike”)

### Study 1a:

Neutralising antibody titres in outbred Guinea pigs after DNA immunisation with SARS-CoV-2 Spikes designed with VOC mutations.

### Study Design:

Group 1. a full spike construct based on the original Wuhan sequence of SARS-CoV-2 with a deletion of the ER retention signal

Group 2a: T2_29, Group 2b: T2_29+Q, Group 2c: T2_29+Q+dER mutation (all in combination with N501Y)

### Results:

FIG. 9 shows VOC RBD binding antibody levels of guinea pigs at bleed 4, shown by ELISA. The area under the curve (=AUC) calculated from the logarithmic dilution curves is plotted against the different vaccine constructs. A-F: Binding to each of the RBD variants is plotted. Overall signal strength varies between RBD variants and thus a comparison can only be made within one variant RBD. For each group the 4 individual values and the mean with 95% CI were plotted.

As shown, at bleed 4, sera of Guinea pigs immunised with the WT spike DNA construct demonstrated the second highest level of neutralisation (mean IC₅₀=1,438) against the PV carrying the homologous WT spike. Neutralisation against the alpha PV was higher than against the wildtype (mean IC₅₀=3,844).

When assayed against the VOCs delta (573), beta (94), gamma (46), and omicron BA. 1/BA.2 (15, 26), WTΔER shows a continuous decrease in mean nAb titres with almost no neutralisation against omicron. Compared with all three group 2 Super spikes (2a, 2b, 2c) with the group 1 WTΔER immunised group, there is a significant increase in mean IC₅₀ nAb values when assayed against beta (**, p<0.01), gamma (**, p<0.005), omicron BA.1 (*, p <0.05), and omicron BA.2 (*, p<0.05). T2_29+Q, one of the two constructs carrying the additional Q498R mutation, shows lower levels of neutralisation (mean IC₅₀=295) against omicron BA. 1 than T2_29 (mean IC₅₀=3,045).

The RBD of the T2_29 construct is identical to that of beta and almost identical to gamma with K417N instead of gamma's K417T. T2_29 shares three AA mutations with omicron and T2_29+Q (+/−AER) additionally includes omicron's Q498R, making them the genetically closest constructs to omicron in this study. The delta variant, on the other hand, carries two RBD mutations not found in the other VOC's (except T478K in BA.2) nor in any of the Super-spike designs. The delta RBD is therefore the most antigenically distant from the Super-spike constructs, especially those including Q498R.

FIG. 10 shows the distribution of the neutralisation titre of guinea pig serum (at bleed 4) against Ancestral and VOCs, after DNA immunisation using WT vaccine (WTdER) and T2_29 vaccine groups (2a, 2b, 2c; data combined). The x-axis represents the pseudoviruses test for neutralisation, and the y-axis represents the IC50 values. The WT vaccine appears on the left for each coronavirus pseudovirus, and the combined T2_29 vaccine appears on the right for each coronavirus pseudotype.

Discussion: The significant differences in mean IC₅₀ values between the combined T2_29 groups (2a, 2b, 2c) against the WTΔER group reveal a strong increase in neutralisation against beta, gamma, and omicron over the WTΔER immunised group. The IC₅₀ levels against Beta and Gamma are very similar.

Neither the effect of NTD mutations nor that of N417 versus T417 in the PVs could be observed. The T2_29 group 2's nAb activity against the WT and delta variant pseudovirus are still similar to those of the WTΔER group.

The addition of VOC mutations is not a “zero-sum game” where any gain in neutralisation against one variant leads to an equal loss against another.

The T2_29 groups reveal a strong increase in neutralisation against beta, gamma, and omicron over the WTΔER immunised group. The T2_29 group's nAb levels against the WT and delta variant PVs are still similar to those of the WTΔER group.

### Study 1b:

Neutralising antibody titres in MVA boosted DNA immunised Guinea pigs with MVA T2_29+Q+dER.

### Study Design:

Group 1. DNA delivered WT spike+dER, all boosted with MVA T2 29+Q+dER Group 2: DNA delivered group 2a, 2b, 2c, all boosted with MVA T2_29+Q+dER FIG. 11G shows an overview of 3×DNA and MVA boost immunisation and bleed schedule for Groups 1 and 2. Guinea pigs were immunised with plasmid DNA (Guinea pig icons with PharmaJet device shown in green) on days 0, 14, and 70. The fourth immunisation with MVA (Guinea pig with syringe) followed on day 113. Bleeds (blood drop icons) were taken before the start of the immunisations, 2 and 4 weeks after each immunisation, and at the point of sacrifice (Terminal bleed).

### Results:

FIG. 11A-F shows neutralisation data at bleed 6 for guinea pigs immunised with WT or designed DNA constructs and then boosted with MVA T2_29+Q+dER. The Figure shows neutralisation data for each vaccine construct when challenged with a panel of VOCs. The x-axis represents the pseudoviruses test for neutralisation, and the y-axis represents the IC50 values.

Group 1: Despite the heterologous boost with T2_29+Q+AER, the nAb levels of the WTΔER group at bleed 6 still strongly correlated with levels at bleed 4 (Spearman r=0.83, ****). No correlation (p >0.05) was found between the WTΔER group at bleed 6, T2_29+Q+AER group at bleed 4. Notably, the WT vaccinated group given a heterologous MVA T2_29+Q+AER boost was found to only partially expand variant neutralisation.

Group 2: As expected, the three group 2 (2a, 2b, 2c) MVA T2_29+Q+AER boosted groups show a very similar pattern of neutralisation as at bleed 4. T2_29 group neutralisation of BA. 1 pseudovirus was not boosted to the same degree as that of the beta and gamma pseudoviruses.

FIG. 5 shows a summary of the data for this example; spike vaccine antigen T2_29 delivered by DNA and MVA in Guinea pigs: FIG. 5A. Bleed schedule of the Guinea pigs.

FIG. 5B. Distribution of the neutralisation titre of the Guinea pigs against Ancestral virus pseudotype on immunisation with WTdER. The x-axis represents the bleed number, and the y-axis represents the log 10(IC50) values.

FIG. 5C. Distribution of the neutralisation titre of bleed 4 against Ancestral and VOCs-Beta, Gamma, Delta, and BA.1. The x-axis represents the pseudoviruses test for neutralisation, and the y-axis represents the log 10(IC50) values. The boxplots are colour coded according to vaccines, and appear in the following order from left to right for each challenge variant: WT dER, T2_29, T2_29+Q, and T2_29+Q+dER.

FIG. 5D. Distribution of the neutralisation titre of bleed 6 against Ancestral and VOCs-Beta, Gamma, Delta, and BA. 1. The x-axis represents the pseudoviruses test for neutralisation, and the y-axis represents the log 10(IC50) values. The boxplots are colour coded according to vaccines, and the vaccines appear in the same order as for FIG. 5C. Mann-Whitney U test is used as statistical significance test in all the plots (p-value: * ≤0.05, **<0.01, *** ≤0.001). The distribution that are not statistically significant are not labelled in the plot;

mRNA Delivery of NextGen Digitally Designed SARS-CoV-2 Spikes to Neutralise Broad Spectrum VOCs

Aim: Two demonstrate mRNA delivery of Digitally Immune Optimised Synthetic Spike genes compared to WT Wuhan and Omicron BA. 1 Spikes. All Spikes compared had the same proline stabilization modifications

Groups: (4 groups of age matched groups each of 6 Balb-c mice)
1) WT Spike
2) BA. 1 Spike
3) COV-S-T2_35
4) COV-S-T2_36)

Methods: Two different Digitally Immune Optimised Synthetic Spike genes (COV-S-T2_35 and COV-S-T2_36) were compared to WT Wuhan and Omicron BA. 1 Spikes delivered by mRNA, administered intramuscularly at 0 and 3 weeks in groups of 6 BALB/c mice. Assays: SARS-CoV-2 pseudotype neutralisation assays were performed against the original Wuhan, Delta and the Omicron BA. 1, BA.2, BA.2.12.1 and BA.4/BA.5 variants (FIGS. 12-14).

Findings: 3 weeks post 2^{nd} immunisation: Sera from mice immunised with Wuhan Spike had marked titres against Wuhan, slightly reduced titres to Delta, and weak neutralisation responses to all 4 Omicron variants tested. Similarly, sera from mice immunised with BA. 1 neutralised BA. 1, BA.2 and BA.2.12 well, but showed reduced neutralisation against Wuhan, Delta as well as BA.4/BA.5 pseudoviruses. In contrast, immunization with COV-S-T2_36 demonstrated significant neutralisation breadth of responses across all variants tested.

FIG. 12 shows statistical comparisons, COV-S-T2_36 vs BA. 1 spike Log IC50 neutralisation of virus x immunisation group.

FIG. 13 shows statistical comparisons between BA. 1 and BA.4/5 PV, all vaccine groups IC50 neutralisation values.

FIG. 14 shows all vaccine groups IC50 neutralisation values.

### CONCLUSIONS

1. Proof of Concept that single DIOS-generated spikes are alone able to induce broad neutralising responses.
2. Responses showed superior breadth or responses when tested against single wild-type Wuhan or BA. 1 Spike antigens proposed for use in human SARS-CoV-2 booster vaccine campaigns.
3. At 6 weeks (3 weeks post 2nd), S-T2_36 induced neutralising responses against BA.4/BA.5 were not significantly different, whereas a clear Wuhan/Omicron divide is seen for WT antigens. At 9 weeks against BA.4/BA.5, S-T2_35>S-T2_36>BA. 1.
3. Digitally, immune optimized SARS-CoV-2 Spike genes may induce superior immune breadth than combinations of wt Wuhan and variant Spike antigens.

### EXAMPLE 5

### Broadly Neutralising Immune Response Induced by T2_35 Against SARS-CoV-2 Variants of Concern Including Omicron Sub Variants

SARS-CoV-2 and SARS-like pseudoviruses were used as challenge viruses against sera from mice immunized with T2_35, T2_36, Wuhan Spike, and BA. 1 Spike (FIG. 15).

### Findings

Vaccine candidate antigen T2_35 generates expanded breadth of coverage against SARS-CoV-2 variants of concern including Omicron sub variants. T2_35 outperforms T2_36 in neutralisation across Omicron variants and subvariants (FIG. 15).

Wuhan and BA. 1 spike vaccine antigens elicit neutralising antibody responses that are generally restricted to pre- and post-Omicron variants respectively whereas T2_35 displays expanded breadth against the entire panel.

### EXAMPLE 6

### CoV_S_T2_35 Scaffold Sequence

SEQ ID NO:30 below shows a scaffold S protein sequence in which the amino acid sequence of the constant regions of the scaffold is provided, with each variable amino acid residue (i.e. amino acid residues which can be varied to provide antigen which induces neutralising immune response against new and/or future SARS-CoV-2 variants) represented with an X (shown underlined in the sequence below):

| | |
|---|---|
| MFVFLVLLPL VSSQCVNLRT RTQLPPAYTN SFTRGVYYPD KVFRSSVLHS TQDLFLPFFS | 60 |
| NVTWFHAIHV SGTNGTKRED NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNFRVQP TESIVRFPNI TNLCPFXEVF NATXFASVYA WNRKRISNCV | 360 |
| ADYSVXYNSA XFXXFKCYGV SPTKLNDLCF TNVYADSFVI RGXEVXQIAP GQTGNIADYN | 420 |
| YKLPDDFTGC VIAWNSNKLD SKXXGNYNYX YRLERKSXLK PFERDISTEI YQAGNKPCNG | 480 |
| VAGXNCYXPL XSYXFRPTYG VGHQPYRVVV LSFELLHAPA TVCGPKKSTN LVKNKCVNEN | 540 |
| FNGLKGTGVL TESNKKELPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHTNSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVF | 780 |
| AQVKQIYKTP PIKDFGGENF SQILPDPSKP SKRSFIEDLL FNKVTLADAG FVKQYGDCLG | 840 |
| DIAARDLICA QKFNGLTVLP PLLTDEMIAQ YTSALLAGTI TSGWTFGAGA ALQIPFAMQM | 900 |
| AYRENGIGVT QNVLYENQKL IANQFNSAIG KIQDSLSSTA SALGKLQNVV NQNAQALNTL | 960 |
| VKQLSSNEGA ISSVLNDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |

Examples of sequences provided herein which are covered by this scaffold sequence are SEQ ID: 1 (CoV_S_T2_35(deom)), SEQ ID NO:35 (CoV_S_T3_1(Deom_v2)), and SEQ ID NO:36 (CoV_S_T3_2(Deom_v3)).

FIG. 16 shows an amino acid sequence alignment of CoV_S_T2_35(deom) (SEQ ID NO: 1) with CoV_S_T3_1(Deom_v2) (SEQ ID NO:31), and CoV_S_T3_2(Deom_v3) (SEQ ID NO: 32). Differences between the sequences are shown as the boxed residues.

The residues at the variable positions in the CoV_S_T2_35(deom) (SEQ ID NO:1), CoV_S_T3_1(Deom_v2) (SEQ ID NO:31), and CoV_S_T3_2(Deom_v3) (SEQ ID NO:32) amino acid sequences are listed in the table below:

| | Residue at corresponding | | |
|---|---|---|---|
| Variable amino acid | position of | | |
| residue position of | CoV_S_T2_35(deom) | COV_S_T3_1(Deom_v2) | COV_S_T3_2(Deom_v3) |
| SEQ ID NO: 30 | (SEQ ID NO: 1) | (SEQ ID NO: 31) | (SEQ ID NO32) |
| 337 | G | D | H |
| 344 | R | R | T |
| 366 | L | L | I |
| 371 | S | P | P |
| 373 | S | F | F |
| 374 | T | A | A |
| 403 | D | N | N |
| 406 | R | S | S |
| 443 | V | V | P |
| 444 | S | G | S |
| 450 | L | R | L |
| 458 | N | K | K |
| 484 | F | P | P |
| 488 | F | F | S |
| 491 | R | Q | Q |
| 494 | S | G | G |

The amino acid sequences of SEQ ID NO:31 (CoV_S_T3_1) (Deom_v2) and SEQ ID NO:32 (CoV_S_T3_2) (Deom_v3) are shown below:

| COV_S_T3_1(Deom_v2) (SEQ ID NO: 31): | |
|---|---|
| MFVFLVLLPL VSSQCVNLRT RTQLPPAYTN SFTRGVYYPD KVFRSSVLHS TQDLFLPFFS | 60 |
| NVTWFHAIHV SGTNGTKRFD NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNERVQP TESIVRFPNI TNLCPFDEVE NATRFASVYA WNRKRISNCV | 360 |
| ADYSVLYNFA PFFAFKCYGV SPTKLNDLCF TNVYADSEVI RGNEVSQIAP GQTGNIADYN | 420 |
| YKLPDDFTGC VIAWNSNKLD SKVGGNYNYR YRLERKSKLK PFERDISTEI YQAGNKPCNG | 480 |
| VAGPNCYFPL QSYGFRPTYG VGHQPYRVVV LSFELLHAPA TVCGPKKSTN LVKNKCVNEN | 540 |
| FNGLKGTGVL TESNKKELPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHINSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVE | 780 |
| AQVKQIYKTP PIKDFGGENF SQILPDPSKP SKRSFIEDLL FNKVTLADAG FVKQYGDCLG | 840 |
| DIAARDLICA QKFNGLTVLP PLLTDEMIAQ YTSALLAGTI TSGWTFGAGA ALQIPFAMQM | 900 |
| AYRFNGIGVT QNVLYENQKL IANQFNSAIG KIQDSLSSTA SALGKLQNVV NQNAQALNTL | 960 |
| VKQLSSNEGA ISSVLNDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |
| COV_S_T3_2(Deom_v3) (SEQ ID NO: 32): | |
| MFVFLVLLPL VSSQCVNLRT RTQLPPAYTN SFTRGVYYPD KVFRSSVLHS TQDLFLPFFS | 60 |
| NVTWFHAIHV SGTNGTKRED NPVLPENDGV YFASTEKSNI IRGWIFGTTL DSKTQSLLIV | 120 |
| NNATNVVIKV CEFQFCNDPF LDVYYHKNNK SWMESGVYSS ANNCTFEYVS QPFLMDLEGK | 180 |
| QGNFKNLREF VFKNIDGYFK IYSKHTPINL VRDLPQGFSA LEPLVDLPIG INITRFQTLL | 240 |
| ALHRSYLTPG DSSSGWTAGA AAYYVGYLQP RTFLLKYNEN GTITDAVDCA LDPLSETKCT | 300 |
| LKSFTVEKGI YQTSNFRVQP TESIVRFPNI TNLCPFHEVE NATTFASVYA WNRKRISNCV | 360 |
| ADYSVIYNFA PFFAFKCYGV SPTKLNDLCF TNVYADSFVI RGNEVSQIAP GQTGNIADYN | 420 |
| YKLPDDFTGC VIAWNSNKLD SKPSGNYNYL YRLERKSKLK PFERDISTEI YQAGNKPCNG | 480 |
| VAGPNCYSPL QSYGFRPTYG VGHQPYRVVV LSFELLHAPA TVCGPKKSTN LVKNKCVNEN | 540 |
| FNGLKGTGVL TESNKKFLPF QQFGRDIADT TDAVRDPQTL EILDITPCSF GGVSVITPGT | 600 |
| NTSNQVAVLY QGVNCTEVPV AIHADQLTPT WRVYSTGSNV FQTRAGCLIG AEHVNNSYEC | 660 |
| DIPIGAGICA SYQTHTNSRG SASSVASQSI IAYTMSLGAE NSVAYSNNSI AIPTNFTISV | 720 |
| TTEILPVSMT KTSVDCTMYI CGDSTECSNL LLQYGSFCTQ LNRALTGIAV EQDKNTQEVF | 780 |
| AQVKQIYKTP PIKDFGGENF SQILPDPSKP SKRSFIEDLL FNKVTLADAG FVKQYGDCLG | 840 |
| DIAARDLICA QKENGLTVLP PLLTDEMIAQ YTSALLAGTI TSGWTFGAGA ALQIPFAMQM | 900 |
| AYRFNGIGVT QNVLYENQKL IANQFNSAIG KIQDSLSSTA SALGKLQNVV NQNAQALNTL | 960 |
| VKQLSSNFGA ISSVINDILS RLDPPEAEVQ IDRLITGRLQ SLQTYVTQQL IRAAEIRASA | 1020 |
| NLAATKMSEC VLGQSKRVDF CGKGYHLMSF PQSAPHGVVF LHVTYVPAQE KNFTTAPAIC | 1080 |
| HDGKAHFPRE GVFVSNGTHW FVTQRNFYEP QIITTDNTFV SGNCDVVIGI VNNTVYDPLQ | 1140 |
| PELDSFKEEL DKYFKNHTSP DVDLGDISGI NASVVNIQKE IDRLNEVAKN LNESLIDLQE | 1200 |
| LGKYEQYIKW PWYIWLGFIA GLIAIVMVTI MLCCMTSCCS CLKGCCSCGS CC | 1252 |

### EXAMPLE 7

### Digitally Immune Optimised Spike Vaccine Induces Broad Neutralising Responses Against SARS-CoV-2 Variants of Concern

### SUMMARY

Current SARS-CoV-2 vaccines fail to induce robust neutralising antibodies against the most recent variants of concern (VOC). Here we describe three novel in-silico designed spike-based antigens capable of inducing neutralising antibodies across a spectrum of SARS-CoV-2 VOC. Three sets of antigens utilising pre-Delta (T2_32, also called T2_29+Q+dER), and post-Delta sequence data (T2_35 and T2_36) were designed. T2_32 elicited superior neutralising responses against many VOCs compared to the spike derived from the early Wu-Hu-1 strain when delivered in a DNA prime boost regime. Further, heterologous boosting with the attenuated poxvirus MVA induced broader neutralising immune responses alone than DNA immunisation with T2_32 alone. T2_35 and T2_36 elicited superior broad neutralising capacity in mice in comparison to Omicron BA.1 sequence when delivered by mRNA. These findings demonstrate the potential of structure-informed computationally derived modifications of spike-based antigens for induction of pan-variant neutralising immune responses.

### INTRODUCTION

Since its emergence in late 2019, SARS-CoV-2 has acquired many mutations with different degree of effects on its interaction with the host, and its ability to escape pre-existing human immune responses acquired by vaccination and/or infection¹. In addition to the evolution of SARS-CoV-2 in humans, it has been reported that the virus has transmitted to other mammals such as mink, cat, dogs, and certain species of deer². Cross-species infections of SARS-CoV-2 add an additional dimension to the rate of evolution, fitness, and immune escape features that may enable future SARS-CoV-2 variant epidemics. Since late 2020, many variants of concern (VOCs) have been reported, starting with the Alpha, Beta, Gamma, Delta, and the recent variants of the Omicron lineage. It is primarily the evolution of the spike protein that enables immune escape and evasion. The emergence of adaptive mutations present in the spike protein can strongly affect host tropism and viral transmission^{1,3}. Facing an increasingly immune population, immunological escape from host immunity is a feature of SARS-CoV-2 variants to enable them to replicate and spread globally in the immune human population.

With the emergence of each subsequent variant of concern (VOC) or sub-variant, there has been a declining neutralising activity of antibodies induced by vaccines based on SARS-CoV-2 Wu-Hu-1 strain. Out of these, Delta, and Omicron sub-variants have been reported with higher transmission rates and immune escape from both naturally as well as vaccine acquired immunity⁴⁻⁶. This has necessitated continuous update of SARS-CoV-2 vaccines to match the prevalent circulating strains. Leading COVID-19 mRNA vaccine manufacturers have adapted their vaccine to omicron lineage and administered either as a monovalent vaccine or a bivalent vaccine⁷⁻⁹. Over the past one year, rapid evolution and divergence of the Omicron lineage has been observed, with multiple strains dominant at different geographic locations¹⁰. Under such circumstances of multiple dominant strains, adapting the vaccine to match a single strain may not be an optimal approach and may provide sub-par protection of the population from SARS-CoV-2, a situation already observed in field of Influenza vaccines¹¹.

Here, we propose that an antigen that expresses diverse epitopes covering the breadth of the most dominant VOCs would be a better alternative to adapting the vaccine to one of the circulating strains. Utilising the publicly available sequence data, we have designed three antigens—pre-Delta (T2_32, also called T2_29+Q_dER), and two post-Delta (T2_35 and T2_36). The T2_32 antigen was compared with the Wu-Hu-1 based Spike antigen in guinea pigs and T2_35 and T2_36 was compared against the Omicron BA. 1 Spike antigen in mice. All the three digitally immune optimised synthetic vaccine (DIOSynVax) Spike vaccine antigens demonstrated broader neutralisation profiles in comparison to wild-type antigens.

The superiority of neutralisation breadth against VOCs that subsequently emerged, following their design, underscores the importance of this class of digitally designed antigens for inclusion as next generation COVID-19 booster vaccine candidates.

### Methods

In-silico design of the vaccine antigens

A consensus sequence was generated for spike protein of VOCs-Alpha, Beta, and Gamma using the sequences deposited in NCBI Virus (February 2021) 12. Multiple sequence alignment (MSA) was generated for Wu-Hu-1, Alpha, Beta, and Gamma using MAFFT algorithm¹³. For each position along the length of the multiple alignment, non-matched amino acid to Wu-Hu-1 was mapped. In addition, the available epitope information from IEDB¹⁴ was mapped along the length of the MSA. Each epitope residues and the non-matched amino acid was clustered into the three structural domains-N-terminal domain (NTD), receptor binding domain (RBD), and stalk (S2) region. Clusters of non-matched amino acids from different structural domains and epitope regions were combined to generate the novel spike-based vaccine antigen. Further mutations—K986P^{15,16} and V987P^{15,16} and Q498R¹⁷ were introduced to the final design—T2_32.

Using the same design protocol, two designs-T2_35 and T2_36 were generated using MSA including the consensus Delta and Omicron BA. 1 (December 2021). In addition to a double proline mutation, these designs were further stabilised by replacing the Furin cleavage site with GSAS motif¹⁵. The structural integrity of the resultant vaccine antigens was checked for by generating homology models using Modeller software¹⁸. The endoplasmic retention (ER) signal was removed from all the designs, including wild type controls.

Production and transformation of plasmids Sequences of vaccine designs (T2_32 and Wu-Hu-1) were RNA- and codon-optimized for high level expression in human cells via the GeneOptimizer algorithm¹⁹. These genes were cloned into pEVAC (GeneArt/Thermofisher, Germany) via restriction digestion. Plasmids were transformed via heat-shock in chemically competent E. coli DH5a cells (Invitrogen 18265-017). Plasmid DNA was extracted from transformed bacterial cultures via the Plasmid Mini Kit (Qiagen 12125). The DNA plasmids were purified using the EndoFree Plasmid Mega kit (Qiagen, Hilden, Germany) according to the manufacturer's instructions. Plasmids were quality controlled by sequencing and quantified using UV spectrophotometry (NanoDrop™—Thermo Scientific) and assessed for absence of endotoxin.

### Production of MVA

The MVA strain used in this study was MVA-CR19²⁰. Recombinant MVA that expresses T2_32 was generated as described previously²¹. Briefly, pure recombinant viruses were obtained by sequential plaque purification under agarose overlays and confirmed to be free of contaminating parental MVA-CR19 TK-GFP by PCR screening. MVA-CR19 (rMVA) encoding T2_32 was plaque purified for additional three rounds. The resulting recombinant MVA-CR19 T2_32 (MVA T2_32) virus stock was produced in suspension AGE1.CR.plX cells, purified sequential by ultra centrifugation and titrated on DF-1 cells. The absence of revertant or parental MVA and T2_32 insert identity was confirmed by PCR amplification and Sanger sequencing. Correct expression of T2_32 was confirmed by Western blot analysis with monoclonal antibody CR3022 (Invivogen, Toulouse, France) with cell lysates from HEK293 cells harvested 24 hours after infection (MOI 2) with MVA T2_32.

### Vaccination Experiments in Guinea Pigs

Two groups of four seven-week-old female Hartley guinea pigs were purchased from Envigo (Maastricht, Netherlands). Guinea pigs were immunised at 14 days intervals with 200 μg DNA vaccines bearing the antigen gene in the pEVAC vector, administered by intradermal route using the Pharmajet© device in a total volume of 200 μl over the hind legs. A third dose of DNA was administered at day 70 post prime and at day 112 was boosted intramuscular. With MVA encoding T2_32 with 2.0E7 PFU/dose. Bleeds were taken through the saphenous vein at two weeks intervals.

Synthesis and Packaging of mRNA

mRNA sequences encoding the sequences of the vaccine antigens (T2_35, T2_36, and Omicron BA. 1) were synthesized by in vitro transcription (IVT) from linearized plasmid DNA templates using modified nucleotides to generate partial modified mRNAs. After IVT, mRNAs were dephosphorylated and enzymatically polyadenylated. Purification steps were performed by precipitation and subsequently formulated in water for injection at a concentration of 1 mg/mL. mRNAs were stored at −80° C. until LNP encapsulation. Each mRNA was LNP encapsulated via nanoprecipitation by microfluidic mixing of mRNA in citrate buffer (pH 4.5) with ionizable-, structural-, helper- and polyethylene glycol (PEG) lipids in ethanol, followed by buffer exchange and concentration via tangential flow filtration. mRNA LNPs were filtered through a 0.2 μm membrane and stored at −20° C. until use. The drug product was analytically characterized, and the products were evaluated as acceptable for in vivo use.

### Vaccination Experiments in Mice

Five groups of six female 8-10-week-old BALB/c mice were purchased from Charles River Laboratories (Kent, United Kingdom). Mice were immunised twice at an interval of 21 days. A total volume of 100 μl of PBS containing 10 μg of lipid encapsulated mRNA encoding the antigens was administered intramuscularly over the two hind legs. The naïve mice group were administered 100 μl of PBS. Bleeds were taken 3 weeks after each immunisation, and a final bleed 6 weeks after the second immunisation.

### Production of Lentiviral Pseudotypes

Lentiviral pseudotypes were produced by transient transfection of HEK293T/17 cells with packaging plasmids p8.91^{22,23} and pCSFLW²⁴ and different SARS-CoV-2 VOC spike-bearing expression plasmids in the pEVAC backbone, using the Fugene-HD (Promega E2311) transfection reagent^{25,26}. Supernatants were harvested after 48h, passed through a 0.45 μm cellulose acetate filter, and titrated on HEK293T/17 cells transiently expressing human ACE- and TMPRSS2. Target HEK293T/17 cells were transfected 24 h prior with 2 μg pCAGGS153 huACE-2 and 150 ng pCAGGS-TMPRSS2 in a T75 tissue culture flask^{27,28}.

### Pseudotype-Based Micro-Neutralisation Assay

Pseudotype-based micro-neutralisation assays (pMN) were performed as described Previously²⁹. Briefly, serial dilutions of serum were incubated with lentiviral pseudotypes bearing SARS-CoV-2, and SARS-CoV-2 VOC spikes for 1 h at 37° C., 5% CO2 in 96-well white cell culture plates. 1.5×10⁴ HEK293T/17 cells transiently expressing human ACE-2 and TMPRSS2 were then added per well and plates incubated for 48 hrs at 37° C., 5% CO2 in a humidified incubator. Bright-Glo (Promega) was then added to each well and luminescence read after a five-minute incubation period. Experimental data points were normalised to 100% and 0% using neutralisation controls and non-linear regression analysis performed in GraphPad Prism 9 to produce neutralisation curves and IC50 values.

### Statistical Analyses

Two-tailed Mann-Whitney U tests were 167 performed for all the pairwise comparisons using the Python sklearn package³⁰. All the plots were generated using the Python Matplotlib package and statannotat package³¹.

### Animal Work Ethics

All animal work was approved by the Home Office under project licence P8143424B and approved by the Animal Welfare Ethical Review Body (AWERB).

### Results

### In-Silico Design of the Antigens

The structure of the spike protein of SARS-CoV-2 can be antigenically divided into three distinct regions,—the N-terminal domain (NTD), the receptor binding domain (RBD), and the stalk (S2) region. The RBD possesses most of the experimentally characterised epitopes, followed by the NTD and the stalk. The frequent emergence of multiple mutations in the RBD and NTD of the SARS-CoV-2 VOCs suggest that these epitopes are important targets for protective immune mechanisms. Computational antigen designs involved mapping the observed mutations in the VOCs-Alpha, Beta, and Gamma, onto the spike protein, then mapping these mutations as epitope regions using the IEDB¹⁴ database, and classified as immunodominant epitopes using the peptides reported in literature³². All the mutations in immunodominant regions were clustered based on both the VOC it has been observed in and the region it corresponds to in the spike protein.

Beta and Gamma variants have the same mutations in the immunodominant region of the RBD (E484K, N501Y) except K417N/T. In addition to the RBD region, the Gamma variant also has mutations in the reported immunodominant region of NTD (L18F, T20N, P26S). As mutations in Gamma variant was reported in two immunodominant regions, set of mutations reported in NTD was considered for the design. Based on the clustering of mutations—set of mutations reported in NTD for Alpha (69HΔ, 70VΔ, 144YΔ) and Gamma (L18F, T20N, P26S) variants, set of mutations reported in RBD for Beta (K417N, E484K) variant, set of mutations reported in S2 for Alpha (P681H) variant and the common set of mutations—N501Y and D614G were considered for the design. Another mutation—Q498R was also reported by Schreiber group as the most potent immune escape and high affinity mutation using in-vitro evolution¹⁷. This mutation was included in our design as a pre-emptive step. Interestingly, this mutation was later observed in the Omicron lineage by late 2021. In addition, K986P^{15,16}, V987P^{15,16}, and deletion of 19 amino acids from C-terminal (dER) were introduced to stabilise the antigen-T2_32 (FIG. 19). Deletion of 19 amino acid from the C-terminal was reported to increase the expression of the spike protein on the surface of cell in comparison to full-length³³ and hence leading to higher antigen presentation. This antigen was evaluated in DNA-MVA prime-boost regime in guinea pigs.

T2_32 was designed before the global outbreak of Delta and Omicron variants. As the later was reported to escape previous immune responses, attributable to having accumulated the most number of reported mutations^{5,34}, we designed another set of antigens as pre-emptive measure and tested these designs as mRNA antigens in mice. As Omicron has multiple distinct mutations in multiple immunodominant regions in comparison to T2_32 and the Delta variant, we combined the set of mutations on the backbone of T2_32 in two ways-(a) NTD and the S2 regions were enriched with the mutation observed in the Delta variant and RBD region was enriched with the mutations observed in Omicron BA. 1 variant-T2_35 and, (b) NTD and the S2 regions were enriched with the mutation observed in the Omicron BA. 1 variant and the RBD region was enriched with the mutations observed in Delta variant-T2_36 (FIG. 19). This was to ensure all the important immunodominant regions are represented for all the important VOCs. In addition to all the other stabilising mutations in T2_32, the Furin cleavage site was knocked-out (682RRAR to GSAS) 15 in these two antigens.

### Spike Vaccine Antigen (T2_32) Delivered by DNA and MVA in Guinea Pigs.

Guinea pigs were immunised thrice with the antigens—T2_32, and the dER version of Wu-Hu-1 spike in a pEVAC plasmid and boosted with MVA.CR19 expressing T2_32 once (FIG. 20A). The neutralising titres were longitudinally analysed for WTdER against pseudoviruses (PVs) expressing VOC spikes. The neutralising antibodies peaked at bleed 4 following three immunisation and bleed 6 following MVA boost (FIG. 20B). The neutralising titre against all the VOCs and the Wu-Hu-1 strain were measured for these bleeds (FIGS. 20C and 20D). We also included some of the recent VOCs to test the robustness of T2_32.

Both the antigens induced neutralising titres against Beta, Gamma, and Delta variants, after three DNA immunisations (FIG. 20C). High neutralising titre against BA.1 and BA.2 were observed only in guinea pigs immunised with T2_32 (FIG. 20C). Against the recent variants-XBB and XBB.1.5, only one of the guinea pigs immunised with T2_32 generated neutralising titre (FIG. 20C). Except against Wu-Hu-1 and Delta strain, T2_32 generated at least a log higher titre than the WTdER antigen. For both Wu-Hu-1 and Delta strains, the titres were comparable for both the antigens. As higher titres were observed in the group immunised with T2_32, we boosted both the group of guinea pigs with heterologous vector-MVA expressing T2_32 MVA has been shown as a promising heterologous boost to DNA, resulting in increased neutralising titres²¹. On boosting with MVA expressing T2_32, higher neutralising titres of at least a log-fold for both groups of guinea pigs across the entire VOC panel (FIG. 20D) was observed. Most importantly, significantly higher titres were observed for three of the Omicron variants-BA. 1, BA.2, and XBB for both the groups of guinea pigs, as well as significant high titres were for XBB. 1.5 in groups primed with T2_32. Taken together, these results show that our T2_32-design is far superior to Wu-Hu-1 based Spike antigens and proved to be future proofed to many of the VOCs.

Spike Vaccine Antigens (T2_35 and T2_36) Delivered by mRNA in Mice.

While the T2_32 study was ongoing, omicron variants became globally dominant in the human population. Omicron variants were reported to show higher resistance to sera from patients as well as majority of the known therapeutic antibodies³⁵. As a pre-emptive measure we designed two more antigens—T2_35 and T2_36 and formulated them in mRNA. The dER version of Omicron BA.1 and Wu-Hu-1 sequence was used as control in the study. A prime-boost regime of three-weeks interval was followed (FIG. 21A). These spike antigens were able to induce broad neutralising responses against Alpha, Beta, Gamma, Delta, and many of the important Omicron/Omicron-like lineages (FIGS. 21B and 21C).

Both T2_35 and T2_36 generated a robust neutralising titre against all the VOCs, but a distinct pattern was observed for T2_35 and T2_36 against pre- and post-Omicron VOCs. The former generated higher neutralising titres against VOCs from BA.1 forward into the omicron sub lineages, while T2_36 had higher neutralising titre neutralising antibodies to pre-omicron VOCs-Alpha through to Delta (FIGS. 21B and 21C). This difference in the neutralising titre can be rationalized by the sets of mutation represented in the designs. T2_35 is enriched with mutations observed in Omicron BA. 1 variant in the RBD region while T2_36 is enriched with mutations observed in Delta variant. This data supports the immunodominance epitopes in the RBD region over the NTD or S2 regions.

Interestingly, the T2_35 candidate generated neutralising titres to all the Omicron variants comparable to or which developed after BA. 1, but a significantly higher titre to Delta than BA. 1. Although T2_36 generated lower neutralising titres against Omicron variants compared to BA. 1, it significantly induced higher neutralising antibody titres compared to the BA. 1 response against rest of the VOCs. The neutralising titres for all the Omicron VOCs were higher for T2_36 in comparison to the Wu-Hu-1 spike-based antigen. To capture the breadth and potency of the T2_35 and T2_36 in comparison to Wu-Hu-1 and BA. 1 spike-based antigen, we plotted the median log₁₀ IC₅₀ values (all median values below 1.5 values were scored as zero) for all the pseudoviruses tested in our panel (FIG. 21C). T2_35 induced the highest coverage with moderate potency across all VOCs. Neutralisation data for T2_35 against further, more recent VOCs, is plotted in FIG. 22.

### DISCUSSION

Advances in genomic tracking of viral epidemics and as witnessed during the COVID-19 pandemic have enabled the real-time assessment of vaccines to neutralise emerging viral variants. Here we leverage the tracking of pathogen genomics to digitally design next generation vaccine antigens for COVID-19 with improved neutralisation capacity against rapidly emerging variants. While the current licensed vaccines have been instrumental in alleviating the severity of COVID-19 disease, continued transmission with the rapid emergence of new variants of SARS-CoV-2 are still cause for a worrying trend in infection rates. The most successful new variants have been observed to escape from immune responses generated either by natural infection or vaccination, leading to further infections as well as re-infections. Although boosting of the immune response with the original, historical Spike antigen increases antibody titres³⁶, they are ineffective against future immune evasive variants such as the XBB and BQ.1 lineage³⁶⁻³⁸. In view of these challenges, WHO has recommended the COVID vaccine antigen to match the pre-dominant circulating variant or strain³⁹. However, the problem of “seasonally” immunising with viral Spikes vaccines from viral strains that have (even recently) occurred in the past, will not prevent, and may even drive the continued evolution of vaccine escape variants.

Over the three years of the evolution of SARS-CoV-2 variants, the vaccine composition has been updated twice (BA. 1, BA.4/5) to match the then pre-dominant circulating variants. This is to update the immune landscape of the human population as per the evolving landscape of emergence of SARS-CoV-2 variants. Though the updated vaccine composition has resulted in the increased titre towards the variants in circulation, they have proved to have lower titres against the emerging VOCs. In the current landscape of the ongoing evolution of SARS-CoV-2 variants, it would be more beneficial to have vaccine antigens that can increase breadth of response to both the circulating and potential future variants.

Here, we describe the design of novel spike-based antigens that incorporates critical mutations observed in the evolution of SARS-CoV-2 VOCs. Using sequences of spike protein of Alpha, Beta and Gamma variants, we generated a spike design-T2_32 and compared its immunogenicity to the licensed SARS-CoV-2 Wu-Hu-1 strain. T2_32 generated a superior neutralising response to all the tested VOCs, including the currently circulating variants.

Furthermore, boosting animals immunised with Wu-Hu-1 spike antigen, with the T2_32 vaccine antigen, significantly increased the titres against all the VOCs. This observation validated the utility of T2_32 as a booster antigen to expand the breadth of the immune response encompassing many of the future variants. The immunogenicity of T2_32 validates our rationale that the novel DIOSynVax spike antigens as booster vaccines will provide greater vaccine efficacy against future VOCs compared to the use of historical SARS-CoV-2 variant spike antigens as vaccines. This observation is important in the context of using these new designs for populations that have been primed and boosted with first generation COVID-19 vaccines.

While T2_29 studies were underway, Delta and Omicron lineages arose and became dominant in the human population. The Omicron lineage accrued multiple mutations in both the NTD and RBD regions of the spike protein and was reported to escape previous existing COVID-19 vaccine immunity^{5,34}. As a pre-emptive measure we designed two additional antigens (T2_35 and T2_36). The RBD of T2_35 and the NTD of T2_36 have a maximum identity with the Omicron BA.1 lineage while the NTD of T2_35 and RBD of T2_36 has maximum identity with VOCs prior to emergence of Omicron lineage. We validated the immunogenicity of these candidates by LNP-formulated mRNA immunisation of mice. Both the candidates T2_35 and T2_36 generated a robust neutralising response against all the tested VOCs. Post-immunisation responses with T2_35 were compared to those against BA. 1 spike antigen, and although responses to the Omicron lineage were similar, neutralisation responses were significantly higher against the Delta lineage. In contrast, post-immunisation sera to T2_36 generated lower neutralising titres against Omicron lineage in comparison to BA. 1 spike-antigen, but induced significantly higher neutralising titres in comparison to Wu-Hu-1 spike. The immunogenicity profile of T2_36 against the Omicron lineage in comparison to Wu-Hu-1 spike antigen supports the role of antibodies generated against the NTD/stalk region in broader immune responses against VOCs. From the comparison of the breadth and potency induced in mice across the VOCs tested for, T2_35 was found to have the best immunological profile, followed by T2_36 and BA.1. These studies support human clinical studies to evaluate the immunogenicity of this new class of spike-based antigens as boosters in the background of the complex SARS-CoV-2 immunity in the human population.

In summary, we conclude that antigenically engineered spike antigens have the capacity to induce superior immune breadth than combinations of historical SARS-CoV-2 spike antigens. The broad neutralising responses by these digital immune optimised synthetic spike vaccine candidates support the clinical evaluation of this new class of COVID-19 vaccine antigens.

### REFERENCES

1. Carabelli, A. M. et al. SARS-CoV-2 variant biology: immune escape, transmission and fitness. Nat Rev Microbiol 21, 162-177 (2023).
2. Tan, C. C. S. et al. Transmission of SARS-CoV-2 from humans to animals and potential host adaptation. Nat Commun 13, 2988 (2022).
3. Starr, T. N. et al. Deep Mutational Scanning of SARS-CoV-2 Receptor Binding Domain Reveals Constraints on Folding and ACE2 Binding. Cell 182, 1295-1310.e20 (2020).
4. Qu, P. et al. Neutralization of the SARS-CoV-2 Omicron BA.4/5 and BA.2.12.1 Subvariants. New England Journal of Medicine 386, 2526-2528 (2022).
5. Hu, J. et al. Increased immune escape of the new SARS-CoV-2 variant of concern Omicron. Cell Mol Immunol 19, 293-295 (2022).
6. Lewnard, J. A. et al. Association of SARS-CoV-2 BA.4/BA.5 Omicron lineages with immune escape and clinical outcome. Nat Commun 14, 1407 (2023).
7. Chalkias, S. et al. A Bivalent Omicron-Containing Booster Vaccine against Covid-19. N Engl J Med 387, 1279-1291 (2022).
8. Collier, A. Y. et al. Immunogenicity of BA.5 Bivalent mRNA Vaccine Boosters. N Engl J Med 388, 565-567 (2023).
9. Offit, P. A. Bivalent Covid-19 Vaccines—A Cautionary Tale. New England Journal of Medicine 388, 481-483 (2023).
10. Markov, P. V. et al. The evolution of SARS-CoV-2. Nat Rev Microbiol 21, 361-379 (2023).
11. Krammer, F. The human antibody response to influenza A virus infection and vaccination. Nat Rev Immuno/19, 383-397 (2019).
12. Brister, J. R., Ako-Adjei, D., Bao, Y. & Blinkova, O. NCBI viral genomes resource. Nucleic Acids Res 43, D571-577 (2015).
13. Katoh, K., Misawa, K., Kuma, K. & Miyata, T. MAFFT: a novel method for rapid multiple sequence alignment based on fast Fourier transform. Nucleic Acids Research 30, 3059-3066 (2002).
14. Vita, R. et al. The Immune Epitope Database (IEDB): 2018 update. Nucleic Acids Res 47, D339-D343 (2019).
15. Amanat, F. et al. Introduction of Two Prolines and Removal of the Polybasic Cleavage Site Lead to Higher Efficacy of a Recombinant Spike-Based SARS-CoV-2 Vaccine in the Mouse Model. mBio 12, e02648-20 (2021).
16. Bos, R. et al. Ad26 vector-based COVID-19 vaccine encoding a prefusion-stabilized SARS-CoV-2 Spike immunogen induces potent humoral and cellular immune responses. npj Vaccines 5, 1-11 (2020).
17. Zahradník, J. et al. SARS-CoV-2 variant prediction and antiviral drug design are enabled by RBD in vitro evolution. Nat Microbiol 6, 1188-1198 (2021).
18. Sali, A. & Blundell, T. L. Comparative protein modelling by satisfaction of spatial restraints. J Mol Biol 234, 779-815 (1993).
19. Raab, D., Graf, M., Notka, F., Schodl, T. & Wagner, R. The GeneOptimizer Algorithm: using a sliding window approach to cope with the vast sequence space in multiparameter DNA sequence optimization. Syst Synth Biol 4, 215-225 (2010).
20. Jordan, I. et al. A Deleted D 417 eletion Site in a New Vector Strain and Exceptional Genomic Stability of Plaque-Purified Modified Vaccinia Ankara (MVA). Virol Sin 35, 212-226 (2020).
21. Carnell, G. W. et al. Glycan masking of a non-neutralising epitope enhances neutralising antibodies targeting the RBD of SARS-CoV-2 and its variants. Frontiers in Immunology 14, (2023).
22. Zufferey, R., Nagy, D., Mandel, R. J., Naldini, L. & Trono, D. Multiply attenuated lentiviral vector achieves efficient gene delivery in vivo. Nat Biotechnol 15, 871-875 (1997).
23. Naldini, L. et al. In Vivo Gene Delivery and Stable Transduction of Nondividing Cells by a Lentiviral Vector. http://science.sciencemag.org/.
24. Demaison, C. et al. High-level transduction and gene expression in hematopoietic repopulating cells using a human immunodeficiency [correction of imunodeficiency] virus type 1-based lentiviral vector containing an internal spleen focus forming virus promoter. Human gene therapy 13, 803-813 (2002).
25. Sampson, A. T. et al. Coronavirus Pseudotypes for All Circulating Human Coronaviruses for Quantification of Cross-Neutralizing Antibody Responses. Viruses 13, 1579 (2021).
26. Di Genova, C. et al. Production, Titration, Neutralisation, Storage and Lyophilisation of Severe Acute Respiratory Syndrome Coronavirus 2 (SARS-CoV-2) Lentiviral Pseudotypes. Bio Protoc 11, e4236 (2021).
27. Hoffmann, M. et al. SARS-CoV-2 Cell Entry Depends on ACE2 and TMPRSS2 and Is Blocked by a Clinically Proven Protease Inhibitor. Cell 181, 271-280.e8 (2020).
28. Bertram, S. et al. Influenza and SARS-coronavirus activating proteases TMPRSS2 and HAT are expressed at multiple sites in human respiratory and gastrointestinal tracts. PLOS ONE 7, 1-8 (2012).
29. Carnell, G., Grehan, K., Ferrara, F., Molesti, E. & Temperton, N. J. An Optimised Method for the Production using PEI, Titration and Neutralization of SARS-CoV Spike Luciferase Pseudotypes. Bio-Protocol 7, (2017).
30. Pedregosa, F. et al. Scikit-learn: Machine Learning in Python. Journal of Machine Learning Research 12, 2825-2830 (2011).
31. Hunter, J. D. Matplotlib: A 2D Graphics Environment. Computing in Science Engineering 9, 90-95 (2007).
32. Lu, S. et al. The immunodominant and neutralization linear epitopes for SARS-CoV-2. Cell Rep 34, 108666 (2021).
33. Ujike, M., Huang, C., Shirato, K., Makino, S. & Taguchi, F. The contribution of the cytoplasmic retrieval signal of severe acute respiratory syndrome coronavirus to intracellular accumulation of S proteins and incorporation of S protein into virus-like particles. J Gen Virol 97, 1853-1864 (2016).
34. Fan, Y. et al. SARS-CoV-2 Omicron variant: recent progress and future perspectives. Sig Transduct Target Ther 7, 1-11 (2022).
35. Ju, B. et al. Immune escape by SARS-CoV-2 Omicron variant and structural basis of its effective neutralization by a broad neutralizing human antibody VacW-209. Cell Res 32, 491-494 (2022).
36. Miller, J. et al. Substantial Neutralization Escape by SARS-CoV-2 Omicron Variants BQ.1.1 and XBB.1. New England Journal of Medicine 388, 662-664 (2023).
37. Uraki, R. et al. Antiviral and bivalent vaccine efficacy against an omicron XBB.1.5 isolate. The Lancet Infectious Diseases 23, 402-403 (2023).
38. Yang, J. et al. Low levels of neutralizing antibodies against XBB Omicron subvariants after BA.5 infection. Sig Transduct Target Ther 8, 1-12 (2023).
39. 466 Statement on the antigen composition of COVID-19 vaccines. https://www.who.int/news/item/18-05-2023-statement-on-the-antigen-composition-of-covid-19-vaccines.

### EXAMPLE 8

### Next Generation SuperSpike Family Vaccine Antigens Induce Broad Neutralising Responses Against SARS-CoV-2 Variants of Concern

Next generation SuperSpike designed sequences, CoV_S_T3_1(Deom_v2) (SEQ ID NO:31) and CoV_S_T3_2(Deom_v3) (SEQ ID NO:32) were developed. The next generation sequences are covered by the scaffold sequence SEQ ID NO:30. The amino acid residues of each of CoV_S_T3_1(Deom_v2), and CoV_S_T3_2(Deom_v3), and the first generation designed sequence CoV_S_T2_35(Deom), at the corresponding variable positions of scaffold sequence SEQ ID NO:30, are shown in Example 6.

FIG. 23 shows neutralisation data using mice antisera generated by immunisation with ‘SuperSpike’ vaccine constructs and controls using the mRNA platform against challenge by a panel of SARS-CoV-2 lentiviral pseudoviruses expressing VOC spike protein. Mice were immunised with two doses of 10 μg mRNA in 100 μl vehicle, with a 3-week interval between doses. Mice were bled at 3 weeks after first dose and 6 weeks after first dose. Terminal bleed was 9 weeks after first dose. Next generation ‘SuperSpike’ constructs CoV_S_T3_1 (Deom_v2; SEQ ID NO:31) and CoV_S_T3_2(Deom_v3; SEQ ID NO:32) elicit neutralising antibody responses across the whole diverse panel of omicron PVs and represent a clear improvement over first generation SuperSpike CoV_S_T2_35(Deom; SEQ ID NO:1) across this panel.

1. An isolated polypeptide which comprises an amino acid sequence of SEQ ID NO:1 (CoV_S_T2_35), or an amino acid sequence which has at least 97%, 98%, or 99% amino acid identity over its entire length with the amino acid sequence of SEQ ID NO:1.

2. A polypeptide according to claim 1, which comprises at least one of the amino acid residues of SEQ ID NO: 1, at positions corresponding to the amino acid residue positions of SEQ ID NO: 8, as shown in Table 1 below:
TABLE 1

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 680 | R | G |
| 681 | R | S |
| 683 | R | S |
| 832 | I | V |
| 984 | K | P |
| 985 | V | P |

3. A polypeptide according to claim 1 or 2, which comprises the amino acid residues of SEQ ID NO: 1, at positions corresponding to the amino acid residue positions of SEQ ID NO:8, as shown in Table 3 below:
TABLE 3

| | DeltaC (SEQ | |
|---|---|---|
| DeltaC (SEQ ID | ID NO: 8) | CoV_S_T2_35 |
| NO: 8) residue | reference | (deom) SEQ ID |
| position | residue | NO: 1) |
| 680 | R | G |
| 681 | R | S |
| 683 | R | S |

4. A polypeptide according to any preceding claim, which comprises the amino acid residues of SEQ ID NO:1, at positions corresponding to the amino acid residue positions of SEQ ID NO: 8, as shown in Table 2 below:
TABLE 2

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 832 | I | V |

5. A polypeptide according to any preceding claim, which comprises the following amino acid residues at positions corresponding to residues 984, and 985, of SEQ ID NO:8:
984; P; and
985; P.

6. A polypeptide according to any preceding claim, which comprises the following amino acid residues at positions corresponding to residues 680, 681, 683, 984, and 985, of SEQ ID NO:8:
680; G;
681; S;
683; S;
984; P; and
985; P.

7. A polypeptide according to any preceding claim, which comprises the amino acid residues of SEQ ID NO:1, at positions corresponding to the amino acid residue positions of SEQ ID NO: 8, as shown in Table 1 below:
TABLE 1

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 680 | R | G |
| 681 | R | S |
| 683 | R | S |
| 832 | I | V |
| 984 | K | P |
| 985 | V | P |

8. A polypeptide according to any preceding claim, which does not include residues KFDEDDSEPVLKGVKLHYT at positions corresponding to amino acid residue positions 1253-1271 of SEQ ID NO:8.

9. A polypeptide according to any preceding claim which comprises CoV_S_T2_35(deom) amino acid sequence (SEQ ID NO:1).

10. An isolated polypeptide which comprises a coronavirus S protein with at least one, or all of the amino acid residues, at positions corresponding to the amino acid residue positions of SEQ ID NO:8, as shown in Table 3 below:
TABLE 3

| DeltaC (SEQ | DeltaC (SEQ | |
|---|---|---|
| ID NO:8 ) | ID NO: 8) | CoV_S_T2_35 |
| residue | reference | (deom) SEQ ID |
| position | residue | NO: 1) |
| 680 | R | G |
| 681 | R | S |
| 683 | R | S |

11. A polypeptide according to claim 10, wherein the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:8.

12. A polypeptide according to claim 10 or 11, which comprises the following amino acid residues at positions corresponding to residues 983 and 984, of SEQ ID NO:8:
984; P; and
985; P.

13. A polypeptide according to any of claims 10 to 12, which comprises the amino acid residues of SEQ ID NO:1, at positions corresponding to the amino acid residue positions of SEQ ID NO:8, as shown in Table 2 below:
TABLE 2

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 832 | I | V |

14. A polypeptide according to any of claims 10 to 13, which comprises the amino acid residues of SEQ ID NO:1, at positions corresponding to the amino acid residue positions of SEQ ID NO:8, as shown in Table 1 below:
TABLE 1

| | DeltaC (SEQ | |
|---|---|---|
| DeltaC (SEQ ID | ID NO: 8) | |
| NO: 8) residue | reference | CoV_S_T2_35 (deom) |
| position | residue | SEQ ID NO: 1) |
| 95 | I | T |
| 416 | K | N |
| 438 | N | K |
| 444 | G | S |
| 450 | R | L |
| 475 | S | N |
| 482 | E | A |
| 491 | Q | R |
| 494 | G | S |
| 496 | Q | R |
| 499 | N | Y |
| 503 | Y | H |
| 545 | T | K |
| 675 | Q | H |
| 680 | R | G |
| 681 | R | S |
| 683 | R | S |
| 832 | I | V |
| 984 | K | P |
| 985 | V | P |

15. A polypeptide according to any of claims 10 to 14, which does not include residues KFDEDDSEPVLKGVKLHYT at positions corresponding to amino acid residue positions 1253-1271 of SEQ ID NO:8.

16. An isolated polypeptide, which comprises the amino acid sequence of SEQ ID NO:2 5 (CoV_S_T2_36), or an amino acid which has at least 97%, 98%, or 99% amino acid identity over its entire length with the amino acid sequence of SEQ ID NO:2.

17. A polypeptide according to claim 16, which comprises at least one of the amino acid residues of SEQ ID NO:2, at positions corresponding to the amino acid residue positions of SEQ ID NO:9, as shown in Table 4 below:
TABLE 4

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 679 | R | G |
| 680 | R | S |
| 682 | R | S |
| 983 | K | P |
| 984 | V | P |

18. A polypeptide according to claim 16 or 17, which comprises the amino acid residues of SEQ ID NO:2, at positions corresponding to the amino acid residue positions of SEQ ID NO:9, as shown in Table 6 below:
TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 679 | R | G |
| 680 | R | S |
| 682 | R | S |

19. A polypeptide according to any of claims 16 to 18, which comprises the amino acid residues of SEQ ID NO:2, at positions corresponding to the amino acid residue positions of SEQ ID NO:9, as shown in Table 5 below:
TABLE 5

| Omicron | Omicron | |
|---|---|---|
| BA.1C | BA.1C | |
| (SEQ ID | (SEQ ID | |
| NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |

20. A polypeptide according to any of claims 16 to 19, which comprises the following amino acid residues at positions corresponding to residues 983, and 984, of SEQ ID NO:9:
983; P; and
984; P.

21. A polypeptide according to any of claims 16 to 20, which comprises the following amino acid residues at positions corresponding to residues 679, 680, 682, 983, and 984, of SEQ ID NO: 9:
679; G;
680; S;
682; S;
983; P; and
984; P.

22. A polypeptide according to any of claims 16 to 21, which comprises the amino acid residues of SEQ ID NO:2, at positions corresponding to the amino acid residue positions of SEQ ID NO:9, as shown in Table 4 below:
TABLE 4

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 679 | R | G |
| 680 | R | S |
| 682 | R | S |
| 983 | K | P |
| 984 | V | P |

23. A polypeptide according to any of claims 16 to 22, which does not include residues KFDEDDSEPVLKGVKLHYT at positions corresponding to amino acid residue positions 1252-1270 of SEQ ID NO:9.

24. A polypeptide according to any of claims 16 to 23, which comprises CoV_S_T2_36 (omide) amino acid sequence (SEQ ID NO:2).

25. An isolated polypeptide which comprises a coronavirus S protein with at least one, or all of the amino acid residues, at positions corresponding to the amino acid residue positions of SEQ ID NO:9, as shown in Table 6 below:
TABLE 6

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 679 | R | G |
| 680 | R | S |
| 682 | R | S |

26. A polypeptide according to claim 25, wherein the coronavirus S protein comprises an amino acid sequence which has at least 70%, 75%, 80%, 85%, 90%, 91%, 92%, 93%, 94%, 95%, 96%, 97%, 98%, or 99% amino acid identity over its entire length with the amino acid sequence of SEQ ID NO:9.

27. A polypeptide according to claim 25 or 26, which comprises the following amino acid residues at positions corresponding to residues 983 and 984, of SEQ ID NO:9:
983; P; and
984; P.

28. A polypeptide according to any of claims 25 to 27, which comprises the amino acid residues of SEQ ID NO:2, at positions corresponding to the amino acid residue positions of SEQ ID NO:9, as shown in Table 5 below:
TABLE 5

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |

29. A polypeptide according to any of claims 25 to 28, which comprises the amino acid residues of SEQ ID NO:2, at positions corresponding to the amino acid residue positions of SEQ ID NO:9, as shown in Table 4 below:
TABLE 4

| | Omicron | |
|---|---|---|
| Omicron | BA.1C | |
| BA.1C (SEQ | (SEQ ID | |
| ID NO: 9) | NO: 9) | CoV_S_T2_36 |
| residue | reference | (omide) SEQ ID |
| position | residue | NO: 2) |
| 336 | D | G |
| 368 | L | S |
| 370 | P | S |
| 372 | F | S |
| 437 | K | N |
| 443 | S | G |
| 449 | L | R |
| 474 | N | S |
| 481 | A | E |
| 490 | R | Q |
| 493 | S | G |
| 495 | R | Q |
| 502 | H | Y |
| 544 | K | T |
| 679 | R | G |
| 680 | R | S |
| 682 | R | S |
| 983 | K | P |
| 984 | V | P |

30. A polypeptide according to any of claims 25 to 29, which does not include residues KFDEDDSEPVLKGVKLHYT at positions corresponding to amino acid residue positions 1252-1270 of SEQ ID NO:9.

31. An isolated polypeptide, which comprises an amino acid sequence of SEQ ID NO:3 (Omicron_vaccine), or an amino acid which has at least 99% amino acid identity over its entire length with the amino acid sequence of SEQ ID NO:3.

32. A polypeptide according to claim 31, which comprises at least one of the amino acid residues of SEQ ID NO:3, at positions corresponding to the amino acid residue positions of SEQ ID NO:9, as shown in Table 7 below:
TABLE 7

| | Omicron | |
|---|---|---|
| | BA.1C | |
| | (SEQ ID | |
| Omicron BA.1C | NO: 9) | |
| (SEQ ID NO: 9) | reference | Omicron_vaccine |
| residue position | residue | (SEQ ID NO: 3) |
| 983 | K | P |
| 984 | V | P |

33. A polypeptide according to claim 31 or 32, which comprises the following amino acid residues at positions corresponding to residues 983 and 984, of SEQ ID NO:9:
983; P; and
984; P.

34. A polypeptide according to any of claims 31 to 33, which does not include residues KFDEDDSEPVLKGVKLHYT at positions corresponding to amino acid residue positions 1252-1270 of SEQ ID NO:9.

35. An isolated polypeptide according to any of claims 31 to 34, which comprises an amino acid sequence of SEQ ID NO:3 (Omicron_Vaccine).

36. An isolated polypeptide which comprises an amino acid sequence of SEQ ID NO:30 (CoV_S_T2_35 Scaffold Sequence), wherein X at amino acid residue positions 337, 344, 366, 371, 373, 374, 403, 406, 443, 444, 450, 458, 484, 488, 491, and 494, is any amino acid residue.

37. An isolated polypeptide which comprises an amino acid sequence of SEQ ID NO:33 (RBD portion of CoV_S_T2_35 Scaffold Sequence (SEQ ID NO:30)), wherein X at amino acid residue positions corresponding to amino acid residue positions 337, 344, 366, 371, 373, 374, 403, 406, 443, 444, 450, 458, 484, 488, 491, and 494, of SEQ ID NO:30, is any amino acid residue.

38. An isolated polypeptide according to claim 36 or 37, which comprises amino acid residue G, D, or H at the amino acid residue position corresponding to position 337 of SEQ ID NO:30.

39. An isolated polypeptide according to any of claims 36 to 38, which comprises amino acid residue R or T at the amino acid residue position corresponding to position 344 of SEQ ID NO:30.

40. An isolated polypeptide according to any of claims 36 to 39, which comprises amino acid residue L or I at the amino acid residue position corresponding to position 366 of SEQ ID NO:30.

41. An isolated polypeptide according to any of claims 36 to 40, which comprises amino acid residue S or P at the amino acid residue position corresponding to position 371 of SEQ ID NO:30.

42. An isolated polypeptide according to any of claims 36 to 41, which comprises amino acid residue S or F at the amino acid residue position corresponding to position 373 of SEQ ID NO:30.

43. An isolated polypeptide according to any of claims 36 to 42, which comprises amino acid residue T or A at the amino acid residue position corresponding to position 374 of SEQ ID NO:30.

44. An isolated polypeptide according to any of claims 36 to 43, which comprises amino acid residue D or N at the amino acid residue position corresponding to position 403 of SEQ ID NO:30.

45. An isolated polypeptide according to any of claims 36 to 44, which comprises amino acid residue R or S at the amino acid residue position corresponding to position 406 of SEQ ID NO:30.

46. An isolated polypeptide according to any of claims 36 to 45, which comprises amino acid residue V or P at the amino acid residue position corresponding to position 443 of SEQ ID NO:30.

47. An isolated polypeptide according to any of claims 36 to 46, which comprises amino acid residue S or G at the amino acid residue position corresponding to position 444 of SEQ ID NO:30.

48. An isolated polypeptide according to any of claims 36 to 47, which comprises amino acid residue L or R at the amino acid residue position corresponding to position 450 of SEQ ID NO:30.

49. An isolated polypeptide according to any of claims 36 to 48, which comprises amino acid residue N or K at the amino acid residue position corresponding to position 458 of SEQ ID NO:30.

50. An isolated polypeptide according to any of claims 36 to 49, which comprises amino acid residue F or P at the amino acid residue position corresponding to position 484 of SEQ ID NO:30.

51. An isolated polypeptide according to any of claims 36 to 50, which comprises amino acid residue F or S at the amino acid residue position corresponding to position 488 of SEQ ID NO:30.

52. An isolated polypeptide according to any of claims 36 to 51, which comprises amino acid residue R or Q at the amino acid residue position corresponding to position 491 of SEQ ID NO:30.

53. An isolated polypeptide according to any of claims 36 to 52, which comprises amino acid residue S or G at the amino acid residue position corresponding to position 494 of SEQ ID NO:30.

54. An isolated polypeptide according to any of claims 36 to 53, which comprises an amino acid sequence of SEQ ID NO:31.

55. An isolated polypeptide according to any of claims 36 to 53, which comprises an amino acid sequence of SEQ ID NO:32.

56. An isolated polypeptide according to any of claims 36 to 53, which comprises an amino acid sequence of SEQ ID NO:34.

57. An isolated polypeptide according to any of claims 36 to 53, which comprises an amino acid sequence of SEQ ID NO:35.

58. An isolated polypeptide according to any of claims 36 to 53, which comprises an amino acid sequence of SEQ ID NO:36.

59. An isolated nucleic acid molecule encoding a polypeptide according to any of claims 1 to 58, or the complement thereof.

60. A vector comprising a nucleic acid molecule according to claim 59.

61. A vector according to claim 60, comprising a nucleic acid molecule encoding a polypeptide according to any of claims 1 to 15.

62. A vector according to claim 60, comprising a nucleic acid molecule encoding a polypeptide according to any of claims 16 to 30.

63. A vector according to claim 60, comprising a nucleic acid molecule encoding a polypeptide according to any of claims 31 to 35.

64. A vector according to any of claims to 60 to 63, which further comprises a promoter operably linked to the nucleic acid.

65. A vector according to claim 64, wherein the promoter is for expression of a polypeptide encoded by the nucleic acid in mammalian cells.

66. A vector according to claim 64, wherein the promoter is for expression of a polypeptide encoded by the nucleic acid in yeast or insect cells.

67. A vector according to any of claims 60 to 65, which is a vaccine vector.

68. A vector according to claim 67, which is a viral vaccine vector, a bacterial vaccine vector, an RNA vaccine vector, or a DNA vaccine vector.

69. A vector according to claim 67, which is an mRNA vaccine vector.

70. A vector according to claim 67, which is a DNA vaccine vector.

71. A vector according to claim 67, 68, or 70, which is a Modified Vaccinia virus Ankara (MVA) vector.

72. A vector according to claim 67, 68, or 70, which is a pURVac vector.

73. An isolated cell comprising a vector of any of claims 60 to 72.

74. A fusion protein comprising a polypeptide according to any of claims 1 to 58.

75. A pharmaceutical composition comprising a polypeptide according to any of claims 1 to 58, and a pharmaceutically acceptable carrier, excipient, or diluent.

76. A pharmaceutical composition according to claim 75, comprising a polypeptide according to any of claims 1 to 15.

77. A pharmaceutical composition according to claim 75, comprising a polypeptide according to any of claims 16 to 30.

78. A pharmaceutical composition according to claim 75, comprising a polypeptide according to any of claims 31 to 35.

79. A pharmaceutical composition comprising a nucleic acid molecule according to claim 59, and a pharmaceutically acceptable carrier, excipient, or diluent.

80. A pharmaceutical composition according to claim 79, comprising a nucleic acid molecule encoding a polypeptide according to any of claims 1 to 15.

81. A pharmaceutical composition according to claim 79, comprising a nucleic acid molecule encoding a polypeptide according to any of claims 16 to 30.

82. A pharmaceutical composition according to claim 79, comprising a nucleic acid molecule encoding a polypeptide according to any of claims 31 to 35.

83. A pharmaceutical composition comprising a vector according to any of claims 60 to 72, and a pharmaceutically acceptable carrier, excipient, or diluent.

84. A pharmaceutical composition according to any of claims 75 to 83, which further comprises an adjuvant for enhancing an immune response in a subject to the polypeptide, or to a polypeptide encoded by the nucleic acid, of the composition.

85. A pharmaceutical composition according to any of claims 79 to 82, wherein the nucleic acid molecule is provided by a vector.

86. A pharmaceutical composition according to claim 85, wherein the vector is a vaccine vector.

87. A pharmaceutical composition according to claim 86, wherein the vaccine vector is a viral vaccine vector, a bacterial vaccine vector, an RNA vaccine vector, an mRNA vaccine vector, or a DNA vaccine vector.

88. A pharmaceutical composition according to claim 87, wherein the vaccine vector is a DNA vaccine vector.

89. A pharmaceutical composition according to claim 88, wherein the DNA vaccine vector is a pURVac vector.

90. A pharmaceutical composition according to claim 87, wherein the viral vaccine vector is a Modified Vaccinia virus Ankara (MVA) vector.

91. A pharmaceutical composition according to claim 87, wherein the vaccine vector is an mRNA vaccine vector.

92. A nucleic acid according to claim 59, which comprises one or more modified nucleosides.

93. A vector according to any of claims 60-69, wherein the nucleic acid of the vector comprises one or more modified nucleosides.

94. A pharmaceutical composition according to any of claims 79-82, wherein the nucleic acid of the composition comprises one or more modified nucleosides.

95. A nucleic acid according to claim 92, a vector according to claim 93, or a pharmaceutical composition according to claim 94, wherein the nucleic acid comprises a messenger RNA (mRNA).

96. A nucleic acid according to claim 92 or 95, a vector according to claim 93 or 95, or a pharmaceutical composition according to claim 94 or 95, wherein the one or more modified nucleosides comprise a 1-methylpseudouridine modification.

97. A nucleic acid according to any of claim 92, 95, or 96, a vector according to any of claim 93, 95, or 96, or a pharmaceutical composition according to any of claims 94 to 96, wherein at least 80% of the uridines in the open reading frame have been modified.

98. A pseudotyped virus comprising a polypeptide according to any of claims 1 to 58.

99. A method of inducing an immune response to a coronavirus in a subject, which comprises administering to the subject an effective amount of a polypeptide according to any of claims 1-58, a nucleic acid according to any of claims 59, 92, or 95 to 97, a vector according to any of claim 60-72, 93, or 95-97, or a pharmaceutical composition according to any of claim 75-91, or 94-97.

100. A method of immunising a subject against a coronavirus, which comprises administering to the subject an effective amount of a polypeptide according to any of claims 1-58, a nucleic acid according to any of claims 59, 92, or 95 to 97, a vector according to any of claim 60-72, 93, or 95-97, or a pharmaceutical composition according to any of claim 75-91, or 94-97.

101. A polypeptide according to any of claims 1-58, a nucleic acid according to any of claims 59, 92, or 95 to 97, a vector according to any of claim 60-72, 93, or 95-97, or a pharmaceutical composition according to any of claim 75-91, or 94-97, for use as a medicament.

102. A polypeptide according to any of claims 1-58, a nucleic acid according to any of claims 59, 92, or 95 to 97, a vector according to any of claim 60-72, 93, or 95-97, or a pharmaceutical composition according to any of claim 75-91, or 94-97, for use in the prevention, treatment, or amelioration of a coronavirus infection.

103. Use of a polypeptide according to any of claims 1-58, a nucleic acid according to any of claims 59, 92, or 95 to 97, a vector according to any of claim 60-72, 93, or 95-97, or a pharmaceutical composition according to any of claim 75-91, or 94-97, in the manufacture of a medicament for the prevention, treatment, or amelioration of a coronavirus infection.

104. A method according to claim 99 or 100, a polypeptide, nucleic acid, vector, or pharmaceutical composition, for use according to claim 102, or use according to claim 103, wherein the coronavirus is a beta-coronavirus.

105. A method, or a polypeptide, nucleic acid, vector, or pharmaceutical composition for use, or use according to claim 104, wherein the β-coronavirus is a lineage B or C beta-coronavirus.

106. A method, or a polypeptide, nucleic acid, vector, or pharmaceutical composition for use, or use according to claim 104, wherein the β-coronavirus is a lineage B beta-coronavirus.

107. A method, or a polypeptide, nucleic acid, vector, or pharmaceutical composition for use, or use according to claim 105 or 106, wherein the lineage B β-coronavirus is SARS-CoV or SARS-CoV-2.

108. A method, or a polypeptide, nucleic acid, vector, or pharmaceutical composition for use, or use according to claim 105, wherein the lineage C beta-coronavirus is MERS-COV.

109. A method, or a polypeptide, nucleic acid, vector, or pharmaceutical composition for use, or use according to claim 104, wherein the beta-coronavirus is a variant of concern (VOC).

110. A method, or a polypeptide, nucleic acid, vector, or pharmaceutical composition for use, or use according to claim 104, wherein the beta-coronavirus is a SARS-CoV-2 VOC.

111. A method, or a polypeptide, nucleic acid, vector, or pharmaceutical composition for use, or use according to claim 104, wherein the beta-coronavirus is a SARS-CoV-2 beta, gamma, delta, or omicron VOC.

112. A method of diagnosing whether a subject has a coronavirus infection, which comprises determining whether a polypeptide according to any of claims 1 to 58 is bound by antibodies produced by the subject.

113. A method according to claim 112, wherein the antibodies are in a biological sample obtained from the subject, or in a sample derived from a biological sample obtained from the subject.

114. A method according to claim 113, wherein the biological sample is a serum sample.

115. A method, or a polypeptide, nucleic acid, vector, or pharmaceutical composition for use, or use according to claim 104, wherein the beta-coronavirus is an omicron BA. 1, BA.2, BA.2.75, 2.75.2, BA.2.3.20, BA.4, BA.5, BQ. 1.1, XBB, XBB.1.5, BF.7, XBB.1.19.1, XBC, BQ.1.12, CH.1.1.1, or XBB.1.9.1, virus.


SEQUENCE LISTING (37 sequences)

SEQ ID NO: 1 - protein, 1252 aa, synthetic construct
  REGION 1..1252: note = CoV_S_T2_35 (deom) (designed S protein)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTHTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFVKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 2 - protein, 1251 aa, synthetic construct
  REGION 1..1251: note = CoV_S_T2_36 (omide) (designed S protein)
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 3 - protein, 1251 aa, synthetic construct
  REGION 1..1251: note = Omicron_vaccine
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 4 - protein, 1273 aa, Coronavirus
  REGION 1..1273: note = NC_045512.2_Wuhan_Hu_1 (Wuhan)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1269 aa, synthetic construct
  REGION 1..1269: note = AlphaC
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVANNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 1270 aa, synthetic construct
  REGION 1..1270: note = BetaC
MFVFLVLLPLVSSQCVNLTTRTQLPPSYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 1273 aa, synthetic construct
  REGION 1..1273: note = GammaC
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASFVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1271 aa, synthetic construct
  REGION 1..1271: note = DeltaC
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1270 aa, synthetic construct
  REGION 1..1270: note = Omicron BA.1C
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1270 aa, synthetic construct
  REGION 1..1270: note = Omicron BA.2C
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1270 aa, synthetic construct
  REGION 1..1270: note = Omicron BA.2.12.1C
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1268 aa, synthetic construct
  REGION 1..1268: note = Omicron BA.4C
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1268 aa, synthetic construct; identical to SEQ ID NO: 12
  REGION 1..1268: note = Omicron BA.5C

SEQ ID NO: 14 - protein, 1288 aa, synthetic construct
  REGION 1..1288: note = CoV_T2_1
MFLFLFIIIFAFFLLSAKANERCGIFTSKPQPKLAQVSSSRRGVYYPDDIFRSDVLHLTQDYFLPFDSNVTRYFSLNANGPDRIVYFDNPIIPFKDGVYFAATEKSNVIRGWIFGSTLDNTSQSVIIVNNSTNVIIRVCNFDLCNDPFFTVSRPTDKHIKTWSIREFAVYQSAFNCTFEYVSKSFLLDVAEKPGNFKHLREFVFKNVDGFLNVYSTYKPINVVSGLPTGFSVLKPILKLPLGINITSFRVLLTMFRGDPTPGHTTANWLTAAAAYYVGYLKPTTFMLKYNENGTITDAVDCSQNPLAELKCTLKNFNVDKGIYQTSNFRVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQLVKNQCVNFNFNGLKGTGVLTASSKRFQSFQQFGRDASDFTDSVRDPQTLEILDISPCSFGGVSVITPGTNTSSEVAVLYQDVNCTDVPTAIHADQLTPAWRVYSTGVNVFQTQAGCLIGAEHVNASYECDIPIGAGICASYHTASNSPRILRSTGQKSIVAYTMSLGAENSIAYANNSIAIPTNFSISVTTEVMPVSMAKTSVDCTMYICGDSLECSNLLLQYGSFCTQLNRALTGIAIEQDKNTQEVFAQVKQMYKTPAIKDFGGFNFSQILPDPSKPTKRSFIEDLLFNKVTLADAGFMKQYGECLGDISARDLICAQKFNGLTVLPPLLTDEMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVSNGTSWFITQRNFYSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMATILLCCMTSCCSCLKGACSCGSCCKFDEDDSEPVLKGVKLHYT

SEQ ID NO: 15 - protein, 214 aa, synthetic construct
  REGION 1..214: note = CoV_T2_8 (M7 RBD)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHANATVCGPKKSTN

SEQ ID NO: 16 - protein, 214 aa, synthetic construct
  REGION 1..214: note = CoV_T2_9 (M8 RBD)
RVQPTESIVRFPQITNLCPFGEVFQATRFASVYAWNRKRISNCVADYSVLYNSTSFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTN

SEQ ID NO: 17 - protein, 219 aa, synthetic construct
  REGION 1..219: note = CoV_T2_10 (M9 RBD)
RVSPTQEVVRFPNITNLCPFDKVFNATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNANATVCGPKLSTQ

SEQ ID NO: 18 - protein, 219 aa, synthetic construct
  REGION 1..219: note = CoV_T2_11 (M10 RBD)
RVSPTQEVVRFPQITNLCPFDKVFQATRFPSVYAWERTKISDCVADYTVLYNSTSFSTFKCYGVSPSKLIDLCFTSVYADTFLIRCSEVRQVAPGQTGVIADYNYKLPDDFTGCVIAWNTAKQDTGSSGNYNYYYRSHRKTKLKPFERDLSSDECSPDGKPCTPPAFNGVRGFNCYFTLSTYDFNPNVPVEYQATRVVVLSFELLNAPATVCGPKLSTQ

SEQ ID NO: 19 - protein, 214 aa, synthetic construct
  REGION 1..214: note = CoV_T2_13 (designed S protein RBD sequence)
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 20 - protein, 214 aa, synthetic construct
  REGION 1..214: note = CoV_T2_14 (designed S protein RBD sequence)
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 21 - protein, 214 aa, synthetic construct
  REGION 1..214: note = CoV_T2_15 (designed S protein RBD sequence)
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTD

SEQ ID NO: 22 - protein, 214 aa, synthetic construct
  REGION 1..214: note = CoV_T2_16 (designed S protein RBD sequence)
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGKIADYNYKLPDDFTGCVIAWNTNNLDSTTGGNYNYLYRLFRKSNLKPFERDISSDIYQAGSTPCSGVEGFNCYFPLQSYGFQPTNGVGYQPYRVVVLSFELLNAPATVCGPKLSTD

SEQ ID NO: 23 - protein, 214 aa, synthetic construct
  REGION 1..214: note = CoV_T2_17 (designed S protein RBD sequence)
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 24 - protein, 214 aa, synthetic construct
  REGION 1..214: note = CoV_T2_18 (designed S protein RBD sequence)
RVAPTKEVVRFPNITNLCPFGEVFNATRFPSVYAWERKRISNCVADYSVLYNSTFFSTFKCYGVSPTKLNDLCFSNVYADSFVIRGDEVRQIAPGQTGVIADYNYKLPDDFMGCVIAWNTNNLDSTTGGNYNYLYRSLRKSKLKPFERDISSDIYSPGGKPCSGVEGFNCYYPLRSYGFFPTNGTGYQPYRVVVLSFELLNAPATVCGPKLSTD

SEQ ID NO: 25 - protein, 284 aa, synthetic construct
  REGION 1..284: note = CoV_T2_19 (designed S protein RBD sequence)
RVAPTKEVVRFPNITNLCPFGEVFNATRFPSVYAWERKRISNCVADYSVLYNSTSFSTFKCYGVSPTKLNDLCFTNVYADSFVIRGDEVRQIAPGQTGVIADYNYKLPDDFTGCVIAWNTNNLDSTTGGNYNYLYRSLRKSKLKPFERDISSDIYSPGGKPCSGVEGFNCYYPLRSYGFFPTNGVGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 26 - protein, 284 aa, synthetic construct
  REGION 1..284: note = CoV_T2_20 (designed S protein RBD sequence)
RVAPTKEVVRFPNITNLCPFGEVFNATKFPSVYAWERKKISNCVADYSVLYNSTSFSTFKCYGVSPTKLNDLCFTNVYADSFVIRGDEVRQIAPGQTGVIADYNYKLPDDFTGCVIAWNTNNIDSTTGGNYNYLYRSLRKSKLKPFERDISSDIYSPGGKPCSGVEGFNCYYPLRSYGFFPTNGTGYQPYRVVVLSFELLNAPATVCGPKLSTDGGGGSGGGGSGGGGSGGGGSKSSIASFFFIIGLIIGLFLVLRVGIHLCIKLKHTKKRQIYTDIEMNRLGK

SEQ ID NO: 27 - protein, 1270 aa, synthetic construct
  REGION 1..1270: note = CoV_T2_29 (T2_29)
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - protein, 1270 aa, synthetic construct
  REGION 1..1270: note = CoV_T2_31 (T2_29+Q498R)
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFRPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 29 - protein, 1251 aa, synthetic construct
  REGION 1..1251: note = CoV_T2_32 (T2_29+Q498R+dER)
MFVFLVLLPLVSSQCVNFTNRTQLPSAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFRPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 30 - protein, 1252 aa, synthetic construct
  REGION 1..1252: note = CoV_S_T2_35 Scaffold Sequence
  VARIANT 337..494: note = X can be any amino acid
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFXEVFNATXFASVYAWNRKRISNCVADYSVXYNSAXFXXFKCYGVSPTKLNDLCFTNVYADSFVIRGXEVXQIAPGQTGNIADYNYKLPDDFTGCVIAWNSNKLDSKXXGNYNYXYRLFRKSXLKPFERDISTEIYQAGNKPCNGVAGXNCYXPLXSYXFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTHTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFVKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 31 - protein, 1252 aa, synthetic construct
  REGION 1..1252: note = COV_S_T3_1(Deom_v2)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSKLKPFERDISTEIYQAGNKPCNGVAGPNCYFPLQSYGFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTHTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFVKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 32 - protein, 1252 aa, synthetic construct
  REGION 1..1252: note = COV_S_T3_2(Deom_v3)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTHTNSRGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFVKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCC

SEQ ID NO: 33 - protein, 214 aa, synthetic construct
  REGION 1..214: note = RBD portion of CoV_S_T2_35 Scaffold Sequence (SEQ ID NO:30)
  VARIANT 21..178: note = X can be any amino acid
RVQPTESIVRFPNITNLCPFXEVFNATXFASVYAWNRKRISNCVADYSVXYNSAXFXXFKCYGVSPTKLNDLCFTNVYADSFVIRGXEVXQIAPGQTGNIADYNYKLPDDFTGCVIAWNSNKLDSKXXGNYNYXYRLFRKSXLKPFERDISTEIYQAGNKPCNGVAGXNCYXPLXSYXFRPTYGVGHQPYRVVVLSFELLHAPATVCGPKKSTN

SEQ ID NO: 34 - protein, 214 aa, synthetic construct
  REGION 1..214: note = RBD portion of CoV_S_T2_35(deom) (SEQ ID NO:1)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTN

SEQ ID NO: 35 - protein, 214 aa, synthetic construct
  REGION 1..214: note = RBD portion of COV_S_T3_1(Deom_v2) (SEQ ID NO:31)
RVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSKLKPFERDISTEIYQAGNKPCNGVAGPNCYFPLQSYGFRPTYGVGHQPYRVVVLSFELLHAPATVCGPKKSTN

SEQ ID NO: 36 - protein, 214 aa, synthetic construct
  REGION 1..214: note = RBD portion of COV_S_T3_2(Deom_v3) (SEQ ID NO:32)
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTN

SEQ ID NO: 37 - protein, 19 aa, synthetic construct
  REGION 1..19: note = amino acid residue positions 1252-1270 of SEQ ID NO:9
KFDEDDSEPVLKGVKLHYT